(19) United States
(12) Patent Application Publication
(10) Pub. No.: US 2024/0252477 A1
(43) Pub. Date: Aug. 1, 2024

(54) NLRP3 INFLAMMASOME-INHIBITING COMPOUNDS AND THE USE THEREOF

(71) Applicants: UNIVERSITA' DEGLI STUDI DI TORINO (60%), Torino (IT); UNIVERSITA' DI PISA (40%), Pisa (IT)

(72) Inventors: Corrado Blandizzi, Pisa (PI) (IT); Carolina Pellegrini, Pisa (PI) (IT); Matteo Fornai, Pisa (PI) (IT); Luca Antonioli, Pisa (PI) (IT); Rocchina Colucci, Padova (PD) (IT); Massimo Bertinaria, Torino (TO) (IT); Elisabetta Marini, Torino (TO) (IT); Valentina Boscaro, Torino (TO) (IT); Simone Gastaldi, Torino (TO) (IT); Mattia Cocco, Torino (TO) (IT)

(73) Assignees: UNIVERSITA' DEGLI STUDI DI TORINO (60%), Torino (IT); UNIVERSITA' DI PISA (40%), Pisa (IT)

(21) Appl. No.: 18/558,810

(22) PCT Filed: May 3, 2022
(86) PCT No.: PCT/IB2022/054072

§ 371 Date: Nov. 3, 2023

(30) Foreign Application Priority Data
May 3, 2021 (IT) 102021000011237

(51) Int. Cl. A61K 31/445; A61K 31/192; A61K 31/222; A61K 31/265; A61K 31/40

(52) U.S. Cl. CPC A61K 31/445; A61K 31/192; A61K 31/222; A61K 31/265; A61K 31/40

(57) ABSTRACT

The invention relates to compounds of general formula (I), having inhibitory activity against the NLRP3 inflammasome. Said compounds are useful in the prevention and/or treatment of diseases and/or disorders mediated by the NLRP3 inflammasome.

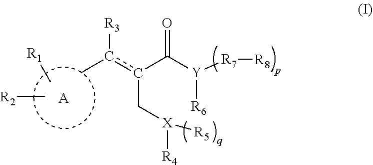

### TECHNICAL FIELD OF INVENTION

The invention relates to NLRP3 inflammasome-inhibiting compounds which are particularly useful in the prevention and/or treatment of diseases and/or disorders mediated by the NLRP3 inflammasome.

### PRIOR ART

The discovery that the immune system and the inflammatory processes correlated with its chronic activation underlie a huge number of disorders which account for the highest global morbidity and mortality figures was one of the major medical discoveries of the last twenty years [Netea et al. 2017, Slavich 2015, Bennett, et al. 2018].

Chronic inflammatory disorders are now recognised as the main cause of death worldwide, over 50% of deaths being attributable to disorders accompanied by various inflammatory states (acute or chronic), such as acute myocardial ischaemia, stroke, type 2 diabetes, chronic kidney failure, non-alcoholic steatohepatitis (NASH), numerous autoimmune and neurodegenerative diseases, and some forms of cancer [Straub and Schradin 2016, Furman et al. 2019, GBD 2017 Causes of Death Collaborators 2018].

Inflammation is a process activated by the host's immune system in response to stimuli recognised as harmful, such as the presence of irritants, pathogens and the products thereof, and in response to excessive cell death. Inflammasomes are intracellular complexes which act as “sensors” of the innate immune system and perform the role of main promoters of the inflammatory response by triggering a cascade of events that cause secretion of pro-inflammatory cytokines interleukin (IL)-1beta (interleukin-1β or IL-1β) and IL-18, and cell death by pyroptosis. The NLRP3 inflammasome is the most widely studied of the inflammasomes, because it is involved in numerous pathological processes.

The NLRP3 inflammasome is a multiprotein complex consisting of protein NLRP3 (NOD-, LRR- and pyrin domain-containing protein 3) which assembles in the cytosol with ASC protein (apoptosis-associated speck-like protein containing a CARD) and procaspase-1, forming an oligomeric aggregate called an inflammasome, which is capable of causing autoproteolysis of procaspase-1, generating the active form of the protease called caspase-1. The latter then cleaves pro-IL-1beta and pro-IL-18 generating IL-1beta and IL-18, which cause a powerful inflammatory response. Moreover, the caspase-1 activated by NLRP3 cleaves the protein gasdermin-D, forming gasdermin-N, which latter forms pores in the cell membrane that lead to cell death by the process known as pyroptosis [Groslambert and Py 2018, He et al. 2016] and to the release of proinflammatory material into the extracellular space.

Abnormal activation and hyperactivation of NLRP3 are undoubtedly involved in numerous acute and chronic inflammatory disorders [Mangan et al. 2018].

The physiological role of NLRP3 is not yet fully understood, and no mutations inactivating gene nlrp3 have been described to date. Conversely, mutations activating in gene nlrp3 (NALP3 or CIAS-1) generate an NLRP3 protein which is continuously activated. Said mutations are the etiological factor of a set of autoinflammatory disorders known as cryopyrinopathies (CAPS) [Booshehri and Hoffman 2019, Mortimer et al. 2016].

Data obtained from studies of animal models and supported by studies on patients demonstrate that activation of NLRP3 leads to a chronic inflammatory state that can cause, accompany and promote numerous pathological processes which have a major impact on public health [Fusco et al. 2020]. The main disorders correlated with increased, uncontrolled activation of NLRP3 are: (i) metabolic and cardiovascular disorders such as atherosclerosis [Jing and Fu 2019], type 2 diabetes [Lee et al. 2013], inflammation induced by obesity and insulin resistance [Vandanmagsar et al. 2011, Yin et al. 2014], myocardial ischaemia [Wang et al. 2014, Toldo and Abbate 2018], stroke [Ren et al. 2018.]; (ii) chronic inflammatory disorders that seriously affect various organs and tissues in a progressively degenerative manner. Among said disorders, uncontrolled activation of NLRP3 has been found in inflammatory bowel diseases such as Crohn's disease and ulcerating colitis [Liu et al. 2017, Zhen and Zhang 2019], various forms of arthritis, including rheumatoid arthritis [Vande Walle et al. 2014, Guo et al. 2018], systemic lupus erythematosus, Sjögren's syndrome, ankylosing spondylitis, systemic sclerosis [Li et al 2020], gout [Martinon et al. 2006, Szekanecz et al. 2019], non-alcoholic steatohepatitis (NASH) and hepatic fibrosis [Boaru et al. 2012, Mridha et al 2017, Wu et al. 2017]; (iii) inflammatory disorders of the airways [Primiano et al. 2016, Theofani et al. 2019], including the severe inflammatory pulmonary complications correlated with Sars-CoV-2 infection (COVID-19) [Freeman and Swartz 2020]; (iv) neurodegenerative disorders such as Alzheimer's disease, Parkinson's disease, amyotrophic lateral sclerosis, head injury [Heneka et al. 2018], and multiple sclerosis [Malhotra et al. 2020]; (v) other disorders, such as sepsis [Cornelius et al. 2020], sterile corneal inflammation [Shimizu et al. 2019] and some myelodysplastic syndromes [Basiorka et al. 2016, Ratajczak et al. 2020], have been correlated with hyperactivation of the inflammasome.

These clinical and experimental observations demonstrate that the NLRP3 inflammasome is an extremely interesting target for the discovery of new medicaments for the treatment of disorders for which an optimum treatment is not yet available [Chauhan et al. 2020].

Biopharmaceuticals that block IL-1beta (anakinra, canakinumab and rilonacept) are already present on the market and used to treat various inflammatory disorders. Anakinra is approved for the treatment of cryopyrinopathies, rheumatoid arthritis, colchicine-resistant Familial Mediterranean Fever (FMF) and Schnitzler syndrome. Canakinumab is approved for the treatment of cryopyrinopathies, Familial Mediterranean Fever (FMF), tumour necrosis factor receptor-associated periodic syndrome (TRAPS), hyperimmunoglobulinaemia D syndrome/mevalonate kinase deficiency (HIDS/MKD), idiopathic juvenile arthritis, gouty arthritis and adult Still's disease. The CANTOS clinical trial (NCT01327846) also demonstrated significant activity in reducing secondary cardiovascular events and strokes and preventing mortality due to cardiovascular events (−31%) in patients at cardiovascular risk. The CANTOS study also demonstrated a 77% reduction in lung cancer deaths after treatment with canakinumab. Rilonacept is approved by the FDA for treatment of cryopyrinopathies (Familial Mediterranean Fever (FMF) and Muckle-Wells syndrome (MWS). Although it is effective, the clinical use of IL-1beta blockers involves some recognised, problematic limitations, which have not yet been resolved. The main limitations of treatment based on the use of IL-1beta blockers are as follows:
1) total blocking of the effects of IL-1beta, obtained with the blockers on the market, makes patients more liable to infection. Clinical treatment with anakinra and canakinumab has demonstrated an increased risk of infections of the upper airways and urinary tract caused by Escherichia coli and Streptococci. Other significant side effects associated with IL-1beta inhibitor treatment are neutropenia and thrombocytopenia.
2) In view of the limited stability and non-ideal pharmacokinetics of biological IL-1beta blockers, their clinical use requires said medicaments to be administered by injection, usually in hospital. This considerably limits patient compliance. The frequency of administration can range from daily (anakinra) to weekly (rilonacept) or every eight weeks (canakinumab), and the treatment is lifelong.
3) IL-1beta blocker treatment is expensive.
4) IL-1beta blockers do not block the inflammatory response mediated by IL-18 or cell death by pyroptosis, which amplifies and supports the inflammatory response.

The development of synthetic molecules able to inhibit activation of the inflammatory signalling pathway by directly inhibiting the NLRP3 inflammasome would overcome the limitations of the treatments currently available, and increase the number of disorders treatable with said medicaments. In particular, selective inhibition of the NLRP3 inflammasome would block the signalling pathway leading to release of IL-1beta upstream, thus reducing the secretion of said inflammatory cytokine. The generalised inhibition of IL-1beta which is obtained with the blockers currently on the market gives rise to immunosuppression and increased risk of infection. Inhibiting the NLRP3 inflammasome would enable other inflammasomes (AIM2, NLRC4, NLRP1) present in the cells of the innate immune system to produce IL-1beta. This would minimise the risks of severe immunosuppression and infection.

Small molecules able to inhibit NLRP3 could have physicochemical properties allowing their oral administration, thus greatly simplifying the therapeutic dosage regimen and increasing patient compliance.

Moreover, the cost of a treatment with synthetic NLRP3 inhibitors would be lower than the cost of treatment with biological IL-1beta blockers, leading to a considerable saving for the national health service.

Unlike IL-1beta blockers, NLRP3 inhibitors can also block the inflammatory responses due to IL-18 secretion and cell death by pyroptosis, which amplifies and supports the inflammatory process, releasing “cell debris” (Danger-Associated Molecular Patterns; DAMP) and other pro-inflammatory mediators by means of cell lysis. As a result of said molecular mechanism, the number of disorders treatable with an NLRP3 inhibitor is undoubtedly higher than that of the disorders treatable with a simple IL-1beta inhibitor. This benefit of selective NLRP3 inhibitors has been demonstrated in a study of NLRP3 knock-out mice, which demonstrated that there was no greater risk of infection than for wild-type mice. Moreover, the mice lacking NLRP3 did not exhibit any metabolic defects, thus demonstrating the safety of the therapeutic strategy [Youm et al. 2013].

Although some molecules able to inhibit the activation or assembly of the NLRP3 inflammasome more or less selectively have been discovered, there are still no NLRP3 inflammasome inhibitors on the market [Zahid et al. 2019, Bertinaria et al. 2019]. The molecule whose development is at the most advanced stage is dapansutrile (OLT-1177; Olatec Therapeutics LLC, New York, NY, USA). Said NLRP3 inhibitor is currently in phase 2a of development, and is undergoing a clinical trial for the treatment of gouty arthritis (EUDRACT number: 2016-000943-14). The results published to date are favourable, indicating good safety, tolerability and activity [Kluck et al. 2020]. The company Inflazome (Inflazome UK Ltd, Cambridge, UK) is developing inzomelid and somalix, NLRP3 inhibitors which are both in phase 1 of clinical development (WO 2016/131098, U.S. Pat. No. 10,538,487 and EP 3.259,253). Said molecules derive from modulation of molecule MCC950, the reference NLRP3 inhibitor currently used in pharmacological studies.

There is consequently still a need to identify compounds able to inhibit the NLRP3 inflammasome which are useful in the prevention and/or treatment of diseases and/or disorders mediated by the NLRP3 inflammasome.

### LIST AND BRIEF DESCRIPTION OF FIGURES

FIG. 1 shows the effect of INF176 treatment (1-20 μM) on (A) pyroptosis of human macrophages stimulated with LPS/ATP; (B) release of IL-1beta from human macrophages stimulated with LPS/ATP. *p<0.05 vs ATP. Statistics: Student's t-test.

FIG. 2 shows the effect of INF176 treatment at the dose of 25 and 50 mg/kg on (A) variation in body weight and (B) spleen weight. *p<0.05 vs CTR (control) and tp<0.05 vs DSS. Statistics: ANOVA analysis followed by Tukey's post hoc test.

FIG. 3 shows the effect of INF176 treatment at the dose of 25 and 50 mg/kg on: (A) colon length, (B) disease activity index (DAI), (C) myeloperoxidase (MPO) levels in colon, and (D) interleukin-1beta (IL-1β) levels in colon. *p<0.05 vs CTR (control) and tp<0.05 vs DSS. Statistics: ANOVA analysis of compounds, followed by Tukey's post hoc test.

FIG. 4 shows the effect of INF176 treatment at the dose of 50 mg/kg on: (A) escape latency, (B) number of crossings in quadrant, (C) number of entries into quadrant, (D) p-tau protein expression in brain tissues, (E) β amyloid 1-42 (Aβ1-42) protein levels in brain tissues. ^{a}p<0.05 vs SAMR1 (control), *p<0.05 vs SAMPS. Statistics: ANOVA analysis followed by Tukey's post hoc test.

FIG. 5 shows the effect of INF176 treatment at the dose of 50 mg/kg on: (A) number of pellets expelled in 1 hour, (B) colon contractions elicited by electrical stimuli, (B) cholinergic colon contractions elicited by electrical stimuli, (B) tachykininergic colon contractions elicited by electrical stimuli. ^{a}p<0.05 vs SAMR1 (control), *p<0.05 vs SAMP8. Statistics: ANOVA analysis followed by Tukey's post hoc test.

FIG. 6 shows the effect of INF176 treatment at the dose of 50 mg/kg on interleukin-1beta (IL-1β) levels in the colon. Statistics: ANOVA analysis followed by Tukey's post hoc test.

FIG. 7 shows the chromatogram of the HPLC analysis conducted to determine the stability of INF177 in PBS in the presence of glutathione 10×.

FIG. 8 shows the chromatogram of the HPLC analysis conducted to determine the stability of INF177 in PBS in the presence of cysteamine 10×.

### SUMMARY OF THE INVENTION

The object of the present invention is compounds of general formula (I), and the corresponding sub-formulae:

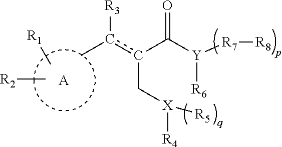

wherein R₁, R₂, R₃, R₄, R₅, R₆, R₇, R₈, q, p, X and Y are as defined below,

and their enantiomers, diastereomers, rotamers or mixtures thereof;

and the pharmaceutically acceptable salts or solvates thereof.

The invention also relates to compositions containing at least one compound of general formula (I), (Ia), (Ib) or (Ic) as defined below, and at least one pharmaceutically acceptable excipient or carrier.

Further objects of the invention are compounds of general formula (I) for use as a medicament, in particular to inhibit the NLRP3 inflammasome.

According to a further aspect, the invention relates to compounds of general formula (I) for use in the prevention and/or treatment of inflammatory, autoimmune, neurodegenerative, cardiovascular, metabolic and neoplastic diseases and/or disorders.

### DETAILED DESCRIPTION OF THE INVENTION

The object of the present invention is compounds of general formula (I):

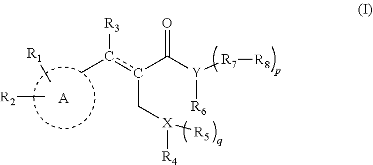

wherein:

A is a C₃-C₁₀-cycloalkyl, preferably monocyclic or bicyclic C₅-C₁₀-cycloalkyl; 5-to 10-membered, saturated or partly saturated, monocyclic or bicyclic heterocycle; monocyclic or bicyclic C₆-C₁₀-aryl; 5- to 10-membered monocyclic or bicyclic heteroaryl; A is preferably a 5 or 6-membered, saturated or partly saturated, monocyclic heterocycle, or a 9 or 10-membered, saturated or partly saturated, bicyclic heterocycle; or a monocyclic C₅-C₆-aryl, or a bicyclic C₉-C₁₀-aryl; or a 5 or 6-membered monocyclic heteroaryl or a 9 or 10-membered bicyclic heteroaryl; wherein the heteroatom is preferably N or O; more preferably A is phenyl, naphthyl, furanyl or indolyl, and most preferably A is phenyl;

R₁ and R₂, which are the same or different, can occupy any position on A, and can be hydrogen; halogen such as F, Cl, Br or I; linear or branched, substituted or unsubstituted, saturated or unsaturated C₁-C₄-alkyl; linear or branched, substituted or unsubstituted, saturated or unsaturated C₁-C₄-alkoxy; a nitro group; nitrile; a substituted or unsubstituted amido group; a substituted or unsubstituted amino group; a substituted or unsubstituted ester group; a trifluoromethyl group; R₁ and R₂ are preferably hydrogen, halogen such as F, Cl, Br or I, linear or branched C₁-C₄-alkyl, linear or branched C₁-C₄-alkoxy, a nitro group; R₁ and R₂ are more preferably hydrogen, chloro, bromo, methyl, methoxy or a nitro group; most preferably R₁ is hydrogen and R₂ is chloro; R₁ or R₂ is preferably in the 2 position when A is phenyl;

[symbol] can be a single bond or a double bond;

R₃ can be —H, —OH, —OR₉ or —O(CO)R₉, wherein R₉ can be hydrogen, a linear or branched C₁-C₄-alkyl, substituted or unsubstituted, saturated or unsaturated; R₃ is preferably hydrogen or —OH; R₃ is more preferably hydrogen;

in the

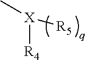

group X can be N, O, S, S(O) or SO₂;

R₄ can be a linear or branched, substituted or unsubstituted, saturated or unsaturated C₁₋₄ alkyl group; monocyclic or bicyclic C₃-C₁₀-cycloalkyl, substituted or unsubstituted, preferably a C₃-C₆-cycloalkyl; monocyclic or bicyclic C₆-C₁₄-aryl, substituted or unsubstituted, preferably a C₆-C₁₀-aryl, more preferably a C₅-C₆-aryl; 5- to 10-membered heterocycle, saturated or partly saturated, monocyclic or bicyclic, substituted or unsubstituted, preferably a C₅-C₆-heterocycle; monocyclic or polycyclic 5- to 14-membered heteroaryl, preferably monocyclic or bicyclic, substituted or unsubstituted, preferably a C₅-C₆-heteroaryl; R₄ is preferably monocyclic or bicyclic C₆-C₁₀-aryl, substituted or unsubstituted, or C₃-C₆-cycloalkyl, substituted or unsubstituted; more preferably, R₄ is cyclohexyl or phenyl;

q can be 0 (zero) or 1; when q is equal to 1, X is N and R is hydrogen; a linear or branched, substituted or unsubstituted, saturated or unsaturated C₁-C₄-alkyl group; monocyclic or bicyclic C₃-C₁₀-cycloalkyl;

alternatively, the

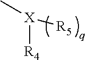

group can be an amino-acid residue wherein:
X is an N, S or O atom of the side chain of an amino acid, preferably natural, selected from serine; tyrosine; threonine; lysine; cysteine; q is zero (R₅ is therefore not present) and R₄ is the remainder of the amino acid which can be protected or unprotected on the NH₂ and/or COOH terminal groups; in a preferred aspect the terminal NH₂ group is acetylated; in a preferred aspect, the amino-acid residue is N-acetyleysteine or N-Boc cysteine methyl ester; or
X is the N atom of the terminal amino group bonded to the stereogenic carbon atom in alpha of a preferably natural, protected or unprotected, amino acid, selected from alanine, arginine, asparagine, aspartic acid, cysteine, glycine, glutamic acid, glutamine, histidine, isoleucine, leucine, lysine, methionine, phenylalanine, proline, serine, threonine, tryptophan, tyrosine and valine; q is equal to 1, R₅ is hydrogen; and R₄ represents the remainder of the amino-acid structure, protected or unprotected, for example acetylated on the N atom of the side chain or esterified with a linear or branched C₁-C₄-alkyl group, preferably methyl, on the terminal carboxyl group;

alternatively when, in the

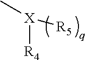

group, X is N, R₄ and R₅ can be joined to form a saturated, partly saturated or unsaturated, monocyclic or bicyclic C₃-C₁₀-heterocyclic ring with the N atom; R₄ and R₅ preferably form a monocyclic C₃-C₆-heterocyclic ring with the N atom; more preferably, R₄ and R₅ form a piperidine or pyrrolidine ring with the N atom; most preferably, R₄ and R₅ form a pyrrolidine ring with the N atom;

Y can be selected from O, N and S; is preferably O or N; and is more preferably N;

when Y is an oxygen or sulfur atom, in the —(R₇-R₈)p group p is equal to zero and R₆ can be hydrogen, a linear or branched, substituted or unsubstituted, saturated or unsaturated C₁-C₈-alkyl group; a monocyclic or bicyclic C₃-C₁₀-cycloalkyl; a substituted or unsubstituted arylalkyl; a 6- to 14-membered monocyclic or bicyclic heteroaryl; R₆ is preferably hydrogen or a linear or branched, substituted or unsubstituted, saturated or unsaturated C₁-C₄-alkyl group; R₆ is more preferably a linear or branched, saturated, unsubstituted C₁-C₄-alkyl group; R₆ is most preferably ethyl;

when Y is a nitrogen atom, p is equal to 1, R₆ and R₇, which are the same or different, are selected from hydrogen, a linear or branched, saturated or unsaturated, substituted or unsubstituted C₁-C₄-alkyl group; a substituted or unsubstituted aryl, arylalkyl or heteroaryl group; preferably, it can be a substituted phenylalkyl group; more preferably —(CH₂)₂-phenyl-SO₂NH₂; most preferably R₆ is hydrogen and R₇ is —(CH₂)₂-phenyl-SO₂NH₂;

R₈ can be selected from H, COOH, COOR₉, C(O)R₉, CN, CONH(R₉), S(O)NHR₉ and S(O)₂NHR₉, wherein R₉ is as defined above;

alternatively, R₆ and R₇ can be joined to form a 3- to 8-membered heterocyclic ring;

R₈ is as defined above;

and their enantiomers, diastereomers, rotamers or mixtures thereof;

and the pharmaceutically acceptable salts or solvates thereof;

for use as a medicament, in particular for use in the prevention and/or treatment of NLRP3 inflammasome-mediated diseases and/or disorders.

According to one embodiment of the invention, [symbol] is a single bond and A, R₁, R₂, R₃, R₄, R₅, R₆, R₇, R₈, q, p, X and Y are as defined above.

According to a further embodiment, when [symbol] is a double bond, X is N or O, A is phenyl, R₁ is in the 2 position and is preferably a halogen, more preferably chloro, R₂, R₃, R₄, R₅, R₆, R₇, R₈, q, p and Y are as defined above.

According to a preferred aspect, the compounds for use according to the invention have general formula (Ia), when R₆ and R₇ form a ring:

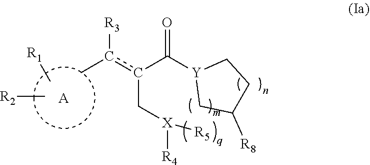

wherein

A, R₁, R₂, R₃, R₄, R₅, R₈, q and X are as defined above;

n and m, which are the same or different, can be 0 (zero) or an integer between 1 and 3; when n and m are equal to zero, a three-membered cycle is generated between C—R₈, Y, and the remaining —CH₂— group; when n and m, which are different from one another, are 0 (zero) or 1, a 4-membered ring is formed; or n and m, which are the same or different, can be 1, 2 or 3 forming 5- to 9-, preferably 5- to 8-membered rings; according to a preferred aspect, m is 2 and n is 1; according to a more preferred aspect, n is 2 and m can be 1 or 2; most preferably, n is 2 and m is 1;

preferably, when Y is N, R₆ and R₇ can be joined to form a 3- to 6-membered monocyclic substituted heterocyclic ring with the N atom; more preferably, R₆ and R₇ form a substituted piperidine or pyrrolidine ring with the N atom; most preferably, R₆ and R₇ form, with the N atom, a piperidine ring substituted in the 3 or 4 position.

According to a further preferred aspect the compounds for use according to the invention have general formula (Ib):

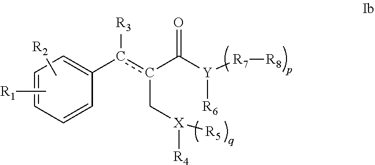

wherein

R₁, R₂, R₃, R₄, R₅, R₆, R₇, R₈, q, p and X are as defined above,

their enantiomers, diastereomers, rotamers or mixtures thereof;

and the pharmaceutically acceptable salts or solvates thereof.

According to a particularly preferred aspect the compounds for use according to the invention wherein Y is N have general formula (Ic):

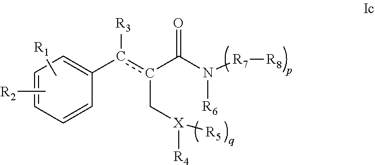

wherein

R₁, R₂, R₃, R₄, R₅, R₆, R₇, R₈, q, p and X are as defined above,

their enantiomers, diastereomers, rotamers or mixtures thereof;

and the pharmaceutically acceptable salts or solvates thereof.

According to the present invention, “C₁-C₈-alkyl” represents a linear or branched, saturated or unsaturated alkyl chain containing 1 to 8 carbon atoms. “C₁-C₄-alkyl” represents a linear or branched alkyl chain containing 1 to 4 carbon atoms, which may be saturated, such as methyl, ethyl, propyl, isopropyl, butyl, sec-butyl or tert-butyl, or unsaturated, such as ethenyl, 1-propenyl, 2-propenyl, 1-butenyl, 2-butenyl, 3-butenyl, ethynyl, 1-propynyl, 2-propynyl, 1-butynyl, 2-butynyl or 3-butynyl, preferably ethynyl, 1-propynyl, 2-propynyl, 1-butynyl, 2-butynyl or 3-butynyl. The “C₁-C₈-alkyl” or “C₁-C₄-alkyl” group can be substituted by a halogen (Cl, F, Br, I), OH, cyano group, nitro group, amino group or C₁-C₄-alkyl-amino, C₁-C₄-alkyl, C₁-C₄-alkoxy, C₁-C₄-allyl, C₁-C₄-haloalkyl, C₁-C₄-haloalkoxy.

“C₁-C₄-alkoxy” represents a linear or branched, saturated or unsaturated alkyl radical containing 1 to 4 carbon atoms, bonded to an oxygen atom. The “C₁-C₄-alkoxy” group can be substituted by C_{J}-C₄-alkyl.

“C₃-C₁₀-cycloalkyl” represents a saturated or partly saturated hydrocarbon ring containing 3 to 10 carbon atoms, which is monocyclic, preferably cyclopropyl, cyclobutyl, cyclopentyl, cyclohexyl or cycloheptyl, or bicyclic, preferably decalin or tetralin. “C₅-C₁₀-cycloalkyl” represents a saturated or partly saturated hydrocarbon ring containing 5 to 10 carbon atoms, which is monocyclic, preferably cyclopentyl, cyclohexyl or cycloheptyl, or bicyclic, preferably decalin or tetralin. “C₃-C₆-cycloalkyl” represents a saturated or partly saturated hydrocarbon ring containing 3 to 6 carbon atoms, which is monocyclic, preferably cyclopropyl, cyclobutyl, cyclopentyl, cyclohexyl or cycloheptyl. The “C₃-C₁₀-alkyl” group can be substituted by a halogen (Cl, F, Br, I), OH, cyano group, nitro group, amino group or C₁-C₄-alkyl-amino, C₁-C₄-alkyl, C₁-C₄-alkoxy, C₁-C₄-allyl, C₁-C₄-haloalkyl, C₁-C₄-haloalkoxy.

“C₆-C₁₀-aryl” represents a monocyclic or bicyclic or tricyclic aromatic ring having 6 to 10 carbon atoms, preferably monocyclic or bicyclic, and is more preferably phenyl or naphthyl; most preferably it is phenyl.

“C₆-C₁₀-aryl” represents a monocyclic or bicyclic aromatic ring having 6 to 10 carbon atoms.

“5- to 10-membered heterocycle” represents a saturated or partly saturated, monocyclic or bicyclic ring containing one or more heteroatoms selected from nitrogen, oxygen and sulfur; the heterocycle preferably contains at least one nitrogen atom.

“Monocyclic or bicyclic 5- to 10-membered heteroaryl” represents a monocyclic or polycyclic aromatic ring containing one or more heteroatoms selected from nitrogen, oxygen and sulfur. Preferably, the monocyclic heteroaryl ring contains at least one nitrogen atom. More preferably it is a monocyclic heteroaryl, for example selected from thiophene, furan, pyrrole, oxazole, isoxazole, thiadiazole, oxadiazole, imidazole and pyrimidine. Alternatively, it is a bicyclic heteroaryl, such as indole.

The bicyclic systems can be “condensed ring systems”, “bridged ring systems” or “bicyclic spiro-ring systems”.

The term “halogen” refers to fluorine, chlorine, bromine and iodine.

According to the invention, an aryl, heteroaryl or arylalkyl group, such as a phenylalkyl group, can be substituted with a halogen (Cl, F, Br, I), OH, cyano group, nitro group, amino group or C₁-C₄-alkyl-amino, C₁-C₄-alkyl, C₁-C₄-alkoxy, C₁-C₄-allyl, C₁-C₄-haloalkyl, C₁-C₄-haloalkoxy defined according to the invention.

An amido, amino or ester group can be substituted with C₁-C₄-alkyl.

According to the present invention, [symbol] represents that the two carbon atoms which join A to the carbonyl group can be bonded via a single or double bond to form a saturated or unsaturated chain. When the two carbon atoms are joined by a double bond, the substituents present on the double bond can have either the E (trans) or Z (cis) configuration. When the two carbon atoms are joined by a single bond, the substituents can have any spatial arrangement.

The compounds according to the invention which have one or more stereogenic (asymmetrical) carbon atoms can exist as stereoisomers (optical isomers), i.e. as enantiomers or diastereomers or mixtures thereof. According to the present invention, the compounds can take the form of optically pure enantiomers; pure diastereomers; mixtures of enantiomers; mixtures of diastereomers; racemic mixtures, racemates, or racemate mixtures of enantiomers. Moreover, according to the present invention, the compounds can take the form of conformational isomers or rotamers.

The amino acids are in their D or L configuration.

According to the present invention, a “protecting group” can be selected from those listed in Peter G. M. Wuts, Theodora W. Greene “Greene's Protective Groups in Organic Synthesis”, Fourth Edition, 2007 John Wiley & Sons Inc., pp. 533-646 and pp. 696-926, and Isidro-Llobet A., Alvarez M. Albericio F. “Amino Acid-Protecting Groups”, Chem. Soc. Rev. 2009, 109, 2455-2504; amino protecting groups are, for example, tert-butyl-oxy-carbonyl (Boc) or acetyl, and terminal carboxyl protecting groups are, for example, methyl, ethyl, tert-butyl or benzyl.

The compounds according to the present invention can be converted to the corresponding pharmaceutically acceptable salts by reacting with the corresponding organic or inorganic acids, or organic or inorganic bases, or with amino acids such as lysine or arginine.

Examples of pharmaceutically acceptable inorganic acids or inorganic bases are hydrochloric, hydrobromic, sulphuric, phosphoric and nitric acid, sodium hydroxide, potassium hydroxide and calcium hydroxide.

Examples of pharmaceutically acceptable organic acids or organic bases are oxalic, tartaric, maleic, succinic, citric, fumaric, acetic, methanesulphonic, benzoic, carbonic and pamoic acid, tris-(2-hydroxymethyl)-aminomethane (tromethamine) and sodium methylate.

It has now surprisingly been found that the compounds of general formula (I), (Ia), (Ib) and (Ic) as defined above are useful in the prevention and/or treatment of NLRP3 inflammasome-mediated diseases and/or disorders.

As shown in the Examples, the experiments conducted demonstrate that the compounds according to the invention possess inhibitory activity towards the NLRP3 inflammasome. Such activity makes the compounds according to the invention useful in the prevention and/or treatment of diseases and/or disorders wherein activation of the NLRP3 inflammasome contributes to the onset and/or progression of said diseases or said disorders.

The object of the present invention is the use of a compound of general formula (I), (Ia), (Ib) or (Ic) as defined above as a medicament, in particular to inhibit the NLRP3 inflammasome. Inhibiting the NLRP3 inflammasome means reducing the activity of the inflammasome, and in particular the ability of the NLRP3 inflammasome to produce IL-1beta.

According to a further aspect, the invention relates to compounds of general formula (I), and the corresponding sub-formulae (Ia), (Ib) and (Ic), as defined above, for use in the prevention and/or treatment of inflammatory, autoimmune, neurodegenerative, cardiovascular, metabolic and neoplastic diseases and/or disorders.

According to a preferred aspect, the compounds according to the invention are useful in the prevention and/or treatment of inflammation associated with autoimmune, neurodegenerative, cardiovascular, metabolic and neoplastic diseases and/or disorders.

Moreover, the compounds according to the invention are useful in the prevention and/or treatment of diseases and/or disorders, or the inflammation associated therewith, such as:
cryopyrin-associated periodic syndromes (CAPS) which comprise familial cold autoinflammatory syndrome (FCAS), Muckle-Wells syndrome (MWS) and chronic infantile neurological cutaneous and articular syndrome (CINCA), also known as neonatal-onset multisystem inflammatory disease (NOMID);
asthma, chronic or acute inflammatory arthritis, osteoarthritis, rheumatoid arthritis, acute or chronic joint disease, psoriasis, sterile corneal inflammation, systemic sclerosis, ankylosing spondylitis, sepsis, chronic inflammatory bowel diseases, irritable bowel syndrome, inflammation induced by viral infections (such as those caused by the SARS-CoV-2 (COVID-19) virus;
Alzheimer's disease, multiple sclerosis, Parkinson's disease (PD), amyotrophic lateral sclerosis (ALS) and correlated symptoms (such as gastrointestinal disorders);
cardiovascular diseases (such as hypertension, myocardial infarction, diabetic cardiomyopathy, atherosclerosis, pericarditis and ischaemia);
non-alcoholic steatohepatitis (NASH), liver disease and correlated disorders such as hepatic fibrosis;
obesity, type I/type II diabetes, kidney disease and correlated disorders (such as gastrointestinal disorders);
tumours (such as stomach cancer, head/neck cancer, lung cancer, melanoma), myelodysplastic syndromes.

The preferred compounds for use according to the invention are listed in Table 1a:

TABLE la

| | | | Pyroptosis |
|---|---|---|---|
| | | | inhibition |
| Compound | Structure | | (% ± SEM) |
| INF38s | 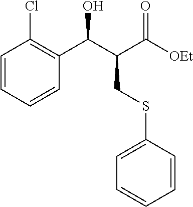 [SMILES: CCOC(=O)[C@@H](CSc1ccccc1)[C@H](O)c1ccccc1Cl] | 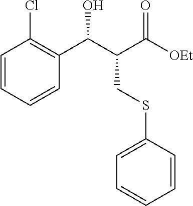 [SMILES: CCOC(=O)[C@H](CSc1ccccc1)[C@@H](O)c1ccccc1Cl] | 23.5 ± 4.2 |
| INF38a | 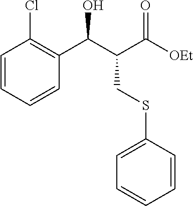 [SMILES: CCOC(=O)[C@H](CSc1ccccc1)[C@H](O)c1ccccc1Cl] | 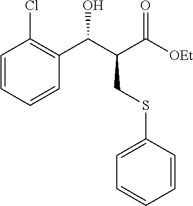 [SMILES: CCOC(=O)[C@@H](CSc1ccccc1)[C@@H](O)c1ccccc1Cl] | 26.9 ± 4.9 |
| INF44 | 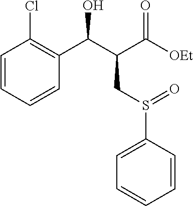 [SMILES: CCOC(=O)[C@@H](CS(=O)c1ccccc1)[C@H](O)c1ccccc1Cl] | 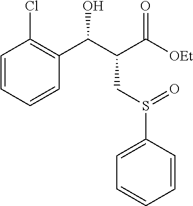 [SMILES: CCOC(=O)[C@H](CS(=O)c1ccccc1)[C@@H](O)c1ccccc1Cl] | <10 |
| INF45 | 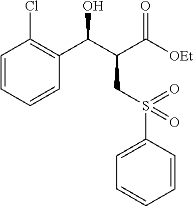 [SMILES: CCOC(=O)[C@@H](CS(=O)(=O)c1ccccc1)[C@H](O)c1ccccc1Cl] | 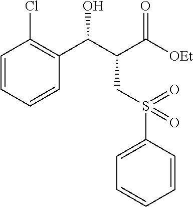 [SMILES: CCOC(=O)[C@H](CS(=O)(=O)c1ccccc1)[C@@H](O)c1ccccc1Cl] | 45.8 ± 6.7 |
| INF42 | 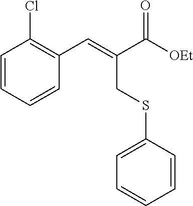 [SMILES: CCOC(=O)/C(=C/c1ccccc1Cl)CSc1ccccc1] | | 69.1 ± 2.2 |
| INF50 | 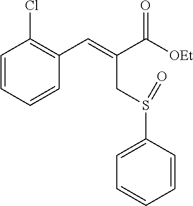 [SMILES: CCOC(=O)/C(=C/c1ccccc1Cl)CS(=O)c1ccccc1] | | 41.1 ± 2.5 |
| INF56 | 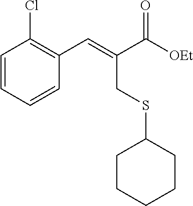 [SMILES: CCOC(=O)/C(=C/c1ccccc1Cl)CSC1CCCCC1] | | 41.6 ± 3.9 |
| INF57 | 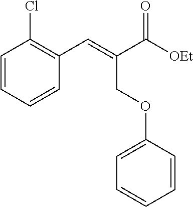 [SMILES: CCOC(=O)/C(=C/c1ccccc1Cl)COc1ccccc1] | | 34.5 ± 0.3 |
| INF43 | 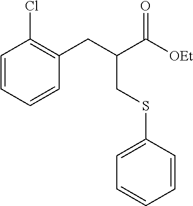 [SMILES: CCOC(=O)C(CSc1ccccc1)Cc1ccccc1Cl] | | 51.0 ± 5.6 |
| INF48 | 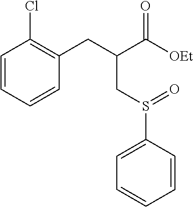 [SMILES: CCOC(=O)C(Cc1ccccc1Cl)CS(=O)c1ccccc1] | | 33.6 ± 1.2 |
| INF49 | 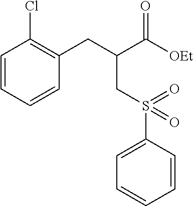 [SMILES: CCOC(=O)C(Cc1ccccc1Cl)CS(=O)(=O)c1ccccc1] | | 50.6 ± 5.8 |
| INF55 | 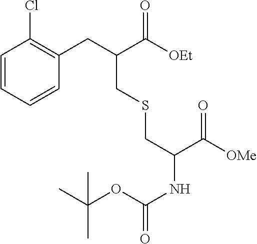 [SMILES: CCOC(=O)C(CSCC(NC(=O)OC(C)(C)C)C(=O)OC)Cc1ccccc1Cl] | | <10 |
| INF110 | 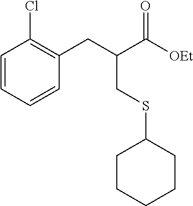 [SMILES: CCOC(=O)C(CSC1CCCCC1)Cc1ccccc1Cl] | | <10 |
| INF85 | 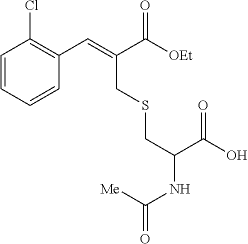 [SMILES: CCOC(=O)/C(=C/c1ccccc1Cl)CSCC(NC(C)=O)C(=O)O] | | 21.3 ± 4.6 |
| INF82 | 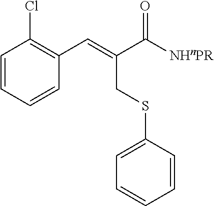 | | 19.0 ± 4.1 |
| INF80 | 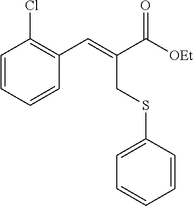 [SMILES: CCOC(=O)/C(=C/c1ccccc1Cl)CSc1ccccc1] | | <10 |
| INF86 | 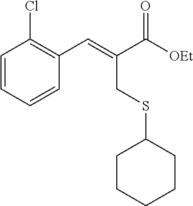 [SMILES: CCOC(=O)/C(=C/c1ccccc1Cl)CSC1CCCCC1] | | <10 |
| INF111 | 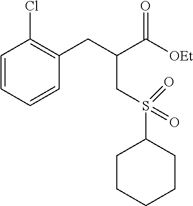 [SMILES: CCOC(=O)C(Cc1ccccc1Cl)CS(=O)(=O)C1CCCCC1] | | <10 |
| INF176 | 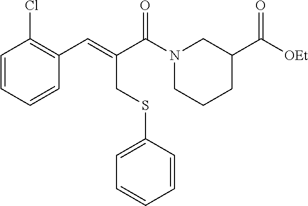 [SMILES: CCOC(=O)C1CCCN(C(=O)/C(=C/c2ccccc2Cl)CSc2ccccc2)C1] | | 45.20 ± 7.2 |
| INF202 | 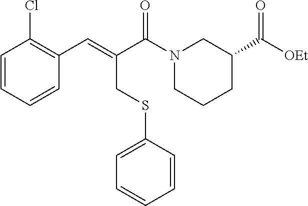 [SMILES: CCOC(=O)[C@@H]1CCCN(C(=O)/C(=C/c2ccccc2Cl)CSc2ccccc2)C1] | | 40.9 ± 1.2 |
| INF203 | 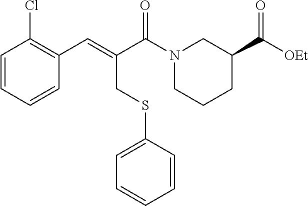 [SMILES: CCOC(=O)[C@H]1CCCN(C(=O)/C(=C/c2ccccc2Cl)CSc2ccccc2)C1] | | 14.4 ± 4.3 |
| INF177 | 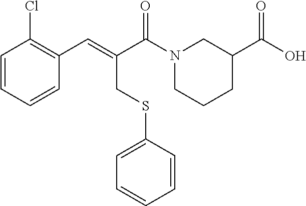 [SMILES: O=C(O)C1CCCN(C(=O)/C(=C/c2ccccc2Cl)CSc2ccccc2)C1] | | 20.1 ± 9.8 |
| INF180 | 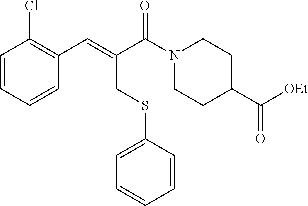 [SMILES: CCOC(=O)C1CCN(C(=O)/C(=C/c2ccccc2Cl)CSc2ccccc2)CC1] | | 13.8 ± 7.1 |
| INF184 | 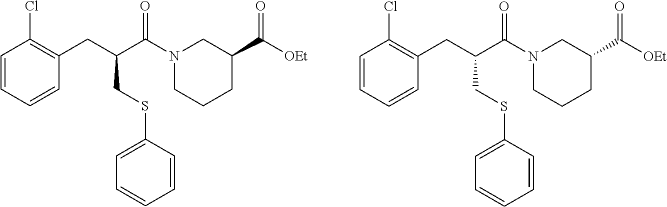 [SMILES: CCOC(=O)[C@H]1CCCN(C(=O)[C@@H](CSc2ccccc2)Cc2ccccc2Cl)C1 ; CCOC(=O)[C@@H]1CCCN(C(=O)[C@H](CSc2ccccc2)Cc2ccccc2Cl)C1] | | <10 |
| INF185 | 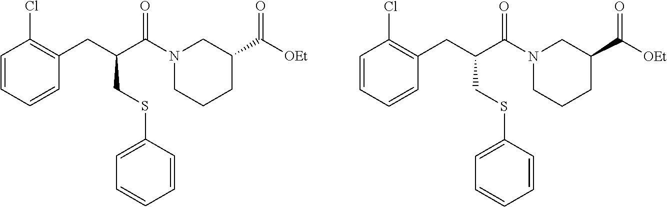 [SMILES: CCOC(=O)[C@@H]1CCCN(C(=O)[C@@H](CSc2ccccc2)Cc2ccccc2Cl)C1 ; CCOC(=O)[C@H]1CCCN(C(=O)[C@H](CSc2ccccc2)Cc2ccccc2Cl)C1] | | <10 |
| INF186 | 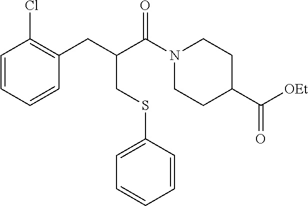 [SMILES: CCOC(=O)C1CCN(C(=O)C(CSc2ccccc2)Cc2ccccc2Cl)CC1] | | <10 |
| INF187 | 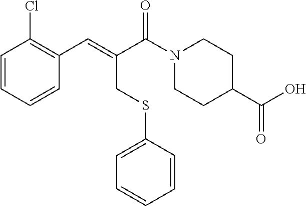 [SMILES: O=C(O)C1CCN(C(=O)/C(=C/c2ccccc2Cl)CSc2ccccc2)CC1] | | <10 |
| INF188 | 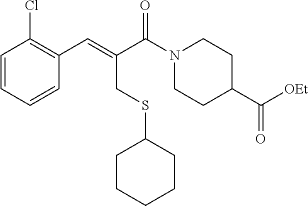 [SMILES: CCOC(=O)C1CCN(C(=O)/C(=C/c2ccccc2Cl)CSC2CCCCC2)CC1] | | <10 |
| INF192 | 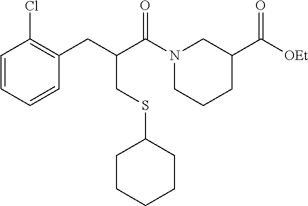 [SMILES: CCOC(=O)C1CCCN(C(=O)C(CSC2CCCCC2)Cc2ccccc2Cl)C1] | | 62.2 ± 9.78 |
| INF193 | 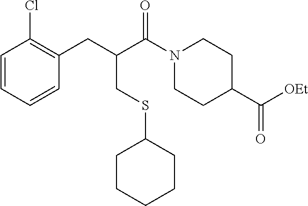 [SMILES: CCOC(=O)C1CCN(C(=O)C(CSC2CCCCC2)Cc2ccccc2Cl)CC1] | | <10 |
| INF194 | 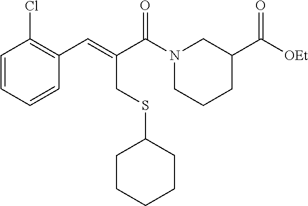 [SMILES: CCOC(=O)C1CCCN(C(=O)/C(=C/c2ccccc2Cl)CSC2CCCCC2)C1] | | <10 |
| INF61 | 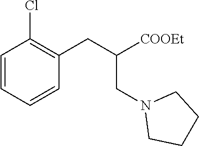 [SMILES: CCOC(=O)C(Cc1ccccc1Cl)CN1CCCC1] | | 41.7 ± 5.2 |
| INF37 syn | 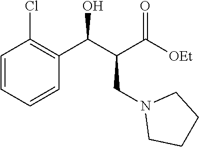 [SMILES: CCOC(=O)[C@@H](CN1CCCC1)[C@H](O)c1ccccc1Cl] | 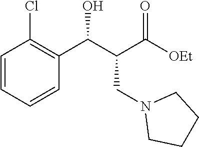 [SMILES: CCOC(=O)[C@H](CN1CCCC1)[C@@H](O)c1ccccc1Cl] | 32.2 ± 5.7 |
| INF37 anti | 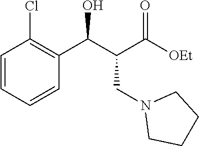 [SMILES: CCOC(=O)[C@H](CN1CCCC1)[C@H](O)c1ccccc1Cl] | 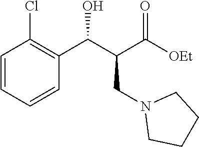 [SMILES: CCOC(=O)[C@@H](CN1CCCC1)[C@@H](O)c1ccccc1Cl] | 21.1 ± 2.9 |

| | | |
|---|---|---|
| INF219 | 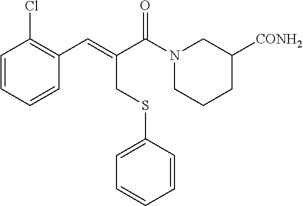 [SMILES: NC(=O)C1CCCN(C(=O)/C(=C/c2ccccc2Cl)CSc2ccccc2)C1] | 13.0 ± 8.2 |
| INF220 | 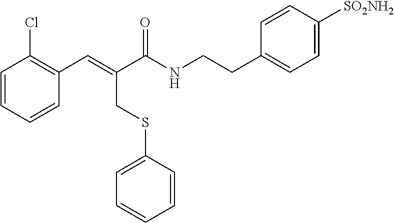 [SMILES: NS(=O)(=O)c1ccc(CCNC(=O)/C(=C/c2ccccc2Cl)CSc2ccccc2)cc1] | 73.9 ± 13.9 |
| INF51 | 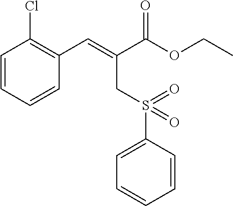 [SMILES: CCOC(=O)/C(=C/c1ccccc1Cl)CS(=O)(=O)c1ccccc1] | 38.9 ± 4.5 |
| INF230 | 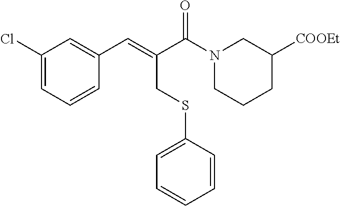 [SMILES: CCOC(=O)C1CCCN(C(=O)/C(=C/c2cccc(Cl)c2)CSc2ccccc2)C1] | 39.0 ± 15.4 |
| INF231 | 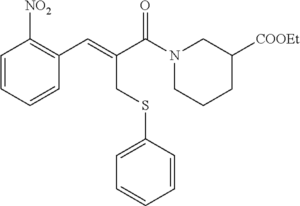 [SMILES: CCOC(=O)C1CCCN(C(=O)/C(=C/c2ccccc2[N+](=O)[O-])CSc2ccccc2)C1] | 32.6 ± 19.8 |

According to a further preferred aspect, the compounds for use according to the invention are selected from INF37syn, INF37anti, INF38s, INF38a, INF42, INF43, INF45, INF49, INF56, INF57, INF6l, INF82, INF85, INF176, INF177, INF180, INF202, INF203, INF192, INF219 and INF220, more preferably from INF43, INF49, INF56, INF57, INF61, INF85, INF176, INF177, INF192, INF219 and INF220.

Further preferred compounds according to the invention are listed in Table 1b:

TABLE 1b

| Compound | Structure |
|---|---|
| Br1 | 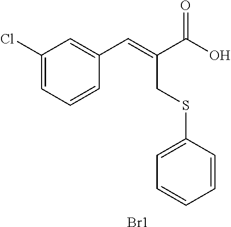 |
| Br2S | 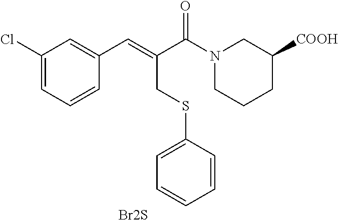 [SMILES: O=C(O)[C@H]1CCCN(C(=O)/C(=C/c2cccc(Cl)c2)CSc2ccccc2)C1 ; "Br2S"] |
| Br2R | 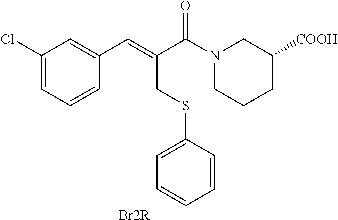 [SMILES: O=C(O)[C@@H]1CCCN(C(=O)/C(=C/c2cccc(Cl)c2)CSc2ccccc2)C1 ; "Br2R"] |
| Br4R | 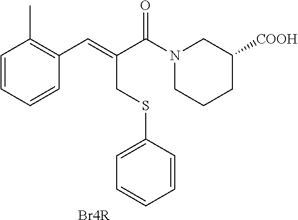 [SMILES: Cc1ccccc1/C=C(\CSc1ccccc1)C(=O)N1CCC[C@@H](C(=O)O)C1 ; "Br4R"] |
| Br7 | 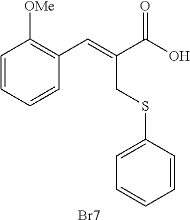 [SMILES: COc1ccccc1/C=C(\CSc1ccccc1)C(=O)O ; "Br7"] |
| Br8R | 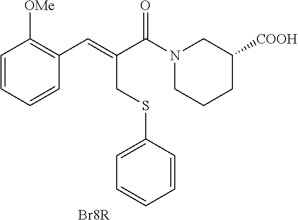 [SMILES: COc1ccccc1/C=C(\CSc1ccccc1)C(=O)N1CCC[C@@H](C(=O)O)C1 ; "Br8R"] |
| Br9R | 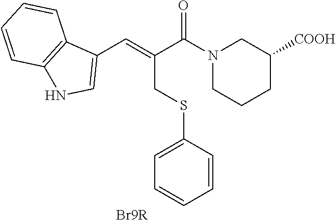 [SMILES: O=C(O)[C@@H]1CCCN(C(=O)/C(=C/c2c[nH]c3ccccc23)CSc2ccccc2)C1 ; "Br9R"] |
| Br10R | 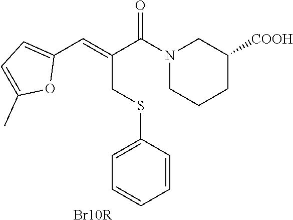 [SMILES: "Br10R" ; Cc1ccc(/C=C(\CSc2ccccc2)C(=O)N2CCC[C@@H](C(=O)O)C2)o1] |
| Br10S | 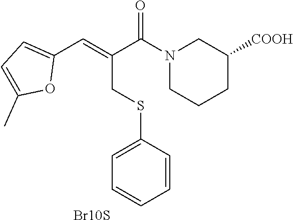 [SMILES: "Br10S" ; Cc1ccc(/C=C(\CSc2ccccc2)C(=O)N2CCC[C@@H](C(=O)O)C2)o1] |
| Br11R | 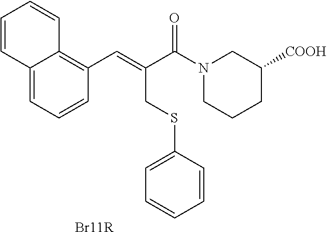 [SMILES: "Br11R" ; O=C(O)[C@@H]1CCCN(C(=O)/C(=C/c2cccc3ccccc23)CSc2ccccc2)C1] |
| Br12 | 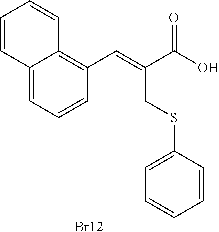 [SMILES: "Br12" ; O=C(O)/C(=C/c1cccc2ccccc12)CSc1ccccc1] |
| Br13 | 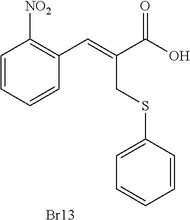 [SMILES: "Br13" ; O=C(O)/C(=C/c1ccccc1[N+](=O)[O-])CSc1ccccc1] |
| Br14R | 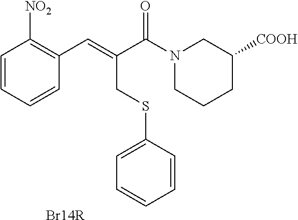 [SMILES: "Br14R" ; O=C(O)[C@@H]1CCCN(C(=O)/C(=C/c2ccccc2[N+](=O)[O-])CSc2ccccc2)C1] |
| Br14S | 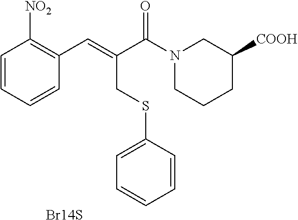 [SMILES: "Br14S" ; O=C(O)[C@H]1CCCN(C(=O)/C(=C/c2ccccc2[N+](=O)[O-])CSc2ccccc2)C1] |
| Br15 | 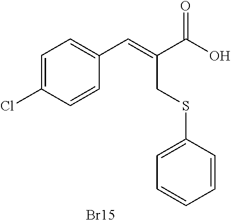 [SMILES: "Br15" ; O=C(O)/C(=C/c1ccc(Cl)cc1)CSc1ccccc1] |
| Br16R | 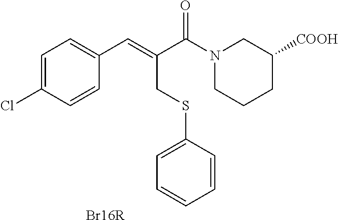 [SMILES: "Br16R" ; O=C(O)[C@@H]1CCCN(C(=O)/C(=C/c2ccc(Cl)cc2)CSc2ccccc2)C1] |
| Br17 | 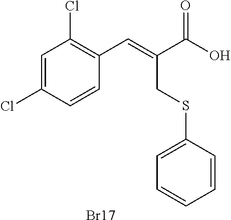 [SMILES: "Br17" ; O=C(O)/C(=C/c1ccc(Cl)cc1Cl)CSc1ccccc1] |
| Br18R | 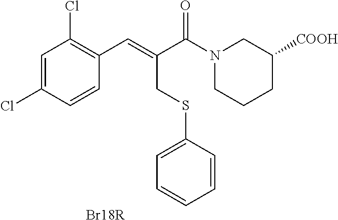 [SMILES: "Br18R" ; O=C(O)[C@@H]1CCCN(C(=O)/C(=C/c2ccc(Cl)cc2Cl)CSc2ccccc2)C1] |
| Br4S | 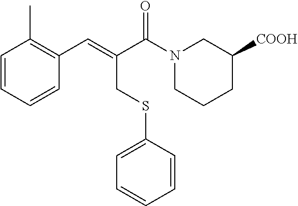 [SMILES: Cc1ccccc1/C=C(\CSc1ccccc1)C(=O)N1CCC[C@H](C(=O)O)C1] |
| Br8S | 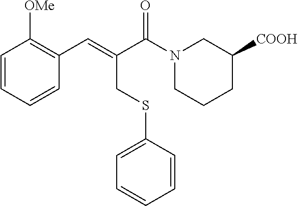 [SMILES: COc1ccccc1/C=C(\CSc1ccccc1)C(=O)N1CCC[C@H](C(=O)O)C1] |
| Br11S | 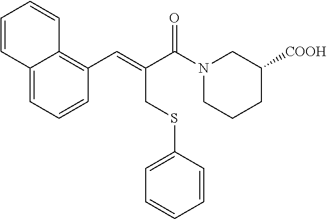 [SMILES: O=C(O)[C@@H]1CCCN(C(=O)/C(=C/c2cccc3ccccc23)CSc2ccccc2)C1] |

The compounds according to the invention are useful in treatment methods comprising administration of said compounds in a therapeutically effective amount to an individual in need thereof for the prevention and/or treatment of diseases and/or disorders as defined above.

The compounds according to the invention of formula (I) can be used in combination with other therapeutic agents such as anti-inflammatories, non-steroidal anti-inflammatory drugs (NSAIDs), biological, anti-diabetic, anti-Alzheimer, anti-Parkinson or anti-sclerosis medicaments, to achieve greater therapeutic efficacy, a reduction in the amount of medicament administered to the patient, and therefore a lower incidence of associated adverse effects.

The invention also relates to compositions containing at least one compound of general formula (I), (Ia), (Ib) or (Ic), and at least one pharmaceutically acceptable excipient or carrier.

The daily dose of active ingredient administered can be a single dose or an effective amount divided into multiple doses to be administered, for example, in the course of a day. The dosage regimen and frequency of administration for treatment of the disorders described above with the compound according to the invention and/or with the pharmaceutical compositions according to the present invention will be selected on the basis of a variety of factors including, for example, the patient's age, body weight, sex and medical conditions and the severity of the disease, the administration route, pharmacological factors, and any concomitant treatment with other medicaments. In some cases, dosage levels lower or higher than said range, and/or more frequent doses, can be used, obviously at the discretion of the doctor and depending on the stage of the disease. A preferred aspect of the invention is compounds of general formula (I):

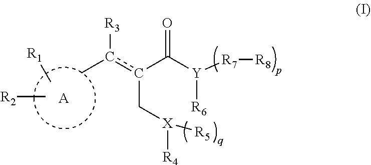

wherein:

A is a C₃-C₁₀-cycloalkyl, preferably monocyclic or bicyclic C₅-C₁₀-cycloalkyl; 5- to 10-membered, saturated or partly saturated, monocyclic or bicyclic heterocycle; monocyclic or bicyclic C₆-C₁₀-aryl; 5- to 10-membered monocyclic or bicyclic heteroaryl; A is preferably a 5 or 6-membered, saturated or partly saturated, monocyclic heterocycle, or a 9 or 10-membered, saturated or partly saturated, bicyclic heterocycle; or a monocyclic C₅-C₆-aryl, or a bicyclic C₉-C₁₀-aryl; or a 5 or 6-membered monocyclic heteroaryl or a 9 or 10-membered bicyclic heteroaryl; wherein the heteroatom is preferably N or O; more preferably A is phenyl, naphthyl, furanyl or indolyl, and most preferably A is phenyl;

R₁ and R₂, which are the same or different, can occupy any position on A, and can be hydrogen; halogen such as F, Cl, Br or I; linear or branched, substituted or unsubstituted, saturated or unsaturated C₁-C₄-alkyl; linear or branched, substituted or unsubstituted, saturated or unsaturated C₁-C₄-alkoxy; a nitro group; nitrile; a substituted or unsubstituted amido group; a substituted or unsubstituted amino group; a substituted or unsubstituted ester group; a trifluoromethyl group; R₁ and R₂ are preferably hydrogen, halogen such as F, Cl, Br or I, linear or branched C₁-C₄-alkyl, linear or branched C₁-C₄-alkoxy, a nitro group; R₁ and R₂ are more preferably hydrogen, chloro, bromo, methyl, methoxy, a nitro group; most preferably R₁ is hydrogen and R₂ is chloro;

wherein at least one of R₁ and R₂ is other than hydrogen when A is phenyl,
wherein at least one of R₁ and R₂ is other than hydrogen when X is SO₂,
wherein at least one of R₁ and R₂ is preferably other than H and is in the 2 position when A is phenyl, and R₆ and R₇ do not form a ring and the other Rj or R₂ can occupy any other position on A,
R₁ or R₂ is preferably a halogen such as F, Cl, Br or I, is in the 2 position when A is phenyl, and is more preferably Cl;

[symbol] can be a single bond or a double bond;

R₃ can be —H, —OH, —OR₉ or —O(CO)R₉, wherein R₉ can be hydrogen, a linear or branched, substituted or unsubstituted, saturated or unsaturated C₁-C₄-alkyl; R₃ is preferably hydrogen or —OH; R₃ is more preferably hydrogen;

in the

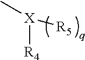

group, X can be N, O, S, S(O) or SO₂, or can be O, S, S(O) or SO₂ when Y is O;

R₄ can be a linear or branched, substituted or unsubstituted, saturated or unsaturated C₁₋₄ alkyl group; monocyclic or bicyclic C₃-C₁₀-cycloalkyl; substituted or unsubstituted, preferably a C₃-C₆-cycloalkyl; monocyclic or bicyclic C₆-C₁₄-aryl, substituted or unsubstituted, preferably a C₆-C₁₀-aryl, more preferably a C₅-C₆-aryl; 5- to 10-membered heterocycle, saturated or partly saturated, monocyclic or bicyclic, substituted or unsubstituted, preferably a C₅-C₆-heterocycle; monocyclic or polycyclic 5- to 14-membered heteroaryl, preferably monocyclic or bicyclic, substituted or unsubstituted, preferably a C₅-C₆-heteroaryl; R₄ is preferably monocyclic or bicyclic C₆-C₁₀-aryl, substituted or unsubstituted, or C₃-C₆-cycloalkyl, substituted or unsubstituted; more preferably, R₄ is cyclohexyl or phenyl;

q can be 0 (zero) or 1; when q is equal to 1, X is N and R₅ is hydrogen; a linear or branched, substituted or unsubstituted, saturated or unsaturated C₁-C₄-alkyl group; monocyclic or bicyclic C₃-C₁₀-cycloalkyl;

alternatively, the

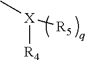

group can be an amino-acid residue wherein:
X is an N, S or O atom, or an S or O atom when Y is O, of the side chain of an amino acid, preferably natural, selected from serine; tyrosine; threonine; lysine; cysteine; q is zero (R₅ is therefore not present) and R₄ is the remainder of the amino acid which can be protected or unprotected on the NH₂ and/or COOH terminal groups; in a preferred aspect, the terminal NH₂ group is acetylated; in a preferred aspect, the amino-acid residue is N-acetylcysteine or N-Boc cysteine methyl ester; or
X is the N atom of the terminal amino group bonded to the stereogenic carbon atom in alpha of a preferably natural, protected or unprotected, amino acid, selected from alanine, arginine, asparagine, aspartic acid, cysteine, glycine, glutamic acid, glutamine, histidine, isoleucine, leucine, lysine, methionine, phenylalanine, proline, serine, threonine, tryptophan, tyrosine and valine; q is equal to 1, R₅ is hydrogen; and R₄ represents the remainder of the amino-acid structure, protected or unprotected, for example acetylated on the N atom of the side chain or esterified with a linear or branched C₁-C₄-alkyl group, preferably methyl, on the terminal carboxyl group;

alternatively when, in the

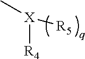

group, X is N, Y is other than O, R₄ and R₅ can be joined to form a monocyclic or bicyclic, saturated, partly saturated or unsaturated C₃-C₁₀-heterocyclic ring with the N atom; R₄ and R₅ preferably form a monocyclic C₃-C₆-heterocyclic ring with the N atom; more preferably, R₄ and R₅ form a piperidine or pyrrolidine ring with the N atom; most preferably R₄ and R₅ form a pyrrolidine ring with the N atom;

Y can be selected from O, N and S; is preferably O or N; and is more preferably N;

when Y is an oxygen or sulfur atom, in the —(R₇-R₈)p group p is equal to zero and R₆ can be hydrogen, a linear or branched, substituted or unsubstituted, saturated or unsaturated C₁-C₈-alkyl group; a monocyclic or bicyclic C₃-C₁₀-cycloalkyl; a substituted or unsubstituted arylalkyl; a 6- to 14-membered monocyclic or bicyclic heteroaryl; R₆ is preferably hydrogen or a linear or branched, substituted or unsubstituted, saturated or unsaturated C₁-C₄-alkyl group; R₆ is more preferably a linear or branched, saturated, unsubstituted C₁-C₄-alkyl group; R₆ is most preferably ethyl;

when Y is a nitrogen atom, p is equal to 1, R₆ and R₇, which are the same or different, are selected from hydrogen, a linear or branched, saturated or unsaturated, substituted or unsubstituted C₁-C₄-alkyl group; a substituted or unsubstituted aryl, arylalkyl or heteroaryl group; they can preferably be a substituted phenylalkyl group; more preferably —(CH₂)₂-phenyl-SO₂NH₂; most preferably R₆ is hydrogen and R₇ is —(CH₂)₂-phenyl-SO₂NH₂;

R₈ can be selected from H, COOH, COOR₉, C(O)R₉, CN, CONH(R₉), S(O)NHR₉ and S(O)₂NHR₉, wherein R₉ is as defined above;

alternatively, R₆ and R₇ can be joined to form a 3- to 8-membered heterocyclic ring;

R₈ is as defined above;

and their enantiomers, diastereomers, rotamers or mixtures thereof;

and the pharmaceutically acceptable salts or solvates thereof;

According to one aspect of the invention, in the compounds of formula (I), when

R₆ and R₇ do not form a ring, in the compounds of formula (Ib) and formula (Ic) as defined below:
when A is phenyl, R₁ and R₂ are as defined above, [symbol] is a double bond, R₃ is H or OH, Y is O, X is S, q and p are zero, R₄ is methyl, R₆ is hydrogen, a linear or branched, substituted or unsubstituted, saturated or unsaturated C₃-C₈-alkyl group; a monocyclic or bicyclic C₃-C₁₀-cycloalkyl; a substituted or unsubstituted arylalkyl; a 6- to 14-membered monocyclic or bicyclic heteroaryl; R₆ is preferably hydrogen or a linear or branched, substituted or unsubstituted, saturated or unsaturated C₃-C₆-alkyl group; and/or
when A is phenyl, naphthyl or thiophene, R₁ and R₂ are as defined above, R₃ is H, Y is O, X is SO₂, q and p are zero, R₄ is phenyl, ethyl or methyl, 4-chlorophenyl, 4-toluene, R₆ is methyl or ethyl, [symbol] is a single bond; and/or
when A is phenyl or naphthyl, R₁ and R₂ are as defined above, [symbol] is a double bond, R₃ is H, Y is O, X is O, q and p are zero, R₄ is methyl, R₆ is hydrogen, methyl, a linear or branched, substituted or unsubstituted, saturated or unsaturated C₃-C₈-alkyl group; a monocyclic or bicyclic C₃-C₁₀-cycloalkyl; a substituted or unsubstituted arylalkyl; a 6- to 14-membered monocyclic or bicyclic heteroaryl; R₆ is preferably hydrogen or a linear or branched, substituted or unsubstituted, saturated or unsaturated C₃-C₆-alkyl group;
when A is phenyl, R¹ and R₂ are as defined above, [symbol] is a double bond, R₃ is H, Y is O, X is O, q and p are zero, R₄ is phenyl, R₆ is methyl, ethyl, a linear or branched, substituted or unsubstituted, saturated or unsaturated C₃-C₈-alkyl group; a monocyclic or bicyclic C₃-C₁₀-cycloalkyl; a substituted or unsubstituted arylalkyl; a 6- to 14-membered monocyclic or bicyclic heteroaryl; R₆ is preferably hydrogen or a linear or branched, substituted or unsubstituted, saturated or unsaturated C₃-C₆-alkyl group;
when A is phenyl, R₁ and R₂ are as defined above, Y is O and X is N, p is zero, R₆ is methyl, q is one, [symbol] is preferably a double bond, and is more preferably a single bond or a double bond; R₄ and R₅ are joined to form a monocyclic or bicyclic, saturated, partly saturated or unsaturated C₃-C₁₀-heterocyclic ring with the N atom; R₄ and R₅ preferably form a monocyclic C₃-C₆-heterocyclic ring with the N atom; more preferably, R₄ and R₅ form a piperidine or pyrrolidine ring with the N atom; most preferably R₄ and R₅ form a pyrrolidine ring with the N atom.

According to a further aspect of the invention:
when A is phenyl, [symbol] is a double bond, R₃ is hydrogen, Y is O, R₆ is methyl, q and p are zero and X is sulfur, R₄ is other than methyl, ethyl, n-propyl, tert-butyl, cyclohexyl, phenyl, 4-chlorophenyl, 4-bromophenyl, 3-chlorophenyl, 3-bromophenyl, 2-bromophenyl, benzyl;
when A is 4-methoxyphenyl, [symbol] is a double bond, R₃ is hydrogen, Y is O, R₆ is methyl, q and p are zero and X is sulfur, R₄ is other than methyl, ethyl, phenyl;
when A is 2-chlorophenyl, [symbol] is a double bond, R₃ is hydrogen, Y is O, R₆ is methyl, q and p are zero and X is sulfur, R₄ is other than phenyl;
when A is 3-chlorophenyl, [symbol] is a double bond, R₃ is hydrogen, Y is O, R₆ is methyl, q and p are zero and X is sulfur, R₄ is other than methyl;
when A is 4-chlorophenyl, [symbol] is a double bond, R₃ is hydrogen, Y is O, R₆ is methyl, q and p are zero and X is sulfur, R₄ is other than methyl, ethyl, phenyl, 4-chlorophenyl, 4-bromophenyl, 3-chlorophenyl, 3-bromophenyl, 2-bromophenyl, benzyl;
when A is 4-methylphenyl, [symbol] is a double bond, R₃ is hydrogen, Y is O, R₆ is methyl, q and p are zero and X is sulfur, R₄ is other than methyl, phenyl, 4-chlorophenyl, 4-bromophenyl, 3-chlorophenyl, 3-bromophenyl;
when A is 2-nitrophenyl, [symbol] is a double bond, R₃ is hydrogen, Y is O, R₆ is methyl, q and p are zero and X is sulfur, R₄ is other than methyl and phenyl;
when A is 4-isopropyl-phenyl or 4-methoxycarbonylphenyl, [symbol] is a double bond, R₃ is hydrogen, Y is O, R₆ is tert-butyl, q and p are zero and X is sulfur, R₄ is other than phenyl;
when A is 4-methoxyphenyl, [symbol] is a double bond, R₃ is hydrogen, Y is O, R₆ is hydrogen, q and p are zero and X is sulfur, R₄ is other than phenyl;
when A is 2-chlorophenyl, 4-nitrophenyl, 3-bromo-phenyl, 4-methyl-phenyl or 2-thienyl, [symbol] is a double bond, R₃ is hydrogen, Y is O, R₆ is methyl or ethyl, q and p are zero and X is SO₂, R₄ is other than phenyl;
when A is phenyl, 4-chlorophenyl, 4-methoxyphenyl, 3.4-dimethoxyphenyl, 3-methylphenyl, 1-naphthyl, 2-furyl or 4-bromo-phenyl, [symbol] is a double bond, R₃ is hydrogen, Y is O, X is SO₂, R₄ is phenyl and q and p are zero, R₆ is other than methyl;
when A is phenyl, [symbol] is a double bond, R₃ is hydrogen, Y is O, X is SO₂, R₄ is 4-methylphenyl and q and p are zero, R₆ is other than methyl;
when A is 4-bromophenyl, [symbol] is a double bond, R₃ is hydrogen, Y is O, X is SO₂, R₄ is 4-methylphenyl, 4-chlorophenyl, methyl or ethyl, q and p are zero, R₆ is other than methyl;
when A is 4-bromophenyl, [symbol] is a double bond, R₃ is hydrogen, Y is O, X is S, R₄ is ethyl, q and p are zero, R₆ is other than methyl;
when A is 4-fluorophenyl, 4-trifluoromethyl-phenyl, 4-cyanophenyl or 2,4-dichlorophenyl, [symbol] is a double bond, R₃ is hydrogen, Y is O, X is SO₂, R₄ is phenyl and q and p are zero, R₆ is other than ethyl.

When R₆ and R₇ form a ring, the compounds have formula (Ia):

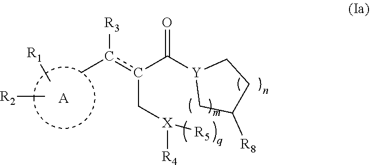

wherein

A, R₁, R₂, R₃, R₄, R₅, R₈, q, and X are as defined above;

n and m, which are the same or different, can be 0 (zero) or an integer between 1 and 3; when n and m are equal to zero, a three-membered cycle is generated between C—R₈, Y, and the remaining —CH₂— group; when n and m, which are different from one another, are 0 (zero) or 1, a 4-membered ring is formed; or n and m, which are the same or different, can be 1, 2 or 3 forming 5- to 9-, preferably 5- to 8-membered rings; according to a preferred aspect, m is 2 and n is 1; according to a more preferred aspect, n is 2 and m can be 1 or 2; most preferably, n is 2 and m is 1;

preferably, when Y is N, R₆ and R₇ can be joined to form a 3- to 6-membered monocyclic substituted heterocyclic ring with the N atom; more preferably, R₆ and R₇ form a substituted piperidine or pyrrolidine ring with the N atom; most preferably, R₆ and R₇ form, with the N atom, a piperidine ring substituted in the 3 or 4 position.

A preferred aspect of the invention is compounds of general formula (Ib):

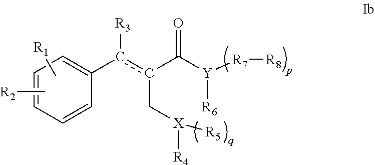

wherein

R₁, R₂, R₃, R₄, R₅, R₆, R₇, R₈, q, p, X and Y are as defined above,

their enantiomers, diastereomers, rotamers or mixtures thereof;

and the pharmaceutically acceptable salts or solvates thereof.

Particularly preferred are compounds wherein Y is N of general formula (Ic):

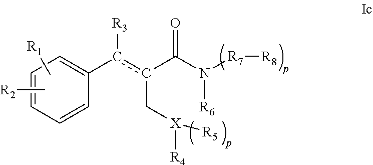

wherein

R₁, R₂, R₃, R₄, R₅, R₆, R₇, R₈, q, p and X are as defined above,

their enantiomners, diastereomers, rotamers or mixtures thereof;

and the pharmaceutically acceptable salts or solvates thereof.

The compounds of general formula (I), (Ia), (Ib) or (Ic) are preferably selected from those listed in Table 2a:

TABLE 2a

| Compound | Structure | |
|---|---|---|
| INF38s | 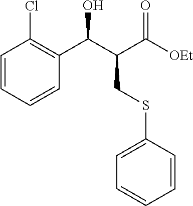 [SMILES: CCOC(=O)[C@@H](CSc1ccccc1)[C@H](O)c1ccccc1Cl] | 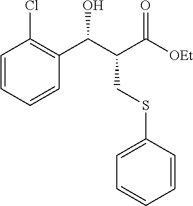 [SMILES: CCOC(=O)[C@H](CSc1ccccc1)[C@@H](O)c1ccccc1Cl] |
| INF38a | 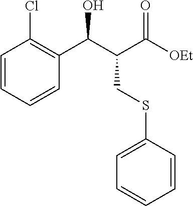 [SMILES: CCOC(=O)[C@H](CSc1ccccc1)[C@H](O)c1ccccc1Cl] | 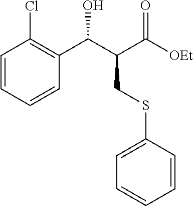 [SMILES: CCOC(=O)[C@@H](CSc1ccccc1)[C@@H](O)c1ccccc1Cl] |
| INF44 | 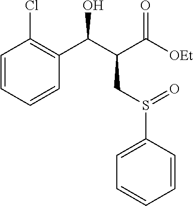 [SMILES: CCOC(=O)[C@@H](CS(=O)c1ccccc1)[C@H](O)c1ccccc1Cl] | 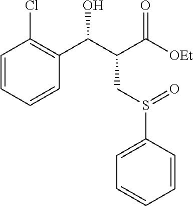 [SMILES: CCOC(=O)[C@H](CS(=O)c1ccccc1)[C@@H](O)c1ccccc1Cl] |
| INF45 | 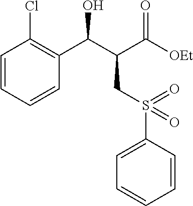 [SMILES: CCOC(=O)[C@@H](CS(=O)(=O)c1ccccc1)[C@H](O)c1ccccc1Cl] | 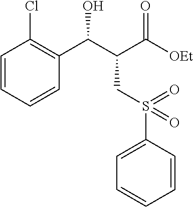 [SMILES: CCOC(=O)[C@H](CS(=O)(=O)c1ccccc1)[C@@H](O)c1ccccc1Cl] |
| INF42 | 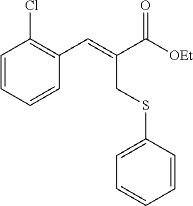 [SMILES: CCOC(=O)/C(=C/c1ccccc1Cl)CSc1ccccc1] | |
| INF50 | 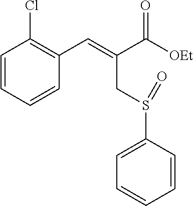 [SMILES: CCOC(=O)/C(=C/c1ccccc1Cl)CS(=O)c1ccccc1] | |
| INF56 | 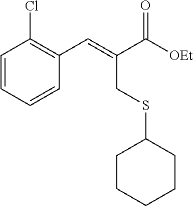 [SMILES: CCOC(=O)/C(=C/c1ccccc1Cl)CSC1CCCCC1] | |
| INF57 | 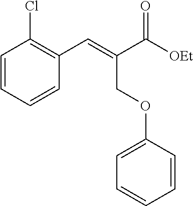 [SMILES: CCOC(=O)/C(=C/c1ccccc1Cl)COc1ccccc1] | |
| INF43 | 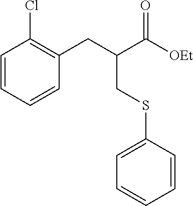 [SMILES: CCOC(=O)C(CSc1ccccc1)Cc1ccccc1Cl] | |
| INF48 | 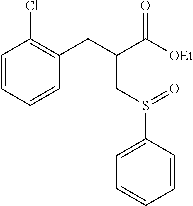 [SMILES: CCOC(=O)C(Cc1ccccc1Cl)CS(=O)c1ccccc1] | |
| INF49 | 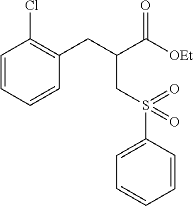 [SMILES: CCOC(=O)C(Cc1ccccc1Cl)CS(=O)(=O)c1ccccc1] | |
| INF55 | 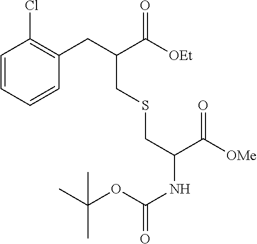 [SMILES: CCOC(=O)C(CSCC(NC(=O)OC(C)(C)C)C(=O)OC)Cc1ccccc1Cl] | |
| INF110 | 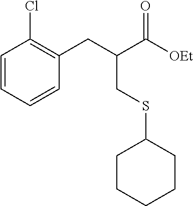 [SMILES: CCOC(=O)C(CSC1CCCCC1)Cc1ccccc1Cl] | |
| INF85 | 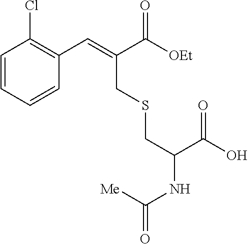 [SMILES: CCOC(=O)/C(=C/c1ccccc1Cl)CSCC(NC(C)=O)C(=O)O] | |
| INF82 | 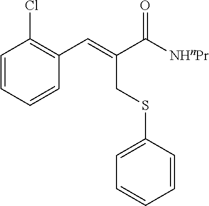 | |
| INF80 | 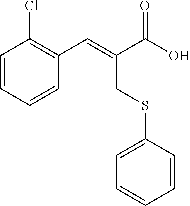 [SMILES: O=C(O)/C(=C/c1ccccc1Cl)CSc1ccccc1] | |
| INF86 | 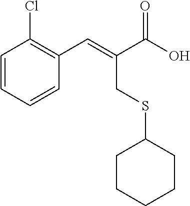 [SMILES: O=C(O)/C(=C/c1ccccc1Cl)CSC1CCCCC1] | |
| INF111 | 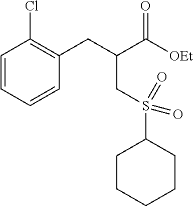 [SMILES: CCOC(=O)C(Cc1ccccc1Cl)CS(=O)(=O)C1CCCCC1] | |
| INF176 | 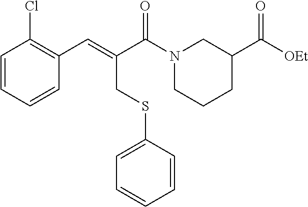 [SMILES: CCOC(=O)C1CCCN(C(=O)/C(=C/c2ccccc2Cl)CSc2ccccc2)C1] | |
| INF202 | 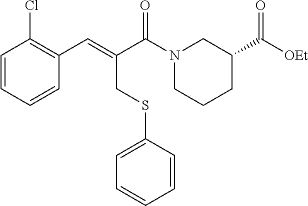 [SMILES: CCOC(=O)[C@@H]1CCCN(C(=O)/C(=C/c2ccccc2Cl)CSc2ccccc2)C1] | |
| INF203 | 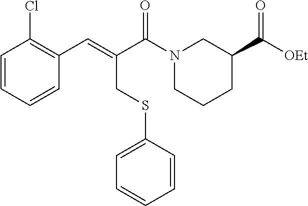 [SMILES: CCOC(=O)[C@H]1CCCN(C(=O)/C(=C/c2ccccc2Cl)CSc2ccccc2)C1] | |
| INF177 | 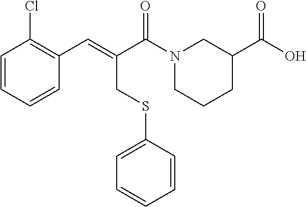 [SMILES: O=C(O)C1CCCN(C(=O)/C(=C/c2ccccc2Cl)CSc2ccccc2)C1] | |
| INF180 | 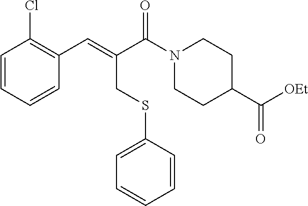 [SMILES: CCOC(=O)C1CCN(C(=O)/C(=C/c2ccccc2Cl)CSc2ccccc2)CC1] | |
| INF184 | 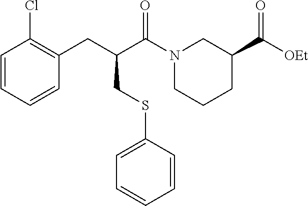 [SMILES: CCOC(=O)[C@H]1CCCN(C(=O)[C@@H](CSc2ccccc2)Cc2ccccc2Cl)C1] | 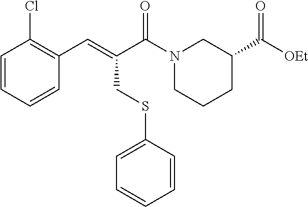 |
| INF185 | 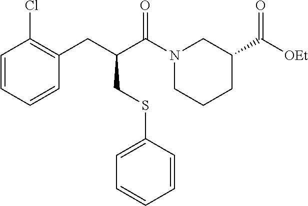 [SMILES: CCOC(=O)[C@@H]1CCCN(C(=O)[C@@H](CSc2ccccc2)Cc2ccccc2Cl)C1] | 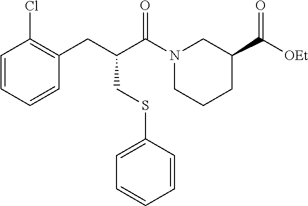 [SMILES: CCOC(=O)[C@H]1CCCN(C(=O)[C@H](CSc2ccccc2)Cc2ccccc2Cl)C1] |

| | |
|---|---|
| INF186 | 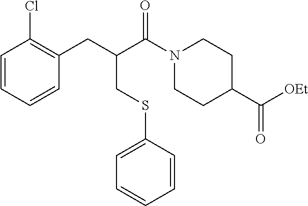 [SMILES: CCOC(=O)C1CCN(C(=O)C(CSc2ccccc2)Cc2ccccc2Cl)CC1] |
| INF187 | 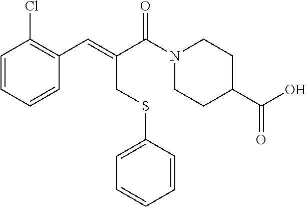 [SMILES: O=C(O)C1CCN(C(=O)/C(=C/c2ccccc2Cl)CSc2ccccc2)CC1] |
| INF188 | 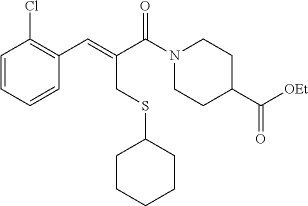 [SMILES: CCOC(=O)C1CCN(C(=O)/C(=C/c2ccccc2Cl)CSC2CCCCC2)CC1] |
| INF192 | 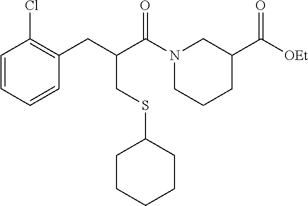 [SMILES: CCOC(=O)C1CCCN(C(=O)C(CSC2CCCCC2)Cc2ccccc2Cl)C1] |
| INF193 | 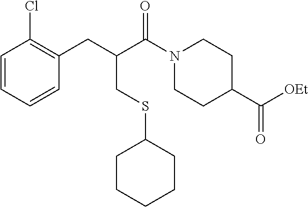 [SMILES: CCOC(=O)C1CCN(C(=O)C(CSC2CCCCC2)Cc2ccccc2Cl)CC1] |
| INF194 | 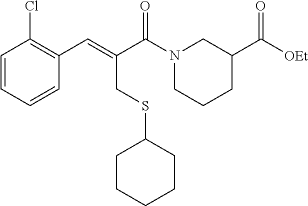 [SMILES: CCOC(=O)C1CCCN(C(=O)/C(=C/c2ccccc2Cl)CSC2CCCCC2)C1] |
| INF219 | 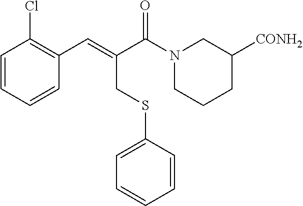 [SMILES: NC(=O)C1CCCN(C(=O)/C(=C/c2ccccc2Cl)CSc2ccccc2)C1] |
| INF220 | 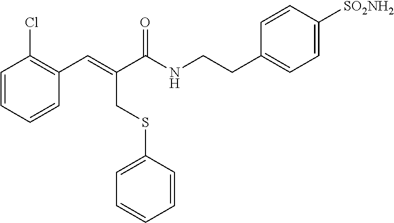 [SMILES: NS(=O)(=O)c1ccc(CCNC(=O)/C(=C/c2ccccc2Cl)CSc2ccccc2)cc1] |
| INF230 | 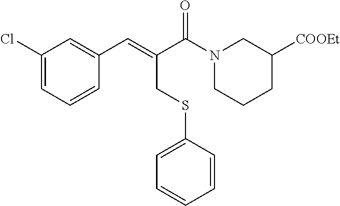 [SMILES: CCOC(=O)C1CCCN(C(=O)/C(=C/c2cccc(Cl)c2)CSc2ccccc2)C1] |
| INF231 | 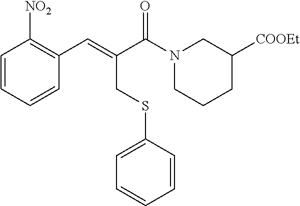 [SMILES: CCOC(=O)C1CCCN(C(=O)/C(=C/c2ccccc2[N+](=O)[O-])CSc2ccccc2)C1] |

According to a further preferred aspect, the compounds according to the invention are selected from INF38s, INF38a, INF42, INF43, INF45, INF49, INF56, INF57, INF82, INF85, INF176, INF177, INF180, INF202, INF203, INF192, INF219 and INF220, more preferably from INF43, INF49, INF56, INF57, INF85, INF176, INF177, INF192, INF219 and INF220.

Further preferred compounds according to the invention are listed in Table 2b.

TABLE 2b

| Compound | Structure |
|---|---|
| Br1 | 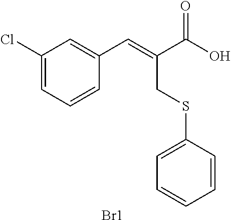 [SMILES: O=C(O)/C(=C/c1cccc(Cl)c1)CSc1ccccc1 ; "Br1"] |
| Br2S | 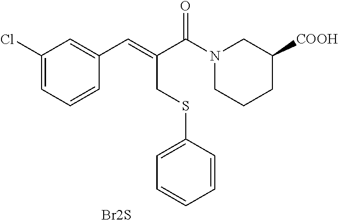 [SMILES: O=C(O)[C@H]1CCCN(C(=O)/C(=C/c2cccc(Cl)c2)CSc2ccccc2)C1 ; "Br2S"] |
| Br2R | 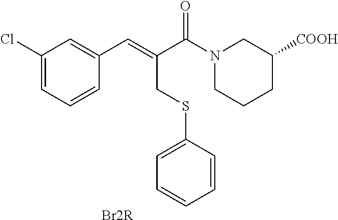 [SMILES: O=C(O)[C@@H]1CCCN(C(=O)/C(=C/c2cccc(Cl)c2)CSc2ccccc2)C1 ; "Br2R"] |
| Br4R | 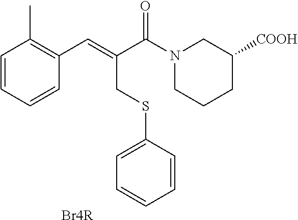 [SMILES: Cc1ccccc1/C=C(\CSc1ccccc1)C(=O)N1CCC[C@@H](C(=O)O)C1 ; "Br4R"] |
| Br7 | 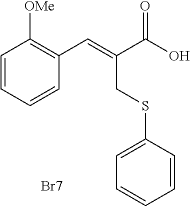 [SMILES: "Br7" ; COc1ccccc1/C=C(\CSc1ccccc1)C(=O)O] |
| Br8R | 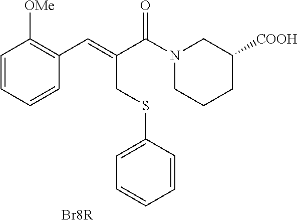 [SMILES: "Br8R" ; COc1ccccc1/C=C(\CSc1ccccc1)C(=O)N1CCC[C@@H](C(=O)O)C1] |
| Br9R | 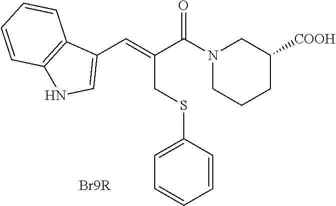 [SMILES: O=C(O)[C@@H]1CCCN(C(=O)/C(=C/c2c[nH]c3ccccc23)CSc2ccccc2)C1 ; "Br9R"] |
| Br10R | 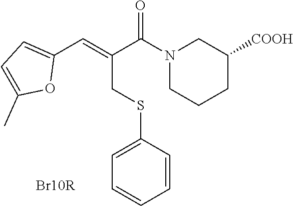 [SMILES: "Br10R" ; Cc1ccc(/C=C(\CSc2ccccc2)C(=O)N2CCC[C@@H](C(=O)O)C2)o1] |
| Br10S | 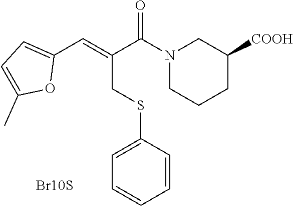 [SMILES: "Br10S" ; Cc1ccc(/C=C(\CSc2ccccc2)C(=O)N2CCC[C@H](C(=O)O)C2)o1] |
| Br11R | 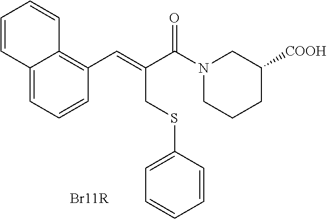 [SMILES: "Br11R" ; O=C(O)[C@@H]1CCCN(C(=O)/C(=C/c2cccc3ccccc23)CSc2ccccc2)C1] |
| Br12 | 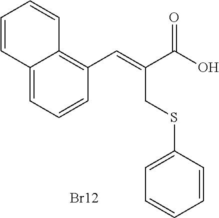 [SMILES: "Br12" ; O=C(O)/C(=C/c1cccc2ccccc12)CSc1ccccc1] |
| Br13 | 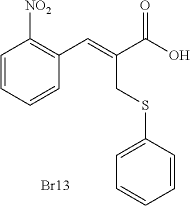 [SMILES: "Br13" ; O=C(O)/C(=C/c1ccccc1[N+](=O)[O-])CSc1ccccc1] |
| Br14R | 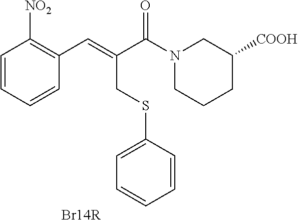 [SMILES: "Br14R" ; O=C(O)[C@@H]1CCCN(C(=O)/C(=C/c2ccccc2[N+](=O)[O-])CSc2ccccc2)C1] |
| Br14S | 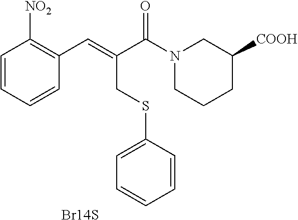 [SMILES: "Br14S" ; O=C(O)[C@H]1CCCN(C(=O)/C(=C/c2ccccc2[N+](=O)[O-])CSc2ccccc2)C1] |
| Br15 | 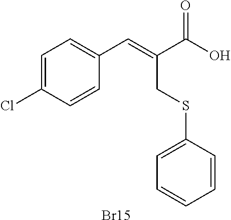 [SMILES: "Br15" ; O=C(O)/C(=C/c1ccc(Cl)cc1)CSc1ccccc1] |
| Br16R | 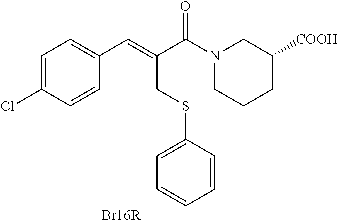 [SMILES: "Br16R" ; O=C(O)[C@@H]1CCCN(C(=O)/C(=C/c2ccc(Cl)cc2)CSc2ccccc2)C1] |
| Br17 | 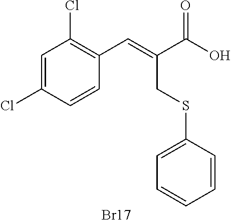 [SMILES: "Br17" ; O=C(O)/C(=C/c1ccc(Cl)cc1Cl)CSc1ccccc1] |
| Br18R | 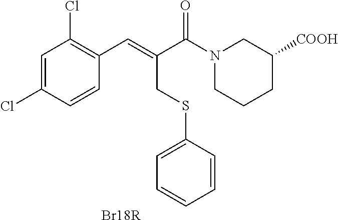 [SMILES: "Br18R" ; O=C(O)[C@@H]1CCCN(C(=O)/C(=C/c2ccc(Cl)cc2Cl)CSc2ccccc2)C1] |
| Br4S | 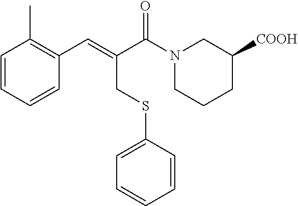 [SMILES: Cc1ccccc1/C=C(\CSc1ccccc1)C(=O)N1CCC[C@H](C(=O)O)C1] |
| Br8S | 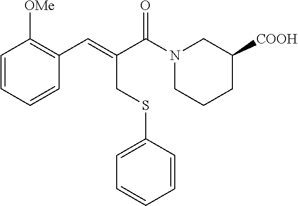 [SMILES: COc1ccccc1/C=C(\CSc1ccccc1)C(=O)N1CCC[C@H](C(=O)O)C1] |
| Br11S | 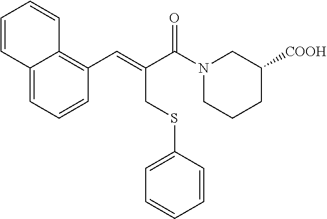 [SMILES: O=C(O)[C@@H]1CCCN(C(=O)/C(=C/c2cccc3ccccc23)CSc2ccccc2)C1] |

The compounds according to the invention can be administered orally, parenterally, topically or by injection, for example by intra-articular injection.

The compounds according to the present invention can be obtained as described in the synthesis schemes set out below.

The compounds of general formula (I) can be synthesised according to Schemes 1-7 below.

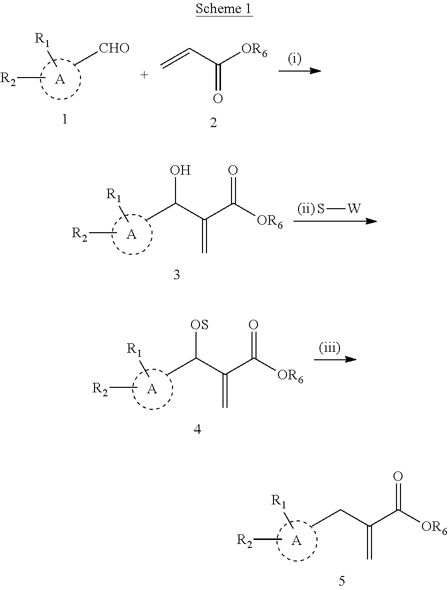

The reaction required to obtain the compounds of formula 3 can be conducted with a suitably substituted aldehyde (1), which is reacted according to a Morita-Baylis-Hillman 5 (MBH) reaction using one of the procedures reported in the literature, such as in Min Shi, Fei-Jun Wang, Mei-Xin Zhao and Yin Wei “The Chemistry of the Morita-Baylis-Hillman Reaction”, RSC Catalysis Series No. 8, 2011, Published by the Royal Society of Chemistry. In step (i), a compound of formula 1 is reacted with an α,β-unsaturated ester (2) in the presence of a base such as 1,4-diazabicyclo [2.2.2] octane (DABCO), 1,8-diazabicyclo[5.4.0]undec-7-ene (DBU), triethylamine (Et₃N) or diisopropylethylamine (DIPEA), or in the presence of a phosphine such as triphenylphosphine, tritolylphosphine or tributylphosphine in a solvent selected from acetonitrile, tetrahydrofuran (THF), dichloromethane, methanol, ethanol, 2-propanol, butanol, water or mixtures thereof, at a temperature ranging between −40° C. and +200° C. for a time ranging between a few minutes 15 and 30 days, as indicated in Scheme 1, to obtain the intermediates of formula 3.

The intermediate of formula 3 is then converted, in step (ii), to compounds of formula 4, by treatment with an electrophilic agent S—W, wherein W is a leaving group as defined in Smith M. B. and March J. “Advanced Organic Chemistry” 5^{th} ed. 2001, Wiley & Sons, p. 449. By way of example, electrophilic agents such as acetic anhydride, trifluoroacetic anhydride and trifluoromethanesulphonic anhydride can be used. The reaction is conducted in a solvent such as dichloromethane, THF or 1,4-dioxane in the presence or absence of a base for a time ranging between a few minutes and 24 hours. The preferred bases for the reaction are 4-dimethylaminopyridine (DMAP) or DBU. Compound 4 is then converted (step iii) to compound 5 by reduction with a reducing agent such as sodium borohydride, sodium cyanoborohydride or sodium triacetoxyborohydride in a solvent such as THF, diethyl ether, methanol, water or mixtures thereof. A catalyst such as DABCO can be added to the reaction mixture. The reaction is conducted at temperatures ranging between −20 and +100° C. for a time ranging between a few minutes and 24 hours.

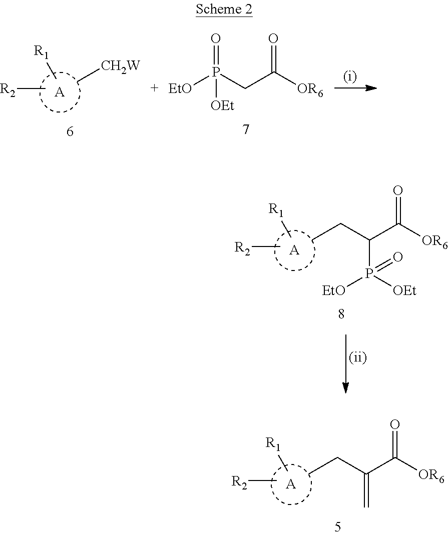

Alternatively, the compounds of formula 5 can be obtained, as described in Scheme 2, by reacting a compound of formula 6, wherein W is a leaving group as defined in Smith M. B. and March J. “Advanced Organic Chemistry” 5th ed. 2001, Wiley & Sons, p. 449. W is preferably represented by a halogen atom. The compound of formula 6 is reacted with a suitably substituted phosphonoacetate (7) in the presence of a base such as sodium hydride, sodium amide or potassium tert-butoxide, in a suitable solvent such as dimethylformamide (DMF), dimethylsulphoxide (DMSO), THF or 1,4-dioxane, at a temperature ranging between −40° C. and +200° C. for a time ranging between a few minutes and 72 hours. The resulting intermediate 8 is purified by silica gel chromatography and reacted with an excess of paraformaldehyde in the presence of a base such as K₂CO₃. Na₂CO₃ or Cs₂CO₃ in water at a temperature ranging between 0 and +100° C. for a time ranging between a few minutes and 72 hours to obtain the compound of formula 5.

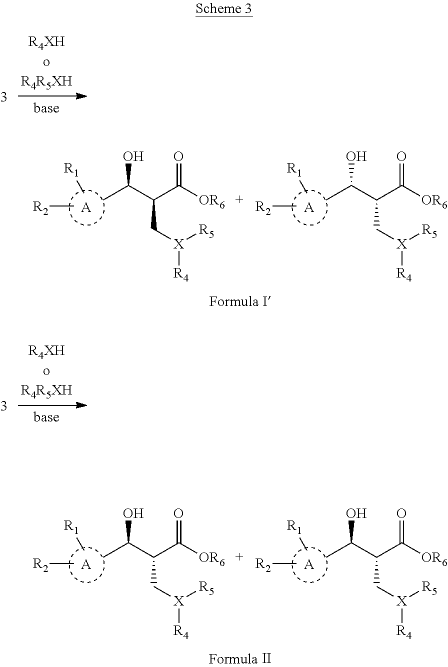

Compound 3 can be converted to the products of formulae I′ and II by reaction with a suitable nucleophile R₄XH or R₄R₅XH in a basic medium, as illustrated in Scheme 3. The reaction is conducted in a solvent such as THF, dichloromethane, acetonitrile, DMF or DMSO in the presence of a base such as DABCO, DBU, Et₃N, DIPEA, potassium tert-butoxide or sodium hydride, and in an inert gas atmosphere such as nitrogen or argon. The reaction is conducted at a temperature ranging between −20 and +180° C., preferably at room temperature. The reaction provides a mixture of products which can contain various stereoisomeric forms of the desired products. The products of formulae I′ and II are separated by preparative silica gel chromatography.

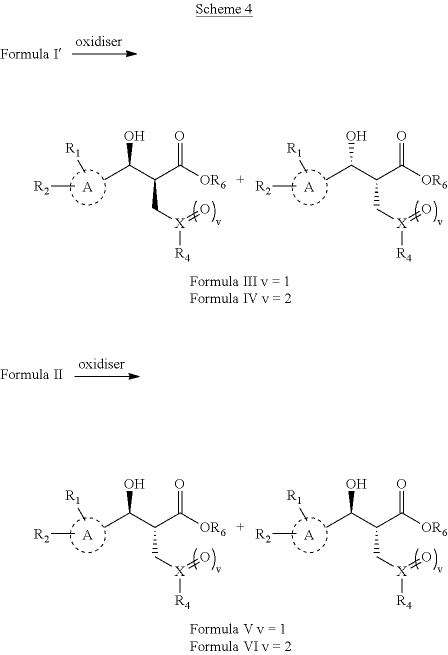

The compounds of formulae I′ and II can be oxidised using suitable oxidation reagents known to the skilled person, such as those described in Burke S. D and Danheiser R. L. eds. “Handbook of Reagents for Organic Synthesis—Oxidizing and Reducing Agents”, John Wiley and Sons Ltd 1999 pages 15-518 and the references cited. Of the preferred reagents, meta-chloroperoxybenzoic acid (mCPBA), hydrogen peroxide, ammonium persulphate and potassium peroxymonosulphate (oxone) can be used in an organic solvent such as dichloromethane or acetic acid, or in water or in mixtures thereof. The reaction times can range from a few minutes to 72 hours. The reaction can be conducted at temperatures ranging between −78 and +180° C. Compounds having formulae III-VI are thus obtained, after chromatographic purification.

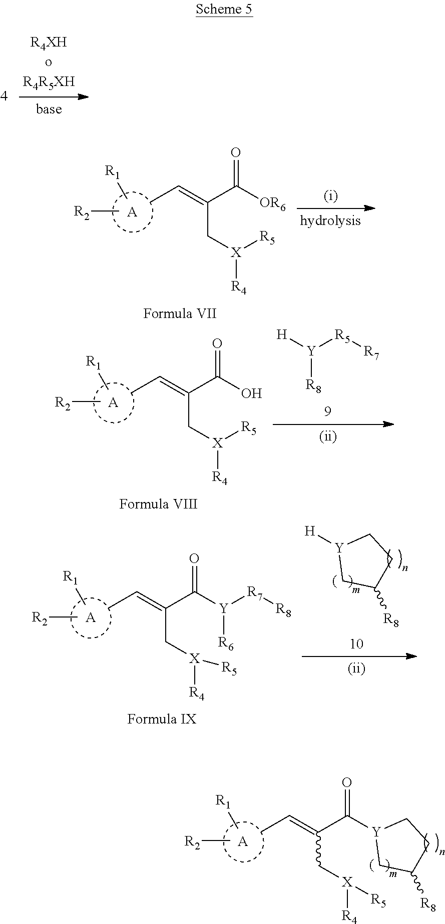

The synthesis of the compounds of formulae VIL-X is described in Scheme 5. Compound of formula 4 is reacted with a suitable nucleophile R₄XH or R₄R₅XH in a basic medium as illustrated in Scheme 5. The reaction is conducted in a solvent such as THF, dichloromethane, acetonitrile, DMF or DMSO in the presence of a base such as DABCO, DBU, Et₃N, DIPEA, potassium tert-butoxide or sodium hydride, and in an inert gas atmosphere such as nitrogen or argon. The reaction is conducted at a temperature ranging between −20 and +180° C., preferably at room temperature, to obtain the compounds having formula VII Said compounds then undergo hydrolysis in a basic medium (i) using a base such as sodium hydroxide, potassium hydroxide or lithium hydroxide in a solvent such as water, THF, 1,4-dioxane or mixtures thereof at temperatures ranging between −20 and +100° C. for a time ranging between a few minutes and 72 hours, to obtain the compounds of formula VIII. Alternatively, hydrolysis (i) can be conducted by treating a compound of formula VII with an acid such as trifluoroacetic acid, hydrochloric acid, hydrobromic acid or methanesulphonic acid in a solvent such as dichloromethane, 1,4-dioxane, water or mixtures thereof, at temperatures ranging between −20 and +100° C. for a time ranging from a few minutes to 72 hours. Compounds having formula VIII are thus obtained. Reaction (ii) described in Scheme 5 can be conducted by treating a compound of formula VIII with a suitable activating agent or coupler selected, for example, from those described in Pearson A. J. and Roush W. J. editors, “Handbook of Reagents for Organic Synthesis—Activating Agents and Protecting Groups”, John Wiley and Sons Ltd. 1999, pp. 1-482 and references cited. The preferred activating reagents are thionyl chloride, 2-(1H-benzotriazol-1-yl)-1,1,3.3-tetramethyluronium hexafluorophosphate (HBTU), 1-hydroxybenzotriazole (HOBt), 1-[bis(dimethylamino)methylene]-1H-1,2,3-triazole[4,5-b]pyridinium 3-oxide hexafluorophosphate (HATU), benzotriazol-1-yloxytripyrrolidinophosphonium hexafluorophosphate (PyBoP), carbonyldiimidazole (CDI), 1-ethyl-3-(3-dimethylaminopropyl)carbodiimide (EDC), dicyclohexylcarbodiimide (DCC) and N-hydroxysuccinimide (NHS), used alone or mixed in a suitable solvent such as dichloromethane, DMF, DMSO, acetonitrile or THF, or in mixtures thereof. The reaction can be conducted in the presence of a suitable base such as Et₃N, DIPEA or DMAP for a time ranging between a few minutes and 3 hours at temperatures ranging between −20 and +120° C. Compounds of formula 9 or 10 are added to the solution of the activated compound of formula VIII, and the mixture is left under stirring at a temperature ranging between −20 and +120° C. for a time ranging between a few minutes and 90 hours. Compounds having formulae IX and X are thus obtained.

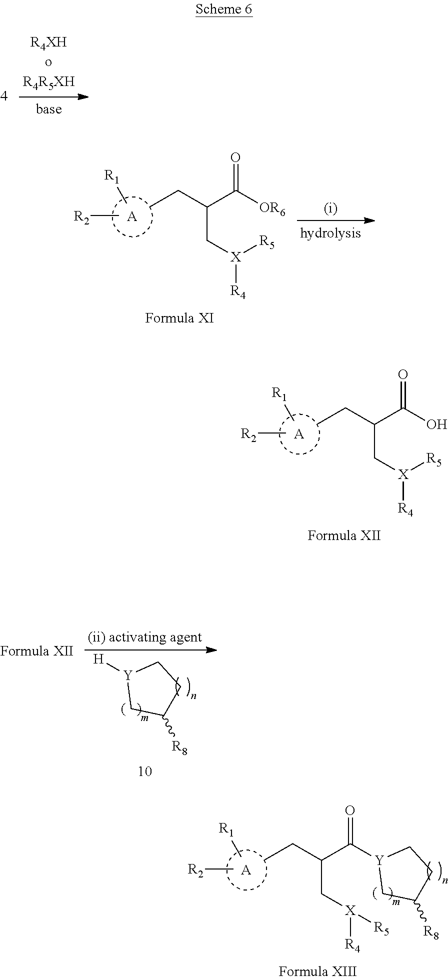

Compounds of formulae XI-XIII are synthesised as described in Scheme 6. A compound of formula 5 is reacted with a suitable nucleophile R₄XH or R₄R₅XH in a basic medium as illustrated in Scheme 6. The reaction is conducted in a solvent such as THF, dichloromethane, acetonitrile, DMF or DMSO in the presence of a base such as DABCO, DBU, Et₃N, DIPEA, potassium tert-butoxide or sodium hydride, and in an inert gas atmosphere such as nitrogen or argon. The reaction is conducted at a temperature ranging between −20 and +180° C., preferably at a temperature ranging between 20 and 40° C. The reaction provides a mixture of products which can contain various stereoisomeric forms of the desired products of formula XI which, where necessary, are separated by preparative silica gel chromatography. Compounds of formula XI then undergo hydrolysis in a basic medium using a base such as sodium hydroxide, potassium hydroxide or lithium hydroxide in a solvent such as water, THF, 1,4-dioxane or mixtures thereof at temperatures ranging between −20 and +100° C. for a time ranging between a few minutes and 72 hours, to obtain compounds of formula XII. Alternatively, hydrolysis (i) can be conducted by treating a compound of formula XI with an acid such as trifluoroacetic acid, hydrochloric acid, hydrobromic acid or methanesulphonic acid in a solvent such as dichloromethane, 1,4-dioxane, ethyl acetate, water or mixtures thereof, at temperatures ranging between −20 and +100° C. for a time ranging from a few minutes to 72 hours. Reaction (ii) described in Scheme 6 can be conducted by treating a compound of formula XIII with a suitable activating agent or coupler selected, for example, from those described in Pearson A. J. and Roush W. J. editors, “Handbook of Reagents for Organic Synthesis—Activating Agents and Protecting Groups”, John Wiley and Sons Ltd. 1999, pp. 1-482 and references cited. The preferred activating reagents are thionyl chloride, 2-(1H-benzotriazol-1-yl)-1,1,3.3-tetramethyluronium hexafluorophosphate (HBTU), 1-hydroxybenzotriazole (HOBt), 1-[bis(dimethylamino)methylene]-1H-1,2,3-triazole[4,5-b]pyridinium 3-oxide hexafluorophosphate (HATU), benzotriazol-1-yloxytripyrrolidinophosphonium hexafluorophosphate (PyBoP), carbonyldiimidazole (CDI), 1-ethyl-3-(3-dimethylaminopropyl)carbodiimide (EDC), dicyclohexylcarbodiimide (DCC) and N-hydroxysuccinimide (NHS), used alone or mixed in a suitable solvent such as dichloromethane, DMF, DMSO, acetonitrile or THF, or in mixtures thereof. The reaction can be conducted in the presence of a suitable base such as Et₃N, DIPEA or DMAP for a time ranging between a few minutes and 3 hours at temperatures ranging between −20 and +120° C. Compounds of formula 10 are added to the solution of the activated compound of formula XII, and the mixture is left under stirring at a temperature ranging between −20 and +120° C. for a time ranging between a few minutes and 90 hours. Compounds having formula XIII are thus obtained.

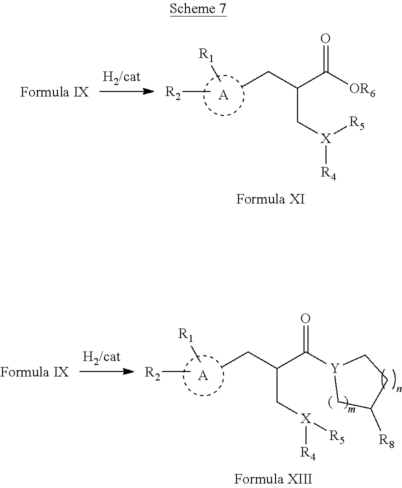

Compounds of formulae XI-XIII can also be synthesised by catalytic reduction of the compounds of formulae IX and X, as illustrated in Scheme 7. In said procedure, the reaction is conducted by dissolving the compound of formula IX or X in a solvent such a methanol, ethanol, 2-propanol, n-butanol, ethyl acetate, THF, 1,4-dioxane or mixtures thereof, and a suitable catalyst is added such as Pd supported on carbon, Pt supported on carbon, PtO₂ or, in general, the suitable substances described in Burke S. D and Danheiser R. L. eds., “Handbook of Reagents for Organic Synthesis—Oxidizing and Reducing Agents”, John Wiley and Sons Ltd 1999, pages 15-518 and references cited. The mixture is placed under vigorous stirring in H₂ gas atmosphere at a pressure ranging between 1 and 50 bars and at a temperature ranging between 0° C. and 120° C. for a time ranging between a few minutes and 72 hours. Compounds having formulae XI and XIII are thus obtained. To obtain compounds of formula XII, compounds of formula XI undergo hydrolysis according to the same procedures as described above in Scheme 6 step (i).

The examples below further illustrate the invention.

### EXAMPLES

### Synthesis Examples

### Materials and Methods

All reactions were monitored by thin-layer chromatography (TLC) on Merck 60 F254 plates (0.25 mm), which were detected with UV light and/or by spraying a solution of KMnO₄ (0.5 g in 100 mL of 0.1N NaOH) and bromocresol green (0.04 g in 100 mL of EtOH, then treated with 1N NaOH). The flash chromatography purifications used Merck silica gel with 60 mesh particles. Commercially available reagents and solvents were used without further purifications.

The ¹H and ¹³C spectra were recorded on a Jeol ECZ 600 M30, at 600 and 150 MHz respectively. The coupling constants (J) are expressed in Hertz (Hz), and the chemical shift values (δ) are supplied in ppm relative to the deuterated solvent used as internal standard.

The abbreviations used to describe plurality are: s=singlet, d=doublet, m=multiplet, dd=doublet of doublets; and the abbreviations used to identify the protons are: ArH=aromatic protons, PipH=piperidine protons.

The low resolution ESI mass spectra were recorded on a Micromass Quattro Micro TP API (Waters Corporation, Milford, MA, USA) equipped with an ESI source.

The purity of the final products was determined by reverse-phase HPLC (RP-HPLC). The tests were performed with an HP 1100 chromatography system (Agilent Technologies, Palo Alto, CA, USA) equipped with a quaternary pump (G1311A), a membrane degasser (G1379A), and a diode-array detector (DAD) (G1315B) integrated into the HP1100 system. The analysis data were processed with the HP ChemStation system (Agilent Technologies). The analysis column used was a LiChrosper 100 C18-e (250×4.6 mm, 5 μm) (Merck KGaA, 64271 Darmstadt, Germany) using the eluent indicated for each compound. All the compounds were solubilised in the mobile phase at a concentration of about 0.1 mg/mL, and injected through a 20 μL loop. The retention times (t_{R}) were obtained with a flow rate of 1.0 mL/min, and the effluent was monitored at two wavelengths (226 and 254 nm) and calibrated on the reference at 800 nm. The purity of the compounds was calculated as the percentage ratio between the main peak areas and those of any impurities at the two wavelengths, also using DAD purity analysis of the chromatographic peak. The actual purity value and the eluent used for the elution are reported for each compound at the characterisation stage. The melting points (mp) were determined in a glass capillary using a Büchi 540 melting point measuring apparatus.

Further abbreviations used are: 40-70° C. petroleum ether (PE), ethyl acetate (EtOAc), diethyl ether (Et₂O), methanol (MeOH), tetrahydrofuran (THF), dimethylformamide (DMF), dimethylsulphoxide (DMSO), retention factor (Rf), retention time (t_{R}), mass spectrometry (MS), nuclear magnetic resonance (NMR), acetic anhydride (Ac₂O), minute (min).

### Example 1—Synthesis of ethyl 2-((2-chlorophenyl)(hydroxy)methyl)acrylate (3a)

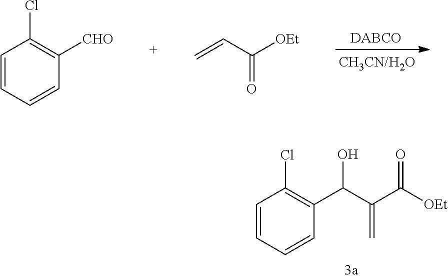

Ethyl acrylate (9.3 mL; 84.89 mmol) and water (54 mL) are added to a solution of 2-chlorobenzaldehyde (4.00 g; 28.46 mmol) in CH₃CN (9.3 mL). DABCO (3.2 g; 28.46 mmol) is then added to the mixture, and the reaction is left under stirring for 7 days at 20° C. The mixture is diluted with CH₂Cl₂ (30 mL) and extracted with 1N HCl (3×30 mL) and an NaCl saturated solution (30 mL), then dried (Na₂SO₄), and the solvent evaporated under low pressure. The residue is purified by flash chromatography on silica gel column, eluting with a PE/EtOAc 9/1 mixture. The compound is obtained as a colourless oil (5.42 g; yield 79%). CI-MS (isobutane) m/z: 241-243 [M+1]⁺; ¹H-NMR (CDCl₃): δ 7.60-7.12 (m, 4H, ArH); 6.33 (s, 1H, C═CH); 5.97 (s, 1H, CHOH); 5.60 (s, 1H, C═CH); 4.20 (q, J=7.1 Hz, 2H, CH₂CH₃); 3.44 (s, 1H, OH); 1.25 (t, 3H, CH₂CH₃); ¹³C-NMR (CDCl₃): δ 166.5; 140.9; 138.4; 132.8; 129.4; 129.0; 128.2; 127.0; 126.6; 69.2; 61.1; 14.0.

### Example 2—Synthesis of tert-butyl 2-((2-chlorophenyl)(hydroxy)-methyl)acrylate (3b)

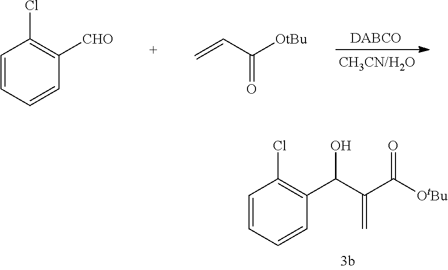

Tert-butyl acrylate (13.22 mL; 91.06 mmol), H₂O (10 mL) and DABCO (3.19 g; 28.46 mmol) are added to a solution of 2 chloro-benzaldehyde (3.20 mL; 28.46 mmol) in CH₃CN (90 mL). The reaction mixture is left under magnetic stirring at 20° C. for 7 days. The solvent is evaporated under low pressure, and the residue is taken up with CH₂Cl₂ (25 mL) and washed with 1N HCl (3×25 mL) and NaCl saturated solution (30 mL), then dried (Na₂SO₄), and the solvent evaporated under low pressure. The crude compound is purified by flash chromatography on silica gel using PE/EtOAc 9/1 as eluent. 3b is obtained as a pale yellow oil (2.9 g; yield 38%). MS (ESI) m/z: 269-271 [M+H]⁺; ¹H-NMR (CDCl₃): δ 7.51 (dd, J=7.7. 1.6 Hz, 1H, ArH); 7.35 (dd, J=7.9, 1.2 Hz, 1H, ArH); 7.28 (d, J=7.6, 1.1 Hz, 1H, ArH); 7.25-7.23 (d, 1H, ArH); 6.25 (s, 1H, C═CH); 5.93 (s, 1H, OH—CH); 5.53 (s, 1H, C═CH); 1.43 (s, 9H, CH₃); 1.25 (s, 1H, OH).

### Example 3—Synthesis of ethyl 2-(acetoxy(2-chlorophenyl)methyl)acrylate (4a)

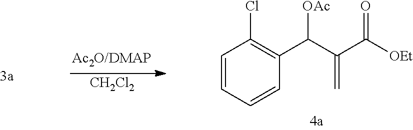 [SMILES: "CH2Cl2" ; C=C(C(=O)OCC)C(OC(C)=O)c1ccccc1Cl]

Acetic anhydride (0.509 g; 4.99 mmol) dissolved in CH₂Cl₂ (10 mL) is added 5 slowly over a period of 1 hour to a solution of 3a (1.00 g; 4.16 mmol) and DMAP (102 mg, 0.831 mmol) in CH₂Cl₂ (10 mL) at 0° C., maintaining the mixture under stirring at 20° C. The reaction mixture is extracted with water (15 mL) and NaHCO₃ 10% w/v (3×30 mL), then with a NaCl saturated solution (30 mL). The organic phase is dried (Na₂SO₄) and the solvent is evaporated under low pressure. The residue is purified by flash chromatography on silica gel using a PE/EtOAc 9/1 mixture as eluent. Compound 4a is obtained as a colourless oil (0.783 g; yield 67%). CI-MS (isobutane) m/z: 283-285 [M+1]⁺; ¹H-NMR (CDCl₃): δ, 7.47-7.26 (m, 4H, ArH); 7.06 (s, 1H, CH); 6.47 (s, 1H, C═CH); 5.63 (s, 1H, C═CH); 4.19 (q, J=7.1 Hz, 2H, CH₂CH₃); 2.12 (s, 3H, CH₃); 1.23 (t, J=7.1 Hz, 3H, CH₂CH₃).

### Example 4—Synthesis of ethyl 2-(2-chlorobenzyl)acrylate (5a) (PROCEDURE A)

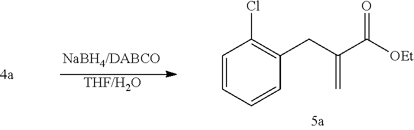

NaBH₄ (0.129 g; 3.42 mmol) and DABCO (0.384 g; 3.42 mmol) are added in succession to a solution of 4a (0.968 g; 3.42 mmol) in THF/H₂O 1/1 (40 mL), maintained under an inert atmosphere (N₂). The reaction is left under stirring for 1 hour. The mixture is diluted with water (20 mL), extracted with EtOAc (3×60 mL) and dried (Na₂SO₄), and the solvent is evaporated under low pressure. The residue is purified by flash chromatography on silica gel using a PE/EtOAc 95/5 mixture as eluent; compound 5a is obtained as a colourless oil (0.649 g; yield 84%). CI-MS (isobutane) m/z: 225-227 [M+1]⁺; ¹H-NMR (CDCl₃): δ, 7.55-6.99 (m, 4H, ArH); 6.27 (d, 1H, C═CH); 5.33 (d, 2H, C═CH); 4.22 (q, J=7.1 Hz, 2H, CH₂CH₃); 3.76 (s, 2H, Ph-CH₂); 1.29 (t, J=7.1 Hz, 3H, CH₂CH₃).

### Example 5—Synthesis of ethyl 2-(2-chlorobenzyl)acrylate (5a) (Procedure B)

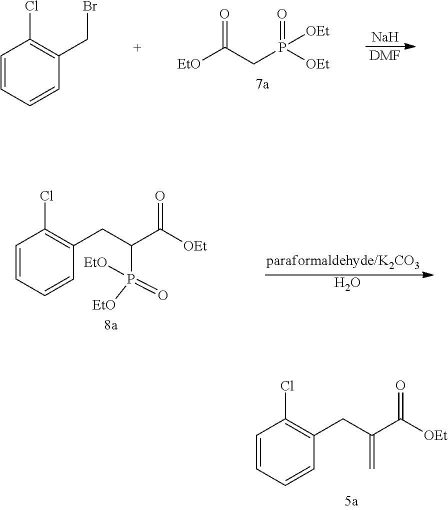

60% NaH in mineral oil (0.820 g; 20.5 mmol) is added to a solution of ethyl diethoxyphosphorylacetate (3.93 g; 17.6 mmol) in anhydrous DMF (30 mL) placed at 0° C. in an inert atmosphere (N₂). 2-chlorobenzyl bromide (3.00 g; 14.6 mmol) is added after 2 hours, and the reaction is left under stirring in an inert atmosphere (N₂) for 16 hours. H₂O (15 mL) is added to the reaction, and the mixture is extracted with EtOAc (2×15 mL), washed with a NaCl saturated solution (15 mL) and dried (Na₂SO₄), and the solvent is removed under low pressure. The residue is purified by flash chromatography on silica gel using a PE/EtOAc 7/3 mixture to elute the unreacted 2-chlorobenzyl bromide, and then using PE/EtOAc 1/1 to obtain 3.42 g of compound 5a (yield 67%). Intermediate 8a is not further characterised, but is used directly in the next step.

A solution of K₂CO₃ (4.00 g; 29.0 mmol) in H₂O (60 mL) is added to a solution of intermediate 8a (3.36 g; 9.66 mmol) and paraformaldehyde (1.91 g; 0.064 mol) in H₂O (60 mL), and the reaction is left under stirring for 16 hours at 90° C. The mixture is extracted with EtOAc (2×20 mL), washed with a NaCl saturated solution (20 mL) and dried (Na₂SO₄), and the solvent is removed under low pressure. The residue is purified by flash chromatography on silica gel using a PE/EtOAc 7/3 mixture to obtain 1.63 g of compound 5a (yield 75%). The characterisation of this compound is identical to that of the compound obtained by Procedure A.

### Example 6—Synthesis of tert-butyl 2-(2-chlorobenzyl) acrylate (5b)

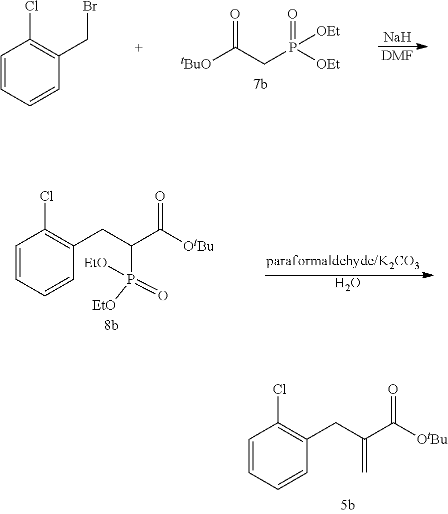

60% NaH in mineral oil (1.23 g; 30.7 mmol) is added to a solution of tert-butyldiethylphosphonoacetate (6.17 mL; 26.3 mmol) in anhydrous DMF (40 mL) maintained at 0° C. under an inert atmosphere (N₂). The reaction mixture is stirred for 2.5 hours at 25° C.; 2-chlorobenzyl bromide (2.84 mL; 21.9 mmol) is then added drop by drop at 0° C., and the solution is left under magnetic stirring for 2 hours at 25° C. The reaction mixture is cooled to 0° C., and water (20 mL) is added. After 16 hours the solvent is evaporated under low pressure. The crude compound is solubilised in diethyl ether (30 mL) and washed with H₂O (2×10 mL) and an NaCl saturated solution (10 mL), then dried (Na₂SO₄) and concentrated under low pressure to give tert-butyl 3-(2-chlorophenyl)-2-(diethyloxyphosphoryl)propanoate (8b) (8.20 g; yield 99%) as a white solid, which is used in the next step without further purification.

A solution of K₂CO₃ (8.62 g; 62.4 mmol) in H₂O (60 mL) is added to a solution of 8b (8.20 g; 21.9 mmol) and paraformaldehyde (5.25 mL; 175 mmol) in H₂O (80 mL). The reaction mixture is heated at 90° C. for 16 hours. The mixture is cooled to room temperature and extracted with EtOAc (3×40 mL). The organic phase is washed with a NaCl saturated solution (15 mL) and dried (Na₂SO₄), and the solvent is evaporated under low pressure. The crude compound is purified by flash chromatography on silica gel using PE/EtOAc 95/5 as eluent. Compound 5b is obtained (4.98 g; yield 90%) as a colourless oil. MS (ESI) m/z: 275-277 [M+Na]⁺; ¹H-NMR (CDCl₃): δ, 7.36-7.15 (in, 4H, ArH); 6.17 (s, 1H, C═CHH); 5.25 (m, 1H, C═CHH); 3.71 (s, 2H, CH₂); 1.45 (s, 9H, CH₃). ¹³C-NMR (CDCl₃): δ, 166.1; 139.9; 136.9; 134.5; 130.9; 129.6; 127.8; 126.8; 126.0; 80.9; 35.5; 28.0.

### Example 7—Synthesis of the Compounds of Formula INF38 (General Procedure)

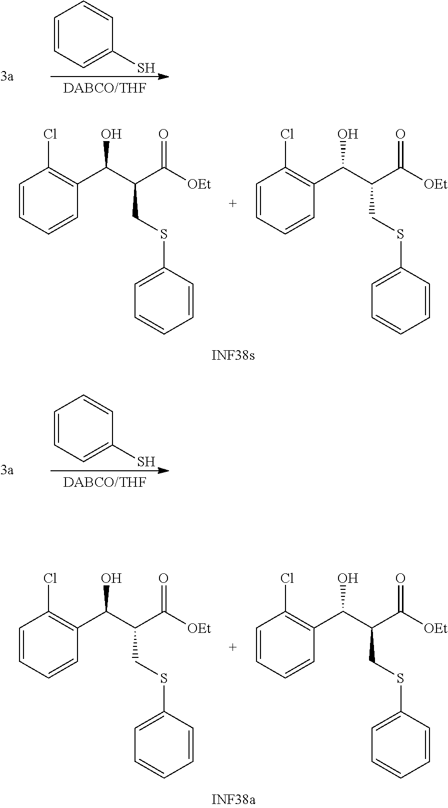 [SMILES: Sc1ccccc1 ; CCOC(=O)[C@@H](CSc1ccccc1)[C@H](O)c1ccccc1Cl ; CCOC(=O)[C@H](CSc1ccccc1)[C@@H](O)c1ccccc1Cl ; Sc1ccccc1 ; CCOC(=O)[C@H](CSc1ccccc1)[C@H](O)c1ccccc1Cl ; CCOC(=O)[C@@H](CSc1ccccc1)[C@@H](O)c1ccccc1Cl]

DABCO (0.190 g; 1.69 mmol) and thiophenol (0.103 mL; 0.997 mmol) are added to a solution of 3a (0.200 g; 0.831 mmol) in distilled THF (15 mL), maintained under an inert atmosphere (N₂), and the reaction is placed under stirring at 20° C. for 2.5 hours. The mixture is extracted with CH₂Cl₂ (3×15 mL), 1N HCl (25 mL) and H₂O (25 mL). The organic phase is dried (Na₂SO₄) and the solvent is evaporated under low pressure. The crude compound is purified by flash chromatography on silica gel, eluting with PE/EtOAc 95/5 then with PE/EtOAc 9/1. The syn diastereomer INF38s (65%) and the anti diastereomer INF38a (11%) are isolated in this way.

### Ethyl(2S,3S)-3-(2-chlorophenyl)-3-hydroxy-2-(phenylsulphanylmethyl)-propanoate and ethyl(2R,3R)-3-(2-chlorophenyl)-3-hydroxy-2-(phenylsulphanylmethyl)propanoate (INF38s)

CI-MS (isobutane) m/z: 351-353 [M+1]⁺; ¹H-NMR (CDCl₃): δ, 7.37-6.96 (m, 911, ArH); 5.42 (t, J=3.0 Hz, 1H, CHOH); 4.12 (q, J=7.2 Hz, 2H, CH₂CH₃); 3.38-2.92 (m, 3H aliphatic and OH); 1.19 (t, J=7.1 Hz, 3H, CH₂CH₃). ¹³C-NMR (CDCl₃): δ, 173.6; 137.7; 135.5; 131.7; 129.6; 129.02; 128.98; 128.8; 128.2; 126.9; 126.0; 70.6; 61.3; 49.3; 29.8; 14.0. Cytotoxicity (MTT assay): IC₅₀>100 μM.

### Ethyl(2S,3S)-3-(2-chlorophenyl)-3-hydroxy-2-(phenylsulphanylmethyl)-propanoate and ethyl(2S,3R)-3-(2-chlorophenyl)-3-hydroxy-2-(phenylsulphanylmethyl)propanoate (INF38a)

CI-MS (isobutane) m/z 351-353 [M+1]⁺; ¹H-NMR (CDCl₃): δ, 7.44-7.07 (m, 9H, ArH); 5.42-5.27 (dd, J=7.8 Hz, 1H, CHOH), 4.09 (q, J=7.1 Hz, 2H, CH₂CH₃); 3.58 (d, J=7.9 Hz, 1H, OH); 3.39-3.25 (m, 1H, CHCH₂); 3.22-3.07 (m, 2H, CH₂S); 1.06 (t, J=7.1 Hz, 3H, CH₂CH₃). ¹³C-NMR (CDCl₃): 5, 173.4; 138.9; 135.1; 131.9; 129.9; 129.5; 129.0; 127.3; 127.0; 126.6; 70.8; 61.1; 50.2; 33.5; 14.0. Cytotoxicity (MTT assay): IC₅₀>100 μM.

### Example 8—Synthesis of ethyl 3-(2-chlorophenyl)-3-hydroxy-2-((phenylsulphinyl)methyl)propanoate (INF44)

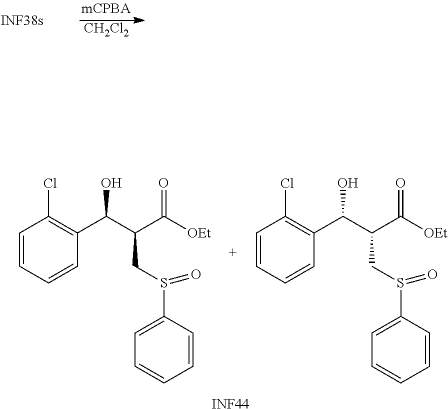

75% mCPBA (0.085 g; 0.371 mmol) is added to a solution of INF38s (0.130 g; 0.371 mmol) in CH₂Cl₂ (10 mL). The reaction mixture is left under magnetic stirring at 20° C. for 18 hours. The reaction mixture is extracted with a 10% w/v solution of NaOH (3×20 mL) and NaCl saturated solution (20 mL), the organic phase is dried (Na₂SO₄), and the solvent is removed under low pressure. The crude compound is purified by flash chromatography on silica gel column using a PE/EtOAc 7/3 mixture as eluent. Compound INF44 is obtained as a colourless oil (0.092 g; yield 68%). CI-MS (isobutane) m/z: 367-369 [M+1]⁺; ¹H-NMR (CDCl₃): δ, 7.54-7.11 (m, 9H, ArH); 5.49 (d, J=2.8 Hz, 1H, CHOH); 4.19 (q, J=7.1 Hz, 2H, CH₂CH₃); 4.14-4.00 (m, 1H, CHCOOEt); 3.78 (s, 1H, OH); 3.41-3.16 (m, 2H, CH₂SO); 1.18 (t, J=7.0 Hz, 3H, CH₂CH₃). Cytotoxicity (MTT assay): IC₅₀>100 μM.

### Example 9—Synthesis of ethyl 3-(2-chlorophenyl)-3-hydroxy-2-((phenylsulphonyl)methyl)propanoate (INF45)

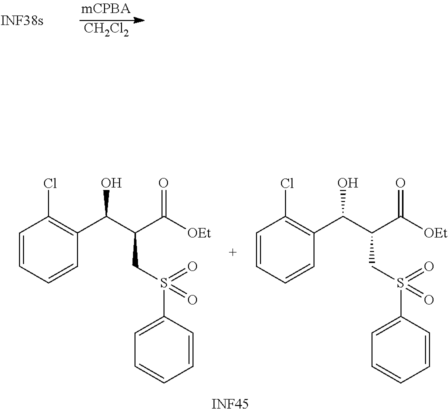

75% mCPBA acid (0.211 g; 0.918 mmol) is added to a solution of INF38s (0.103 g; 0.306 mmol) in CH₂Cl₂ (10 mL). The reaction mixture is left under magnetic stirring at 20° C. for 18 hours. The reaction mixture is extracted with a 10% w/v solution of NaOH (3×20 mL) and with NaCl saturated solution (20 mL). The organic phase is dried (Na₂SO₄), and the solvent is removed under low pressure. The crude compound is purified by flash chromatography on silica gel column using a PE/EtOAc 7/3 mixture as eluent. Compound INF45 is obtained as a white solid (0.098 g; yield: 82%). CI-MS (isobutane) m/z: 383-385 [M+1]⁺; ¹H-NMR (CDCl₃): δ, 7.70-7.02 (m, 9H, ArH); 5.30 (d, J=3.8 Hz, 1H, CHOH); 4.03 (q, J=7.1 Hz, 2H, CH₂CH₃); 3.75 (dt, J=10.2; 4.2 Hz, 1H, CHCOOEt); 3.32-3.06 (m, 2H, CH₂S02); 1.17 (t, 3H, J=7.1 Hz, CH₂CH₃); ¹³C-NMR (CDCl₃): δ, 172.0; 138.6; 137.32; 137.29; 134.1; 134.0; 131.9; 130.5; 130.24; 130.20; 129.8; 129.6; 128.6, 128.3; 127.4; 70.9; 62.2; 52.7; 45.2; 45.2; 14.3. Mp: 78.8-83.1° C. Cytotoxicity (MTT assay): IC₅₀>100 μM.

### Example 10—Synthesis of ethyl (Z)-3-(2-chlorophenyl)-2-((phenylthio)methyl)acrylate (INF42)

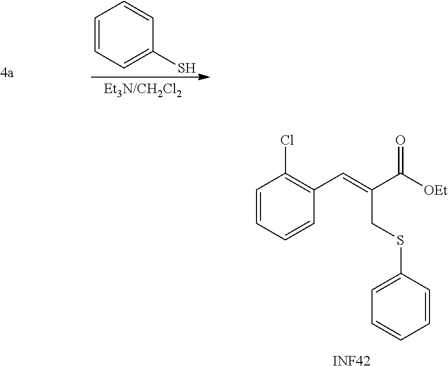 [SMILES: Sc1ccccc1 ; CCOC(=O)/C(=C/c1ccccc1Cl)CSc1ccccc1 ; "INF42"]

Thiophenol (0.240 mL; 2.33 mmol) and triethylamine (0.355 mL; 2.55 mmol) are added to a solution of 4a (0.60 g; 2.12 mmol) in CH₂Cl₂ (7.5 mL) maintained in an inert atmosphere (N₂). The reaction is left under vigorous stirring for 30 minutes at 20° C. The reaction mixture is then diluted with H₂O (5 mL) and extracted with 1N HCl (3×20 mL) and NaCl saturated solution (20 mL). The organic phase is dried (Na₂SO₄), and the solvent is removed under low pressure. The residue is purified by flash chromatography on silica gel column, eluting with pure PE and then with a PE/EtOAc 95/5 mixture. Two fractions are obtained, the first containing the mixture of the two isomers (E/Z) and the second containing the pure Z isomer (INF42; 0,260 g; yield: 37%). CI-MS (isobutane) m/z: 333-335 [M+1]⁺; ¹H-NMR (CDCl₃): δ, 7.80 (s, 1H, C═CH); 7.34-7.14 (m, 9H, ArH); 4.29 (q, J=7.1 Hz, 2H, CH₂CH₃); 3.91 (s, 2H, CH₂S); 1.33 (t, J=7.1 Hz, 3H, CH₂CH₃). Cytotoxicity (MTT assay): IC₅₀>100 μM.

### Example 11—Synthesis of (Z)-ethyl 3-(2-chlorophenyl)-2-((phenylsulphinyl)methyl)acrylate (INF50)

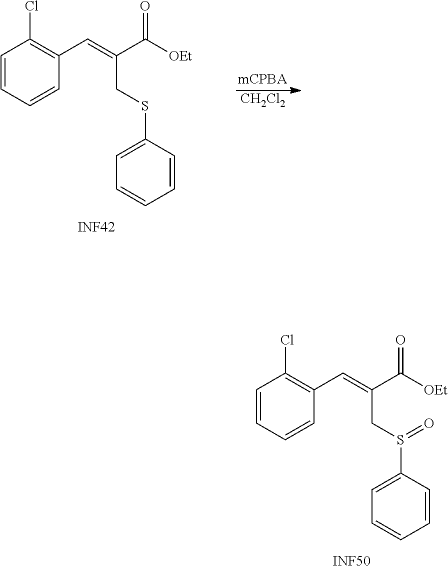 [SMILES: CCOC(=O)/C(=C/c1ccccc1Cl)CSc1ccccc1 ; "INF42" ; "CH2Cl2" ; CCOC(=O)/C(=C/c1ccccc1Cl)CS(=O)c1ccccc1 ; "INF50"]

75% mCPBA (0.105 g; 0.449 mmol) is added to a solution of INF42 (0.150 g; 0.449 mmol) in CH₂Cl₂ (10 mL), and the reaction mixture is left under magnetic stirring at 20° C. for 18 hours. The reaction mixture is extracted with a 10% w/v solution of NaOH (3×20 mL) and NaCl saturated solution (20 mL), then dried (Na₂SO₄), and the solvent is removed under low pressure. The crude compound is purified by flash chromatography on silica gel column using a PE/EtOAc 9/1 mixture as eluent. Compound INF50 is obtained as a colourless oil (0.156 g; yield: 88%). CI-MS (isobutane) m/z: 349-351 [M+1]⁺; ¹H-NMR (CDCl₃): δ, 8.08 (s, 1H, C═CH); 7.82-7.18 (m, 9H, ArH; 4.36-4.11 (m, 2H, CH₂CH₃); 3.91 (m, 2H, CH₂SO); 1.30 (t, J=7.1 Hz, 3H, CH₂CHs); ¹³C-NMR (CDCl₃): δ, 166.6; 144.2; 143.4; 134.4; 132.9; 131.7; 131.3; 130.9; 129.9; 129.6; 127.3; 124.8; 124.5; 62.1; 57.3; 14.6. Cytotoxicity (MTT assay): IC₅₀ 9.7±0.2 μM.

### Example 12—Synthesis of (Z)-ethyl 3-(2-chlorophenyl)-2-((cyclohexylthio)methyl)acrylate (INF56)

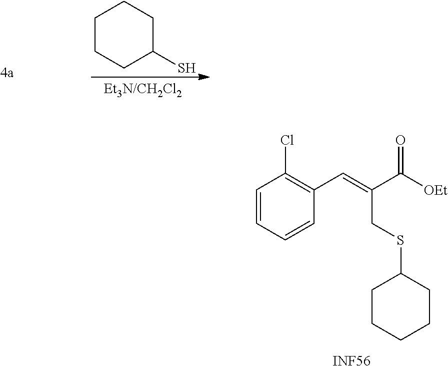 [SMILES: SC1CCCCC1 ; CCOC(=O)/C(=C/c1ccccc1Cl)CSC1CCCCC1 ; "INF56"]

Triethylamine (0.037 mL; 0.265 mmol) and cyclohexylmercaptan (0.027 mL; 0.230 mmol) are added to a solution of 4a (0.050 g; 0.177 mmol) in DMF (5 mL), and the mixture is left under vigorous stirring at 60° C. under an inert atmosphere (N₂) for 18 hours. The reaction is treated with H₂O (30 mL) and EtOAc (50 mL), the phases are separated, and the organic phase is further washed with H₂O (3×60 mL). The organic phase is dried (Na₂SO₄) and the solvent is evaporated under low pressure. The residue is purified by flash chromatography on silica gel column using a PE/EtOAc 98/2 mixture as eluent. The compound INF56 (0.0234 g; yield 39%) is obtained as a pale yellow oil which solidifies with time. CI-MS (isobutane) m/z: 339-341 [M+1]⁺; ¹H-NMR (CDCl₃): δ, 7.76 (s, 1H, C═CH); 7.61-7.14 (m, 4H, ArH); 4.32 (q, J=7.0 Hz, 2H, CH₂CH₃); 3.53 (s, 2H, CH₂S); 1.86-1.47 (m, 5H, H cyclohexyl); 1.37 (t, J=7.1 Hz, 3H, CH₂CH₃); ¹³C-NMR (CDCl₃): δ, 167.4; 136.9; 134.6; 134.2; 132.4; 130.9; 130.2; 130.0; 127.1; 61.7; 44.2; 33.8; 26.9; 26.5; 26.2; 14.7. Mp: 49.4-53.2° C. Cytotoxicity (MTT assay): IC₅₀>100 μM.

### Example 13—Synthesis of (E)-ethyl 3-(2-chlorophenyl)-2-(phenoxymethyl)acrylate (57)

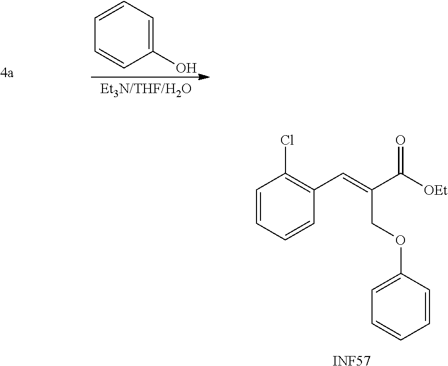 [SMILES: Oc1ccccc1 ; CCOC(=O)/C(=C/c1ccccc1Cl)COc1ccccc1 ; "INF57"]

K₂CO₃ (0.137 g; 0.991 mmol) and phenol (0.080 g; 0.851 mmol) are added to a solution of 5a (0.200 g; 0.707 mmol) in THF/H₂O 1/3 (20 mL). The reaction mixture is left under vigorous stirring at 80° C. for 18 hours. After 18 hours, a 2% w/v solution of NaOH (10 mL) is added and the mixture is extracted with EtOAc (3×25 mL), dried (Na₂SO₄), and the solvent removed under low pressure. The crude compound is purified by flash chromatography on silica gel column using a PE/EtOAc 9/1 mixture as eluent. INF57 is thus obtained as a white amorphous semisolid (0.104 g; yield: 48%). CI-MS (isobutane) m/z: 316-318 [M+1]⁺; ¹H-NMR (CDCl₃): δ, 7.88 (s, 1H, C═CH); 7.44-7.02 (m, 9H, ArH); 4.61 (s, 2H, CH₂O); 4.32 (q, J=7.1 Hz, 2H, CH₂CH₃); 1.37 (t, J=7.1 Hz, 3H, CH₂CH₃). Cytotoxicity (MTT assay): IC₅₀>100 μM.

### Example 14—Synthesis of ethyl 2-(2-chlorobenzyl)-3-(phenylthio)propanoate (INF43)

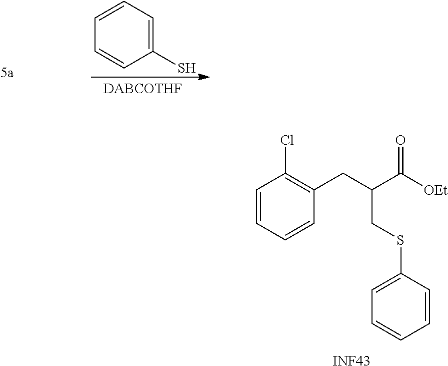 [SMILES: Sc1ccccc1 ; CCOC(=O)C(CSc1ccccc1)Cc1ccccc1Cl ; "INF43"]

DABCO (0.326 g; 2.91 mmol) and thiophenol (0.358 mL; 3.49 mmol) are added to a solution of 5a (0.350 g; 1.45 mmol) in THF (15 mL) in an inert atmosphere (N₂), and the reaction is left under magnetic stirring for 4 hours. The mixture is diluted with CH₂Cl₂ (15 mL) and extracted with 1N HCl (3×30 mL) and NaCl saturated solution (30 mL), dried (Na₂SO₄), and the solvent removed under low pressure. The residue is purified by flash chromatography on silica gel column using a PE/EtOAc 98/2 mixture as eluent. Compound INF43 is obtained as a colourless oil (0.247 g; yield: 50%). CI-MS (isobutane) m/z: 335-337 [M+1]⁺; ¹H-NMR (CDCl₃): δ, 7.44-7.07 (m, 9H, ArH); 4.03 (q, J=7.1 Hz, 2H, CH₂CH₃); 3.37-2.92 (in, 5H, H aliphatic); 1.12 (t, J=7.1 Hz, 3H, CH₂CH₃). ¹³C-NMR (CDCl₃): δ, 174.0; 136.6; 136.0; 134.6; 131.7; 130.2; 130.0; 129.4; 128.6; 127.2; 126.8; 61.1; 45.9; 36.1, 35.9; 14.5. Cytotoxicity (MTT assay): IC₅₀>100 μM.

### Example 15—Synthesis of ethyl 2-(2-chlorobenzyl)-3-(phenylsulphinyl)propanoate (INF48)

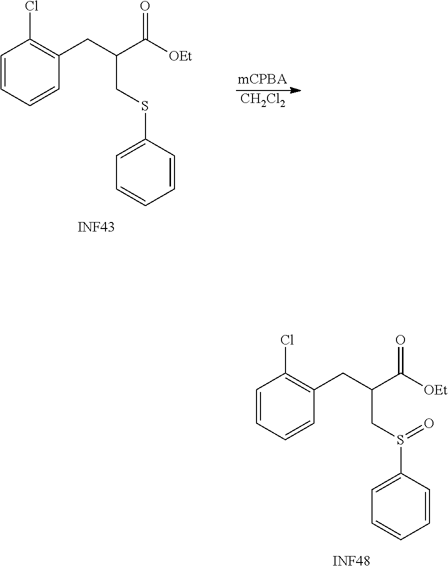 [SMILES: CCOC(=O)C(CSc1ccccc1)Cc1ccccc1Cl ; "INF43" ; "CH2Cl2" ; CCOC(=O)C(Cc1ccccc1Cl)CS(=O)c1ccccc1 ; "INF48"]

75% mCPBA (0.067 g; 0.300 mmol) is added to a solution of INF43 (0.100 g; 0.300 mmol) in CH₂Cl₂ (10 mL), and the reaction mixture is left under magnetic stirring at 20° C. for 18 hours. The solution is extracted with 1% w/v NaOH (3×20 mL) and NaCl saturated solution (20 mL), dried (Na₂SO₄), and the solvent evaporated under low pressure. The crude compound is purified by flash chromatography on silica gel column using a PE/EtOAc 8/2 mixture as eluent. Compound INF48 is obtained as a colourless oil (0.087 g; yield: 83%). CI-MS (isobutane) m/z: 351-353 [M+1]⁺; ¹H-NMR (CDCl₃): δ, 7.66-7.12 (m, 9H, ArH); 4.29 (q, J=7.1 Hz, 2H, CH₂CH₃); 3.34-2.81 (m, 5H, H aliph); 1.33 (t, J=7.1 Hz, 31H, CH₂CH₃). ¹³C-NMR (CDCl₃): δ, 173.2; 144.2; 143.6; 134.6; 131.4; 130.1; 130.1; 129.7; 129.6; 128.8; 127.3; 124.6; 124.3; 61.6; 58.6; 40.3; 36.1; 14.4. Cytotoxicity (MTT assay): IC₅₀>35.1±10.1 μM.

### Example 16—Synthesis of ethyl 2-(2-chlorobenzyl)-3-(phenylsulphonyl)-propanoate (INF49)

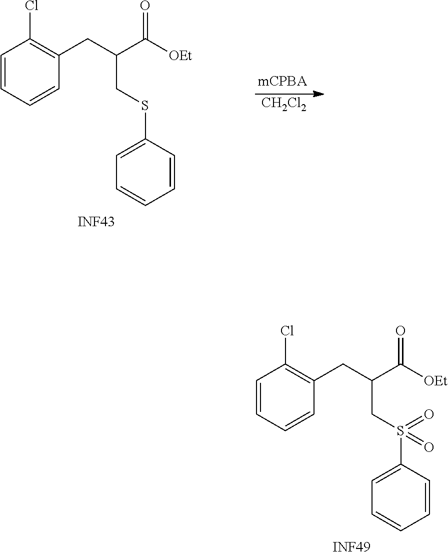 [SMILES: CCOC(=O)C(CSc1ccccc1)Cc1ccccc1Cl ; "INF43" ; "CH2Cl2" ; CCOC(=O)C(Cc1ccccc1Cl)CS(=O)(=O)c1ccccc1 ; "INF49"]

75% mCPBA (0.207 g; 0.898 mmol) is added to a solution of INF43 (0.100 g; 0.300 mmol) in CH₂Cl₂ (10 mL), and the reaction is left under magnetic stirring at 20° C. for 18 hours. The solution is diluted with water (10 mL) and extracted with a 10% w/v solution of NaHCO₃ (3×20 mL) and NaCl saturated solution (20 mL), dried (Na₂SO₄) and the solvent removed under low pressure. The crude compound is purified by flash chromatography on silica gel column using a PE/EtOAc 8/2 mixture as eluent. Compound INF49 is obtained as a colourless oil (0.078 g; yield: 72%). CI-MS (isobutane) m/z: 367-369 [M+1]⁺; ¹H-NMR (CDCl₃): δ, 7.95-6.98 (m, 9H, ArH); 3.94 (q, J=7.1 Hz, 2H, CH₂CH₃); 3.75 (dd, J=14.2; 9.8 Hz, 1H, Ph-CH); 3.27 (ddd, J=17.7; 7.9; 2.7 Hz, 1H, CHCOOEt); 3.14 (dd, J=14.3; 2.8 Hz, 1H, Ph-CH); 3.08-2.92 (m, 2H, CH₂SO₂); 1.09 (t, J=7.1 Hz, 3H, CH₂CH₃); ¹³C-NMR (CDCl₃): δ, 172.3; 138.7; 134.5; 134.2; 133.8; 131.1; 129.8; 129.2; 128.6; 128.2; 126.9: 61.3; 56.7; 40.2; 36.1; 13.9. Cytotoxicity (MTT assay): IC₅₀>100 μM.

### Example 17—Synthesis of ethyl 3-((2-((tert-butoxycarbonyl)amino)-3-methoxy-3-oxopropyl)thio)-2-(2-chlorobenzyl)propanoate (INF55)

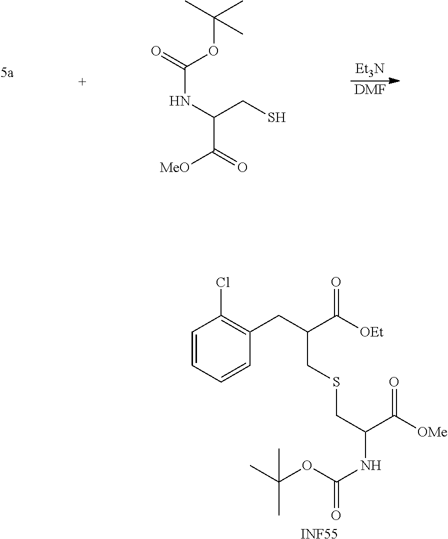

5a (0.053 g; 0.236 mmol) is added to a solution of methyl (tert-butoxycarbonyl)cysteinate (0.072 g; 0.07 mmol) and triethylamine (0.0427 mL; 0.307 mmol) in DMF (3 mL), maintained under an inert atmosphere (N₂). The reaction is placed at 60° C. for 18 hours. The reaction mixture is diluted with 0.1N HCl (10 mL), then extracted with EtOAc (3×10 mL), dried (Na₂SO₄), and the solvent removed under low pressure. The crude compound is purified by flash chromatography on silica gel column using a PE/EtOAc 9/1 mixture as eluent; compound INF55 is thus obtained (0.0454 g; yield: 42%). CI-MS (isobutane) m/z: 459-461 [M+1]⁺; ¹H-NMR (DMSO): δ, 7.44-7.06 (m, 4H, ArH); 5.38 (s, 1H, NH); 4.52 (s, 1H, CHNH); 4.08 (q, J=7.0 Hz, 2H, CH₂CH₃); 3.74 (s, 311, COOCH₃); 3.16-2.58 (m, 7H, H aliph); 1.45 (s, 9H, C(CH₃)₃); 1.14 (t, J=7.1 Hz, 3H, CH₂CH₃); ¹³C-NMR (CDCl₃): δ, 174.0; 171.8; 136.5; 134.6; 131.6; 130.0; 128.6; 127.1; 77.9; 77.4; 77.0; 61.2; 53.6; 53.0; 46.2; 46.0; 36.0; 35.6; 34.8; 28.6; 14.5. Cytotoxicity (MTT assay): IC₅₀>100 μM.

### Example 18—Synthesis of (S,Z)-2-acetamido-4-((3-(2-chlorophenyl)-2-(ethoxycarbonyl)allyl)thio)butanoic acid (INF85)

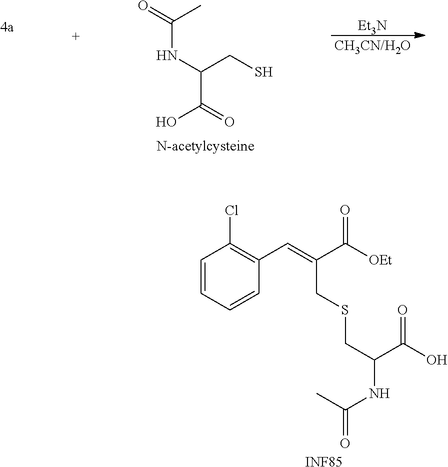

Triethylamine (0.190 mL; 1.85 mmol) and N-acetyleysteine (0.302 mg; 1.85 mmol) are added to a solution of 4a (0.209 g; 0.740 mmol) in CH₃CN/H₂O 2/1 (6 mL), maintained under an inert atmosphere (N₂), and the reaction mixture is left under stirring at 20° C. for 16 hours. 0.1N NaOH (10 mL) is added to the reaction mixture, which is extracted with EtOAc (25 mL). The aqueous phase is acidified with 2N HCl and extracted with EtOAc (3×30 mL). The organic phases are washed with a NaCl saturated solution (25 mL), dried (Na₂SO₄), and evaporated under low pressure. The crude compound is purified by flash chromatography on silica gel column, eluting with CH₂Cl₂/EtOAc 1/1 (+0.1% HCOOH) to provide INF85 as a colourless oil (0.099 g; yield: 35%). Negative ESI/MS m/z: 384-386 [M−H]⁻; ¹H NMR (CDCl₃): δ, 8.38 (br, 1H, COOH); 7.83 (s, 1H, C═CH); 7.57-7.20 (m, 4H, ArH); 7.02 (d, J=7.3 Hz, NH); 4.99-4.67 (m, 1H, CH); 4.33 (q, J=7.1 Hz, CH₂CH₃); 3.57 (q, J=12.1 Hz, CH₂S); 3.17-2.87 (m, 2H, CHCH₂S); 2.07 (s, 3H, CH₃); 1.37 (t, J=7.1 Hz, 3H, CH₃); ¹³C NMR (CDCl₃): δ 173.3; 172.5; 167.5, 138.7; 134.5; 133.5; 130.9; 130.6; 130.1; 127.4; 62.1; 52.5; 34.8; 30.1; 26.6; 22.7; 14.2. Cytotoxicity (MTT assay): IC₅₀>100 μM.

### Example 19—Synthesis of (Z)-3-(2-chlorophenyl)-2-((phenylthio)methyl)acrylic acid (INF80)

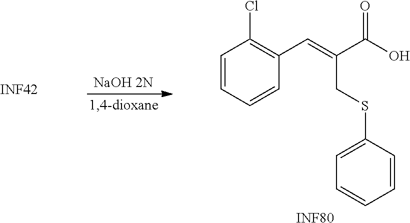 [SMILES: "INF42" ; O=C(O)/C(=C/c1ccccc1Cl)CSc1ccccc1 ; "INF80"]

Compound INF42 (0.828 g; 2.49 mmol) is dissolved in 1,4-dioxane (10 mL), and 2N NaOH (10 mL) is added. The reaction mixture is placed under stirring at 20° C. for 16 hours, and then acidified to pH=1 with 2N HCl (10 mL) and extracted with EtOAc (3×50 mL). The organic phases are washed with a NaCl saturated solution, dried (Na₂SO₄), and evaporated under low pressure to obtain compound INF80 as a cream-coloured solid (0.759 g; yield: 88%). Mp: 97.6-99.9° C.; Negative ESI/MS m/z: 303-305 [M−H]⁻; ¹H NMR (CDCl₃): δ, 12.36 (br, 1H, COOH); 7.98 (s, 1H, C═CH); 7.39-7.20 (m, 9H, ArH); 3.93 (s, 2H, CH₂S); ¹³C NMR (CDCl₃): δ 172.6; 140.2; 135.3; 134.4; 133.0; 131.2; 130.3; 130.1; 129.7; 129.6; 128.9; 127.0; 126.7; 32.0. Cytotoxicity (MTT assay): IC₅₀ 92.8±1.6 μM.

### Example 20—Synthesis of (Z)-3-(2-chlorophenyl)-2-((phenylthio)methyl)-N-propylacrylamide (INF82)

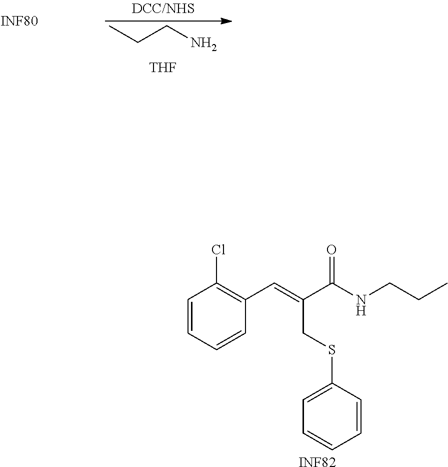

Compound INF80 (0.222 g; 0.731 mmol) is dissolved in THF (5 mL), and DCC (0.151 g; 0.731 mmol) and NHS (0.0841 g; 0.731 mmol) are added to the resulting solution, maintained at 0° C. The mixture is placed under magnetic stirring at 0° C. for 10 minutes, and then at 20° C. for 2 hours. Propylamine (0.120 mL; 1.426 mmol) is then added to the reaction mixture, and the reaction is stirred at 20° C. for 16 hours. The resulting suspension is filtered, the filtrate diluted with 2N HCl (10 mL), and extracted with EtOAc (3×25 mL). The combined organic phases are washed with a NaCl saturated solution, dried (Na₂SO₄), and evaporated under low pressure. The crude compound is purified by flash chromatography on silica gel column, using CH₂Cl₂/EtOAc 98/2 as eluent to provide INF82 as a colourless oil (0.131 g; yield: 52%). Positive ESI/MS m/z: 346-348 [M+H]⁺; ¹H-NMR (CDCl₃): δ, 7.47 (s, 1H, C═CH); 7.39-7.26 (m, 9H, ArH); 6.42 (s, 1H, NH); 3.93 (s, 2H, CH₂S); 3.35 (q, J=6.5 Hz, 2H, CH₂); 1.70-1.52 (m, 2H, CH₂); 0.97 (t, 3H, CH₃); ¹³C NMR (CDCl₃): δ, 168.3; 135.3; 134.8; 134.6; 133.9; 133.3; 130.9; 130.5; 130.0; 129.9; 129.4; 127.4; 127.0; 42.1; 32.9; 23.2; 11.9. Cytotoxicity (MTT assay): IC₅₀=63.3±1.3 μM.

### Example 21—Synthesis of (Z)-3-(2-chlorophenyl)-2-((cyclohexylthio)methyl)acrylic acid (INF86)

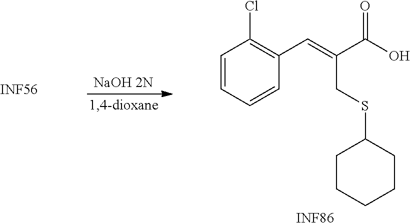 [SMILES: "INF56" ; O=C(O)/C(=C/c1ccccc1Cl)CSC1CCCCC1 ; "INF86"]

A solution of 2N NaOH (3 mL) is added to a solution of compound INF56 (0.0401 g; 0.118 mmol) in 1,4-dioxane (3 mL), and the reaction mixture is left under stirring at 20° C. for 16 hours. The mixture is acidified to pH=1 with 2N HCl (10 mL), and then extracted with EtOAc (3×50 mL). The combined organic phases are washed with a NaCl saturated solution (20 mL), dried (Na₂SO₄), and the solvent is evaporated under low pressure to obtain INF86 as a colourless oil (0.0367 g; yield: 95%). Negative ESI/MS: 309-311 [M−H]⁻; ¹H-NMR (CDCl₃): δ, 10.42 (br, 1H, COOH); 7.97 (s, 1H, C═CH); 7.65-7.28 (m, 4H, ArH); 3.58 (s, 2H, CH₂S); 2.60-2.58 (m, 1H, CH); 1.84-1.21 (m, 10H, cyclohexyl); ¹³C-NMR (CDCl₃): δ, 172.4; 139.0; 134.3; 133.5; 130.9; 130.5; 130.2; 129.7; 126.8; 43.9; 33.3; 26.2; 26.1; 25.8. Cytotoxicity (MTT assay): IC₅₀>100 μM.

### Example 22—Synthesis of ethyl 2-(2-chlorobenzyl)-3-(cyclohexylthio)propanoate (INF110)

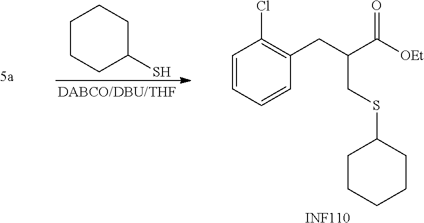 [SMILES: SC1CCCCC1 ; CCOC(=O)C(CSC1CCCCC1)Cc1ccccc1Cl ; "INF110"]

DABCO (0.628 g; 5.60 mmol), DBU (0.835 mL; 5.60 mmol) and cyclohexanethiol (0.822 mL; 6.72 mmol) are added in succession to a solution of 5a (0.630 g; 2.80 mmol) in anhydrous THF (20 mL), maintained at 20° C. in an inert atmosphere (N₂). The reaction mixture is kept under stirring at 20° C. for 4 hours. The mixture is diluted with CH₂Cl₂ (15 mL) and treated with 1N HCl (25 mL), and the two phases are separated. The aqueous phase is further extracted with CH₂Cl₂ (3×15 mL), and the combined organic phases are washed with a NaCl saturated solution (30 mL), dried (Na₂SO₄), and evaporated under low pressure. The resulting crude compound is purified by flash chromatography on silica gel column using PE/EtOAC 97/3 as eluent, followed by PE/EtOAC 95/5, to provide INF110 as a colourless oil (0.247 g; yield: 51%). MS/ESI m/z: 341-343 [M+H]⁺; ¹H-NMR (CDCl₃): δ, 7.40-6.98 (m, 41H, ArH); 4.00 (q, 1H, J=7.1 Hz, CH₂); 3.17-2.85 (m, 3H); 2.83-2.47 (m, 3H); 1.85 (dd, 211, J=9.6; 8.3 Hz); 1.68 (dd, 2H, J=9.1; 6.7 Hz); 1.53-1.43 (m, 1H); 1.36-1.15 (m, 5H); 1.06 (t, 3H, J=7.1 Hz, CH₃); ¹³C-NMR (75 MHz, CDCl₃): δ, 174.4; 136.8; 134.5; 131.7; 129.9; 128.5; 127.1; 61.0; 46.5; 44.2; 36.2; 34.0; 32.1; 26.4; 26.2; 14.5. Cytotoxicity (MTT assay): IC₅₀ 66.0±1.4 μM.

### Example 23—Synthesis of ethyl 2-(2-chlorobenzyl)-3-(cyclohexylsulphonyl)propanoate (INF111)

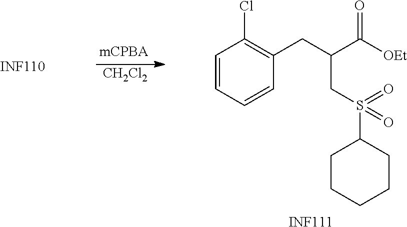 [SMILES: "INF110" ; "CH2Cl2" ; CCOC(=O)C(Cc1ccccc1Cl)CS(=O)(=O)C1CCCCC1 ; "INF111"]

75% mCPBA (0.890 g; 3.87 mmol) is added to a solution of compound INF110 (0.438 g; 1.29 mmol) in CH₂Cl₂ (15 mL), and the reaction mixture is left under stirring at 20° C. for 16 hours. The mixture is diluted with CH₂Cl₂ (15 mL), and extracted with a 10% solution of NaHCO₃ (3×15 mL). The organic phase is washed with a NaCl saturated solution (20 mL) and dried (Na₂SO₄), and the solvent is evaporated under low pressure. The resulting crude compound is purified by flash chromatography on silica gel column using PE/EtOAC 8/2 as eluent, to provide INF111 as a colourless oil (0.409 g; yield: 85%). MS/ESI m/z: 373-375 [M+H]⁺; ¹H-NMR (CDCl₃): δ, 7.38-7.21 (m, 1H); 7.17-7.03 (m, 3H); 4.12-3.96 (m, 2H, CH₂); 3.55-3.24 (m, 2H); 3.15-2.81 (m, 3H); 2.79-2.58 (in, 1H); 2.18-1.57 (m, 6H); 1.47-1.13 (m, 4H); 1.05 (t, 3H, J=7.2 Hz, CH₃). ¹³C-NMR (CDCl₃): δ, 173.2; 135.3; 134.7. 131.7; 130.2; 129.1; 127.4; 61.9; 61.8; 50.4; 39.8; 36.4; 25.4; 25.3; 25.2; 14.3. Cytotoxicity (MTT assay): IC₅₀ 98.1±4.8 μM.

### Example 24—Synthesis of ethyl 2-(2-chlorobenzyl)-3-(pyrrolidine-1-yl)propanoate (INF61)

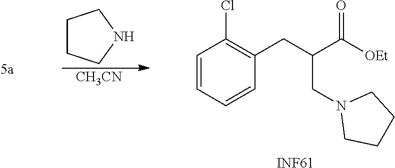

Pyrrolidine is added to a solution of 5a (0.838 g; 3.730 mmol) in CH₃CN (10 mL), and the reaction mixture is left under stirring at 20° C. for 72 hours. The solvent is evaporated under low pressure and the crude residue obtained is purified by flash chromatography on silica gel column, eluting with CH₂Cl₂/MeOH 98/2 to provide INF61 as a pale yellow oil (0.836 g; yield: 80%). CI-MS (isobutane) m/z: 296-298 [M+1]⁺; ¹H-NMR (DMSO-D6): δ, 7.32-7.10 (m, 4H, Ar—H); 4.05-3.98 (m, 2H, OCH₂CH₃); 3.12-2.81 (m, 5H, CH₂CHCH₂N); 2.58-2.49 (m, 4H, Pyr-H); 1.72 (m, 4H, Pyr-H); 1.05 (t, 3H, J=7.2 Hz, CH₃). ¹³C-NMR (CDCl₃): δ, 175.1; 137.4; 131.6; 129.8; 128.2; 127.0; 60.6; 58.6; 54.5; 45.9; 35.0; 30.1; 24.0; 14.5. Cytotoxicity (MTT assay): IC₅₀>100 μM.

### Example 25—Synthesis of ethyl 3-(2-chlorophenyl)-3-hydroxy-2-(pyrrolidin-1-ylmethyl)propanoate (INF37)

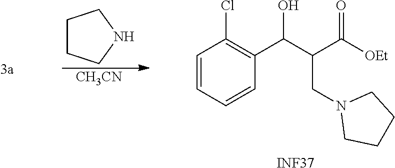

Pyrrolidine (0.103 g; 1.45 mmol) is added to a solution of 3a (0.264 g; 1.10 mmol) in CH₃CN (2 mL), and the reaction mixture is left under stirring at 20° C. for 24 hours. The solvent is evaporated under low pressure and the crude residue is taken up with CH₂Cl₂ (25 mL), washed with H₂O (2×20 mL) and dried (Na₂SO₄). The resulting oily residue is purified by flash chromatography on silica gel column, eluting with CH₂Cl₂/MeOH 99/1 (+0.1% Et₃N) to provide INF37 as a pale green oil (0.246 g; yield: 72%). Two fractions are obtained, identified as:

INF37 (syn). CI-MS (isobutane) m/z: 312-314 [M+1]⁺; ¹H-NMR (CDCl₃): δ, 7.63 (d, J=7.4 Hz, 1H, Ar—H); 7.40-7.17 (m, 3H, Ar—H); 5.57 (d, J=13.2 Hz, CHOH); 3.96 (m, 2H, OCH₂CH₃); 3.36-3.29 (m, 1H, CHCH₂N); 3.05-2.98 (m, 1H, CHCHHN); 2.82-2.61 (m, 5H, CHCHHN, 4 Pyr-H); 1.80 (m, 4H, Pyr-H); 1.01 (t, 31H, J=7.1 Hz, CH₃). ¹³C-NMR (CDCl₃): δ, 171.4; 139.6, 132.5; 129.3; 128.8; 128.6; 126.9; 73.7; 60.6; 56.9; 51.8; 49.1; 23.4; 13.7. Cytotoxicity (MTT assay): IC₅₀>100 μM.

INF37 (anti). CI-MS (isobutane) m/z: 312-314 [M+1]⁺; ¹H-NMR (CDCl₃): δ, 7.50 (d, J=8.5 Hz, 1H, Ar—H); 7.41-7.00 (m, 3H, Ar—H); 5.42 (d, J=17.2 Hz, CHOH); 3.98 (m, 2H, OCH₂CH₃); 3.41-3.21 (m, 114, CHCH₂N); 3.16-3.01 (m, 1H, CHCHHN); 3.03-2.86 (m, 114, CHCHHN); 2.83-2.62 (m, 4H, Pyr-H); 1.83 (m, 4H, Pyr-H); 1.03 (t, 3H, J=7.2 Hz, CH₃). ¹³C-NMR (CDCl₃): δ, 172.7; 139.6; 131.7; 129.3; 128.8; 127.9; 126.8; 70.1; 60.7; 55.0; 54.4; 48.6; 23.5; 13.9. Cytotoxicity (MTT assay): IC₅₀>100 μM.

### Example 26—Synthesis of ethyl (Z)-1-(3-(2-chlorophenyl)-2-((phenylthio)methyl)acryloyl)piperidine-3-carboxylate (INF176)

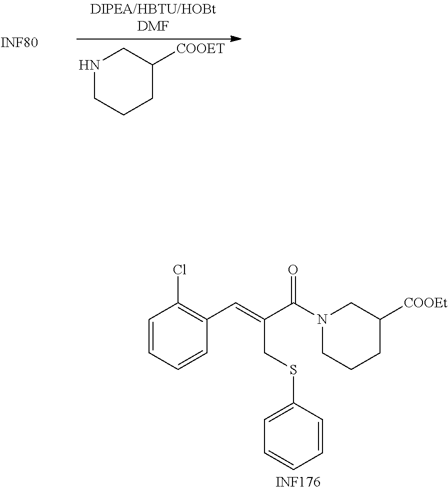

DIPEA (0.111 mL; 0.66 mmol), HOBt (4.43 mg; 0.03 mmol) and HBTU (0.187 g; 0.49 mmol) are added to a solution of INF80 (0.100 g; 0.33 mmol) in DMF (5 mL), and ethyl nipecotate (0.050 mL; 0.33 mmol) is added after 30 minutes. The reaction mixture is left to react under magnetic stirring for 18 hours at 20° C. The mixture is diluted with 15 mL of diethyl ether and the organic phase is washed with 1N HCl (3×15 mL) and NaCl saturated solution (15 mL), and dried (Na₂SO₄). The solvent is evaporated under low pressure, and the resulting residue is purified by flash chromatography on silica gel column, eluting with PE/EtOAc 9/1 followed by PE/EtOAc 8/2. INF176 is obtained as a pale yellow oil (0.100 g; yield: 69%). The purity (HPLC) is 96%; eluent CH₃CN/1120+0.1% CF₃COOH, 70/30; flow rate 1.0 mL/min; t_{R}=8.558. Rf=0.75. (PE/EtOAc/MeOH 7.5:2:0.5); MS (ESI) m/z: 444-446 [M+H]⁺; ¹H-NMR (DMSO-D6, 80° C.): δ, 7.47-7.46 (m, 1H, Ar—H); 7.35-7.33 (m, 3H, Ar—H); 7.16-7.08 (m, 5H, Ar—H); 6.61 (s, 1H, C═CH); 4.20-4.18 (m, 1H, CH-pip); 4.04 (q, J=7.1 Hz; 2H, O—CH₂); 3.91-3.86 (m, 2H, S—CH₂); 3.08-2.93 (m, 2H, N—CH₂ₚᵢₚ); 2.44 (m, 2H, CH₂ₚᵢₚ); 1.96-1.94 (m, 1H, CH₂ₚᵢₚ); 1.66-1.57 (m, 2H, CH₂ₚᵢₚ); 1.41-1.39 (m, 1H, CH₂ₚᵢₚ); 1.23 (t, J=7.1 Hz; 3H, CH₃). ¹³C-NMR (CDCl₃): δ, 172.6; 169.6; 135.3; 134.4; 133.4; 130.5; 129.65; 129.58; 129.2; 129.0; 128.5; 128.4, 126.8; 126.0; 60.7; 48.2*; 43.9; 41.3; 31.8; 27.7*; 24.8*; 14.3. *peaks doubled due to the presence of rotational isomers (rotamers). Cytotoxicity (MTT assay): IC₅₀ 94.2±6.9 μM.

### Example 27—Synthesis of ethyl (R,Z)-1-(3-(2-chlorophenyl)-2-((phenylthio)methyl)acryloyl)piperidine-3-carboxylic acid (INF202)

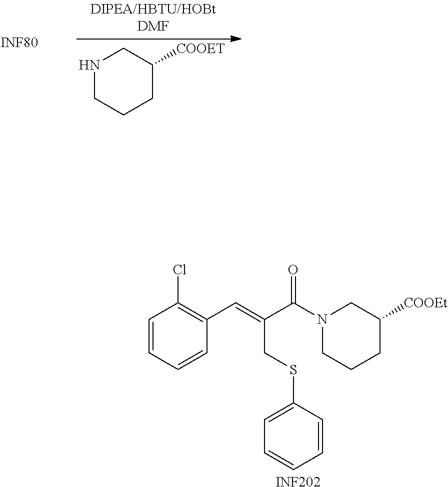

Compound INF202 is obtained by following the same procedure as described in Example 26 for INF176, using INF80 (0.08 g; 0.262 mmol), DIPEA (0.088 mL; 0.525 mmol), HOBt (4.00 mg; 0.03 mmol), HBTU (0.149 g; 0.393 mmol) and (R)-ethyl nipecotate (0.044 mL; 0.288 mmol). INF202 is obtained (0.100 g; yield: 88%). ESI/MS m/z: 444-446 [M+H]⁺; ¹H-NMR (CDCl₃): δ, 7.41-7.38 (m, 1H, Ar—H); 7.31 (m, 1H, Ar—H); 7.26 (m, 2H, Ar—H); 7.15-7.06 (m, 5H, Ar—H); 6.58 (s, 1H, C═CH); 4.49 (m, 2H, 0-CH₂); 3.92 (dd, J=23.9; 7.3 Hz, 2H, N—CH₂); 3.08-2.93 (m, 2H, S—CH₂); 2.61 (m, 2H, N—CH₂ₚᵢₚ); 2.41 (dd, J=49.7; 24.3 Hz, 1H, CH); 1.80-1.66 (m, 2H, CH₂ₚᵢₚ); 1.63-1.40 (m, 2H, CH₂ₚᵢₚ); 1.23 (m, 3H).

### Example 28—Synthesis of ethyl (S,Z)-1-(3-(2-chlorophenyl)-2-((phenylthio)methyl)acryloyl)piperidine-3-carboxylate (INF203)

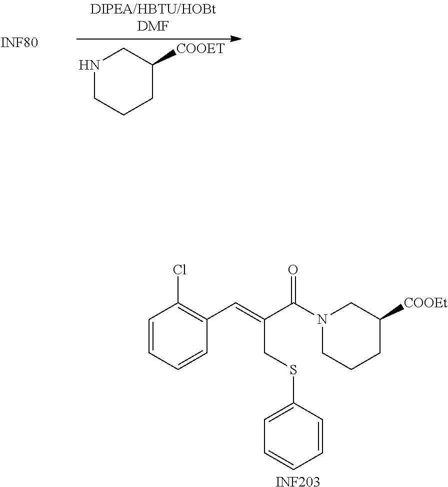

Compound INF203 is obtained by following the same procedure as described in Example 25 for INF176, using INF80 (0.08 g; 0.262 mmol), DIPEA (0.088 mL; 0.525 mmol), HOBt (4.00 mg; 0.03 mmol), HBTU (0.149 g; 0.393 mmol) and (S)-ethyl nipecotate (0.044 mL; 0.288 mmol). INF203 is obtained (0.086 g; yield: 74%). MS (ESI) m/z: 444-446 [M+H]⁺; ¹H-NMR (CDCl₃): δ, 7.41-7.38 (m, 1H, Ar—H); 7.31 (m, 1H, Ar—H); 7.26 (m, 2H, Ar—H); 7.15-7.06 (m, 5H, Ar—H); 6.58 (s, 1H, C═CH); 4.49 (m, 2H, O—CH₂); 3.92 (dd, J=23.9; 7.3 Hz, 2H, N—CH₂); 3.08-2.93 (m, 2H, S—CH₂); 2.61 (m, 2H, N—CH₂ₚᵢₚ); 2.41 (dd, J=49.7; 24.3 Hz, 1H, CH); 1.80-1.66 (m, 2H, CH₂ₚᵢₚ); 1.63-1.40 (m, 2H, CH₂ₚᵢₚ); 1.23 (n, 3H).

### Example 29—Synthesis of (Z)-1-(3-(2-chlorophenyl)-2-((phenylthio)methyl)acryloyl)piperidine-3-carboxylic acid (INF177)

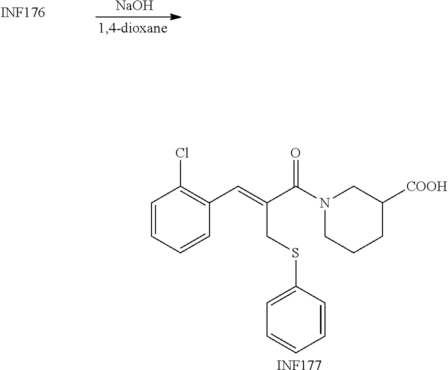

2.5M NaOH (0.500 mL) is added to a solution of INF176 (0.047 g; 0.110 mmol) in dioxane (1 mL), and the reaction is laced under stirring at 20° C. for 18 hours. When said time has elapsed, 1N HCl (5 mL) and H₂O (5 mL) are added, and the reaction mixture is extracted with CH₂Cl₂ (3×15 mL) and NaCl saturated solution (15 mL), and dried (Na₂SO₄). The solvent is evaporated under low pressure, and the crude compound is purified by flash chromatography on silica gel column, eluting with CH₂Cl₂/MeOH 95/5. INF177 is obtained as a rubbery solid (0.042 g; yield: 96%). Purity (HPLC): 98% eluent CH₃CN/H₂O+0.1% CF₃COOH, 60/40; flow rate 1.0 mL/min; t_{R}=7.303. Rf=0.2 (DCM/MeOH 95/5); Negative ESI/MS m/z: 414-416 [M−H]⁻; ¹H-NMR (CD₃OD): δ, 7.70 (s, 1H, ArH); 7.47 (d, J=12.1 Hz, 2H, ArH); 7.34 (d, J=12.4 Hz, 2H, ArH); 7.28-7.17 (m, 4H, ArH); 7.09 (s, 1H, C═CH); 4.07 (s, 1H, CH); 3.68 (s, 2H, S—CH₂); 3.62 (t, J=5.9 Hz, 3H, Pip-H); 3.41-3.15 (m, 2H, Pip-H); 2.29-1.87 (m, 3H, Pip-H). ¹³C-NMR (CD₃OD): δ, 178.0; 170.2; 134.0; 133.2; 131.8; 130.4; 129.5; 129.4; 129.3; 129.1; 128.9; 128.6; 126.7; 125.9; 39.4; 31.8; 31.6; 29.6; 27.1; 22.6. Cytotoxicity (MTT assay): IC₅₀>100 μM.

### Example 30—Synthesis of ethyl (Z)-1-(3-(2-chlorophenyl)-2-((phenylthio)methyl)acryloyl)piperidine-4-carboxylate (INF180)

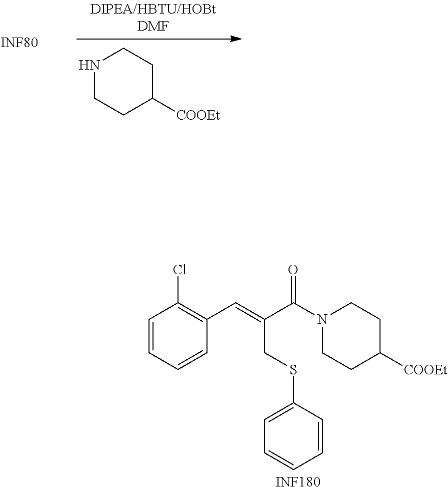

DIPEA (0.322 mL; 1.90 mmol), HOBt (0.013 g; 0.095 mmol) and HBTU (0.539 g; 1.42 mmol) are added to a solution of INF80 (0.289 g; 0.95 mmol) in DMF (15 mL), and ethyl isonipecotate (0.146 mL; 0.95 mmol) is added after 30 minutes. The reaction mixture is left to react under magnetic stirring for 16 hours at 20° C. The reaction mixture is diluted with 15 mL of diethyl ether, and the organic phase is washed with 1N HCl (3×15 mL) and NaCl saturated solution (15 mL), dried (Na₂SO₄), and the solvent evaporated under low pressure. The resulting crude compound is purified by flash chromatography on silica gel column, eluting with PE/EtOAc 8/2. INF180 is obtained as a rubbery solid (0.218 g; yield: 52%). Purity (HPLC): 97%; eluent CH₃CN/H₂O+0.1% CF₃COOH, 70/30; flow rate 1.0 mL/min; t_{R}=7.947. Rf=0.7 (PE/EtOAc/MeOH 7:2:1); MS (ESI) m/z: 444-446 [M+H]⁺; ¹H-NMR (CDCl₃): δ, 7.41-7.38 (m, 1H, Ar—H); 7.36-7.33 (m, 1H, Ar—H); 7.28 (d, J=3.5 Hz, 1H, Ar—H); 7.26 (d, J=12.3 Hz, 1H, Ar—H); 7.13 (t, J=7.2 Hz, 2H, Ar—H); 7.10-7.07 (m, 1H, Ar—H); 7.05 (d, J=7.9 Hz, 2H, Ar—H); 6.59 (s, 1H, C═CH); 4.16-4.08 (m, 3H, Hₚᵢₚ); 4.03 (s, 2H, Hₚᵢₚ); 3.00 (s, 2H, S—CH₂), 2.55-2.45 (m, 1H, CH); 1.99-1.85 (m, 2H, Hₚᵢₚ); 1.80 (s, 1H, Hₚᵢₚ); 1.67 (s, 2H, Hₚᵢₚ); 1.25 (dd, J=9.5; 4.7 Hz, 3H, CH₃). ¹³C-NMR (CDCl₃): δ, 174.3; 169.9; 135.4; 134.41; 134.36; 133.50; 130.58; 129.7; 129.6; 129.0; 128.6; 128.5; 126.9; 126.2; 60.7; 47.0; 41.1; 32, 8; 27.9; 14.3. Cytotoxicity (MTT assay): IC₅₀>100 μM.

### Example 31—Synthesis of (Z)-1-(3-(2-chlorophenyl)-2-((phenylthio)methyl)acryloyl)piperidine-4-carboxylic acid (INF187)

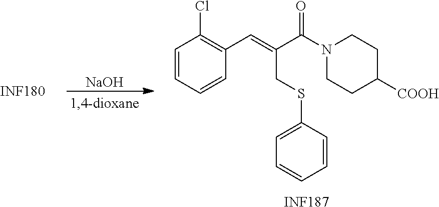

2.5M NaOH (1.14 mL) is added to a solution of INF180 (0.107 g; 0.24 mmol) in dioxane (2 mL), and the reaction is conducted at 20° C. for 18 hours under stirring. The reaction mixture is diluted with 1N HCl (5 mL) and H₂O (5 mL), and extracted with CH₂Cl₂ (3×15 mL) and NaCl saturated solution (15 mL), dried (Na₂SO₄), and the solvent evaporated under low pressure. The resulting crude compound is purified by flash chromatography on silica gel column, eluting with CH₂Cl₂/MeOH 95/5. INF187 is obtained as a pale yellow oil (0.062 g; yield: 62%). Purity (HPLC): 98.6%; eluent CH₃CN/H₂O+0.1% CF₃COOH, 60/40; flow rate 1.0 mL/min; t_{R}=6.416. Rf=0.3 (DCM/MeOH 95/5); Negative ESI/MS m/z: 414-416 [M−H]⁻; ¹H-NMR (CD₃OD): δ, 8.81 (t, J=8.5 Hz, 1H, ArH); 8.74-8.69 (m, 3H, ArH); 8.51-8.44 (m, 3H, ArH); 8.42-8.32 (m, 2H, ArH); 7.97 (s, 1H, C═CH); 5.69 (d, J=10.7 Hz, 1H, CH); 5.35 (d, J=13.1 Hz, 3H, Pip-H); 4.64 (s, 2H, S—CH₂); 4.33-3.62 (m, 1H, Pip-H); 3.35-2.87 (m, 411, Pip-H). ¹³C-NMR (CD₃OD): δ, 176.8; 170.3; 134.9; 134.2; 134.0; 133.2; 130.4; 129.7; 129.4; 129.3; 128.8; 128.6; 126.8; 126.0; 41.3; 40.6; 30.9; 27.7. Cytotoxicity (MTT assay): IC₅₀>100 μM.

### Example 32—Synthesis of 2-(2-chlorobenzyl)-3-(phenylthio) propanoic acid

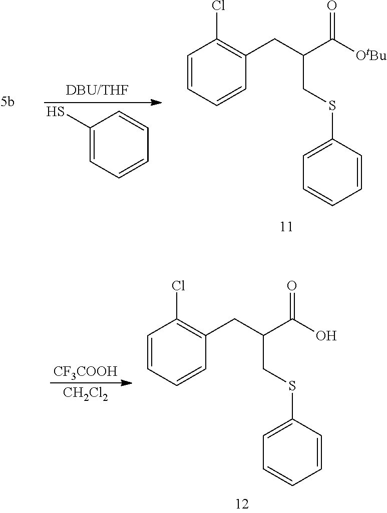 [SMILES: Sc1ccccc1 ; CC(C)(C)OC(=O)C(CSc1ccccc1)Cc1ccccc1Cl ; "CF3COOH" ; "CH2Cl2" ; O=C(O)C(CSc1ccccc1)Cc1ccccc1Cl]

DBU (1.21 mL; 8.07 mmol) and thiophenol (0.988 mL; 9.69 mmol) are added to a solution of 5b (1.02 g; 4.04 mmol) in THE (42 mL), maintained under an inert atmosphere (N₂). The reaction mixture is left under magnetic stirring for 4 hours at 20° C. The solvent is evaporated under low pressure, the resulting residue is taken up with CH₂Cl₂ (10 mL), and the organic phase is washed with 1N HCl (3×20 mL) and NaCl saturated solution (15 mL), dried (Na₂S04), and the solvent evaporated under low pressure. The resulting crude compound is purified by flash chromatography on silica gel column, eluting with PE/DCM 8/2. The intermediate tert-butyl 2-(2-chlorobenzyl)-3-(phenylthio) propanoate (11) is obtained as a colourless oil (1.12 g; yield: 77%). Rf=0.43 (PE/EtOAc 95/5); ESI/MS m/z: 363-365 [M+H]⁺. Intermediate 11, characterised by mass spectrometry, is used directly in the next step.

CF₃COOH (6 mL; 78.4 mmol) is added to a solution of 11 (1.08 g; 2.99 mmol) in CH₂Cl₂ (60 mL), and the reaction mixture is left under magnetic stirring at 16° C. for 20 hours. The organic phase is washed with H₂O (2×15 mL) and NaCl saturated solution (15 mL), and dried (Na₂SO₄). After evaporation of the solvent under low pressure, 2-(2-chlorobenzyl)-3-(phenylthio) propanoic acid (12) is obtained (0.812 g; yield 89%) as pure compound. Negative ESI/MS m/z: 305-307 [M−H]⁻; ¹H-NMR (CDCl₃): δ, 8.45 (s, 1H, OH); 6.93-6.90 (m, 1H, Ar—H); 6.87 (t, J=1.7 Hz, 1H, Ar—H), 6.85 (t, J=1.6 Hz, 1H, Ar—H); 6.83 (d, J=1.7 Hz, 1H, Ar—H); 6.81 (dd, J=8.1; 1.7 Hz, 1H, Ar—H); 6.79-6.76 (m, 1H, Ar—H); 6.76 (t, J=1.7 Hz, 1H, Ar—H); 6.75 (s, 1H, Ar—H); 6.75-6.72 (m, 1H, Ar-1H); 2.81-2.76 (m, 1H, CH); 2.69 (dd, J=9.2; 4.6 Hz, 2H, CH₂); 2.68-2.64 (m, 2H, CH₂). ¹³C-NMR (CDCl₃): δ, 179.5; 135.8; 135.3; 134.37; 131.41; 130.2; 129.9; 129.1; 128.5; 127.0; 126.8; 45.4; 35.4; 35.2.

### Example 33—Synthesis of ethyl 1-(2-(2-chlorobenzyl)-3-(phenylthio)propanoyl)piperidine-3-carboxylate (INF184 and INF185)

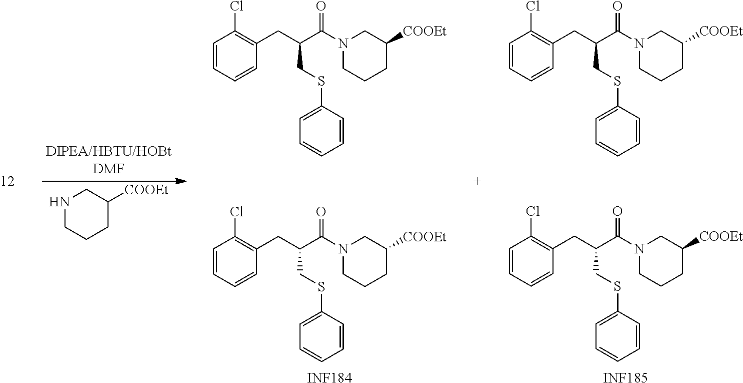

DIPEA (0.163 mL; 0.96 mmol), HOBt (6.47 mg; 0.05 mmol) and HBTU (0.273 g; 0.72 mmol) are added to a solution of 12 (0.146 g; 0.48 mmol) in DMF (7.3 mL). Ethyl nipecotate (0.075 mL; 0.48 mmol) is added after 30 minutes. The reaction mixture is left under magnetic stirring for 18 hours at 20° C. The mixture is diluted with 15 mL of diethyl ether, and the organic phase is washed with 1N HCl (3×15 mL) and NaCl saturated solution (15 mL), and dried (Na₂SO₄). The solvent is evaporated under low pressure, and the crude compound is purified by flash chromatography on silica gel column, eluting with PE/EtOAc 8.5/1.5. A mixture of INF184 and INF185 is obtained as a yellow oil (68 mg; yield: 32%).

The mixture of isomers is isolated in two different aliquots; each of which is more concentrated than one of the two diastereomers. The 1st aliquot consists of 8% isomer INF184 and 92% INF185. The 2nd aliquot consists of 86% isomer INF184 and 14% INF185; eluent CH₃CN/H₂O+0.1% CF₃COOH, 65/35; flow rate 1.0 mL/min. The retention times are 17.440 minutes (INF184) and 18.405 minutes (INF185) respectively.

INF184+INF185 mixture of isomers: 1H-NMR (CDCl₃): δ, 7.33-7.22 (m, 7H, ArH); 7.22-7.09 (m, 1H, ArH); 4.58 (m, 2H, CH₂); 4.14-3.96 (m, 4H, CH₂); 3.64 (d, J=13.5 Hz, 2H, CH₂); 3.46 (m, 2H, CH₂); 3.32 (m, 2H, CH); 3.17-3.01 (m, 4H, CH₂); 3.02-2.90 (m, 2H, CH); 2.67-2.55 (m, 2H, CH₂); 2.41-2.26 (m, 2H, CH₂); 2.22-2.14 (m, 2H, CH₂); 2.07-1.90 (m, 2H, CH₂); 1.78-1.59 (m, 2H, CH₂); 1.47-1.28 (m, 4H, CH₂); 1.25-1.18 (m, 6H, CH₃). ¹³C-NMR (CDCl₃): δ, 173.41; 172.94, 172.37; 172.22; 136.85; 136.47; 136.15; 136.10; 134; 35; 134.24; 132.47; 132.00; 129.87; 129.83; 129.34; 129.27; 128.68; 128.64; 127.25; 127.17; 126.9; 126.44; 126.11; 60.94; 60.83; 47.50; 46.41; 44.22; 42.83; 42.33; 41.64; 40.18; 40.13; 37.73; 37.59; 36.62; 35.34; 27.87; 27.51; 24.88; 24.78; 14.50; 14.47. Cytotoxicity (MTT assay): IC₅₀ 75.4±2.6 μM.

### Example 34—Synthesis of ethyl 1-(2-(2-chlorobenzyl)-3-(phenylthio)propanoyl)piperidine-4-carboxylate (INF186)

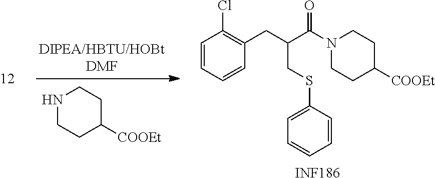

DIPEA (0.157 mL; 0.92 mmol), HOBt (6.21 mg; 0.05 mmol) and HBTU (0.261 g; 0.69 mmol) are added to a solution of 12 (0.140 g; 0.46 mmol) in DMF (7.00 mL). After 30 minutes under stirring at 20° C., ethyl isonipecotate (0.070 mL; 0.46 mmol) is added, and the mixture is left under stirring at 20° C. for 18 hours. The mixture is diluted with diethyl 5 ether (15 mL), and the organic phase is washed with 1N HCl (3×15 mL) and NaCl saturated solution (15 mL), and dried (Na₂SO₄). The solvent is evaporated under low pressure, and the crude residue is purified by flash chromatography on silica gel column, eluting with DCM/EtOAc 98/2); INF186 is thus obtained as a pale yellow oil (0.126 g; yield: 62%). Purity (HPLC)>99%; eluent CH₃CN/H₂O+0.1% CF₃COOH, 60/40; flow rate 1.0 mL/min; t_{R}=16.019. Rf=0.23 (DCM/EtOAc 98/2); MS (ESI) m/z: 446-448 [M+H]⁺; ¹H-NMR (CDCl₃): δ, 7.86-6.66 (m, 9H, ArH); 4.44-4.00 (m, 2H, CH₂); 3.59-3.25 (m, 2H, CH₂); 3.30-2.91 (m, 2H, CH₂); 2.88-2.44 (m, 111, CH); 2.25 (dd, J=12.2; 9.6 Hz, 1H, CH); 2.16 (s, 6H, CH₂); 1.77-1.30 (m, 2H, CH₂); 1.26-1.08 (m, 3H, CH₃). ¹³C-NMR (CDCl₃): δ, 174.0; 171.9; 135.9; 132.2; 132.0; 129.7; 129.1; 129.0; 128.9; 127.2; 127.0; 126.2; 60.7; 45.3; 41.6; 39.9; 37.4; 36.2; 27.9; 14.3. Cytotoxicity (MTT assay): IC₅₀ 94.6±4.0 μM.

### Example 35—Synthesis of 2-(2-chlorobenzyl)-3-(cyclohexylthio) propanoic acid (14)

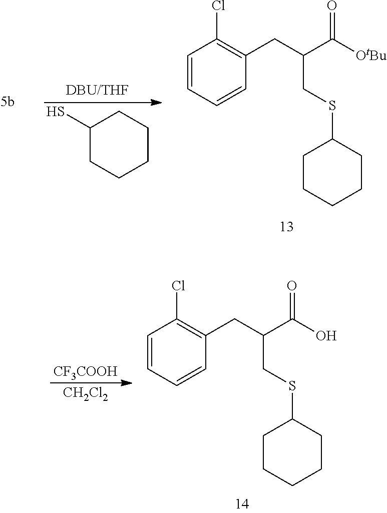 [SMILES: SC1CCCCC1 ; CC(C)(C)OC(=O)C(CSC1CCCCC1)Cc1ccccc1Cl ; "CF3COOH" ; "CH2Cl2" ; O=C(O)C(CSC1CCCCC1)Cc1ccccc1Cl]

DBU (2.18 mL; 14.7 mmol) and cyclohexanethiol (1.16 mL; 11.8 mmol) are added to a solution of 5b (1.06 g; 4.19 mmol) in CH₂Cl₂ (11 mL), maintained under an inert atmosphere (N₂); the reaction mixture is left under magnetic stirring at 20° C. for 18 hours. The reaction mixture is treated with H₂O (15 mL) and CH₂Cl₂ (15 mL), and the phases are separated. The organic phase is washed with 1N HCl (3×15 mL) and NaCl saturated solution (15 mL), and dried (Na₂SO₄). The solvent is evaporated under low pressure, and the crude residue is purified by flash chromatography on silica gel column, eluting with PE/DCM 8/2. The intermediate tert-butyl 2-(2-chlorobenzyl)-3-(cyclohexylthio) propanoate is thus obtained (13; 0.544 g; yield: 35%). Rf=0.6 (PE/DCM 7/3); MS (ESI) m/z: 369-371 [M+H]⁺; ¹H-NMR (CDCl₃): δ, 7.33 (d, J=3.6 Hz, 1H, ArH); 7.26-7.20 (in, 1H, ArH); 7.14 (t, J=7.3 Hz, 2H, ArM); 3.08-3.01 (m, 1H, CH₂—CH—CH₂); 2.97-2.86 (m, 2H, CH₂); 2.79 (dd, J=12.5; 8.0 Hz, 1H, S—CH); 2.69-2.58 (m, 2H, CH₂); 1.91 (d, J=12.9 Hz, 2H, CH₂); 1.73 (s, 2H, CH₂); 1.59 (d, J=10.6 Hz, 2H); 1.33 (s, 9H, CH₃); 1.30-1.20 (m, 4H, cyclohexyl-H). ¹³C-NMR (CDCl₃): δ, 173.6; 137.0; 134.5; 131.8; 129.9; 128.3; 126.9; 81.2; 47.0; 44.1; 36.3; 33.9; 32.4; 28.3; 26.4; 26.2.

CF₃COOH (3 mL; 39.6 mmol) is added to a solution of 13 (0.522 g; 1.42 mmol) in CH₂Cl₂ (30 mL), and the reaction mixture is left under magnetic stirring at 20° C. for 16 hours. The organic phase is washed with 1N HCl (2×15 mL) and NaCl saturated solution (15 mL), and dried (Na₂SO₄). The solvent is evaporated under low pressure and the crude residue is purified by flash chromatography on silica gel column, eluting with PE/EtOAc 7/3) to obtain 14 as a yellow oil (0.348 g; yield 79%). Negative ESI/MS M/z: 311-313 [M−H]⁻; ¹H-NMR (CDCl₃): δ, 8.52 (s, 1H, OH); 7.34 (dd, J=5.5; 3.6 Hz, 1H, Ar—H); 7.26-7.21 (m, 1H, Ar—H); 7.17 (dd, J=5.6; 3.5 Hz, 2H, Ar—H); 3.14-3.06 (m, 2H CH—CH₂); 3.03 (dd, J=13.4; 6.2 Hz, 1H, CH); 2.82 (dd, J=13.2; 7.6 Hz, 1H, CH—CH_{b}); 2.71 (dd, J=13.1; 4.7 Hz, 1H CH—CHₐ); 2.63 (s, 1H, S—CH); 1.94-1.81 (m, 2H, cyclohexyl-H); 1.73 (s, 2H, cyclohexyl-H); 1.58 (d, J=10.3 Hz, 2H, cyclohexyl-H); 1.31-1.16 (m, 4H, cyclohexyl-H). ¹³C-NMR (CDCl₃): δ, 179.7; 136.2; 134.3; 131.5; 129.8; 128.4; 127.0; 46.0; 44.0; 35.4; 33.6; 31.3; 26.2; 25.9.

### Example 36—Synthesis of ethyl 1-(2-(2-chlorobenzyl)-3-(cyclohexylthio)propanoyl)piperidine-3-carboxylate (INF192)

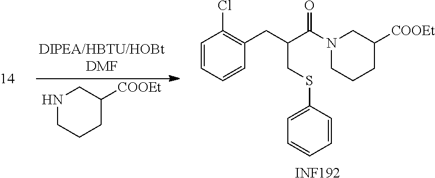

DIPEA (0.359 mL; 2.11 mmol), HOBt (0.014 g; 0.11 mmol) and HBTU (0.599 g; 1.58 mmol) are added to a solution of 14 (0.330 g; 1.06 mmol) in DMF (16 mL). After 30 minutes under stirring at 20° C., ethyl nipecotate (0.164 mL; 1.06 mmol) is added, and the mixture is left under stirring at 20° C. for 18 hours. The mixture is diluted with diethyl ether (15 mL), and the organic phase is washed with 1N HCl (3×15 mL) and NaCl saturated solution (15 mL), and dried (Na₂SO₄). The solvent is evaporated under low pressure and the crude residue is purified by flash chromatography on silica gel column, eluting with PE/EtOAc 9/1), followed by PE/EtOAc 8/2; INF192 is thus obtained as a pale yellow oil (0.279 g; yield: 59%). Purity (HPLC)>99%; eluent CH₃CN/H₂O+0.1% CF₃COOH, 80/20; flow rate 1.0 mL/min; t_{R}=13.237. Rf=0.33 (PE/EtOAc 8/2); MS (ESI) m/z: 452-454 [M+H]⁺; ¹H-NMR (CDCl₃): δ, 7.32 (t, J=6.8 Hz, 1H, ArH); 7.23 (d, J=32.3 Hz, 1H, ArH); 7.15 (d, J=3.0 Hz, 2H, ArH); 4.77-4.36 (m, 1H, CH); 4.11 (s, 2H, CH₂); 4.02-3.59 (m, 1H, CH); 3.41 (t, J=37.6 Hz, 1H, CH); 3.24-2.82 (m, 4H, CH₂); 2.76-2.27 (m, 5H, CH₂); 2.18-1.99 (m, 1H); 1.90 (dd, J=46.7; 33.2 Hz, 2H, CH₂); 1.84-1.64 (m, 3H, CH₂); 1.57 (t, J=25.2 Hz, 2H, CH₂); 1.53-1.38 (m, 2H, CH₂); 1.30-1.27 (m, 3H, CH₂); 1.25 (d, J=6.8 Hz, 3H, CH₃). ¹³C-NMR (CDCl₃): δ, 173.2; 172.4; 136.9; 134.2; 132.3; 131.8; 128.3; 126.9; 60.8; 47.9; 46.4; 44.5; 41.5; 37.7; 33.9; 33.6; 33.4; 27.2; 26.2; 25.9; 24.3; 14.3. Cytotoxicity (MTT assay): IC₅₀ 59.8±0.7 μM.

### Example 37—Synthesis of ethyl (Z)-1-(3-(2-chlorophenyl)-2-((cyclohexylthio)methyl)acryloyl)piperidine-4-carboxylate (INF188)

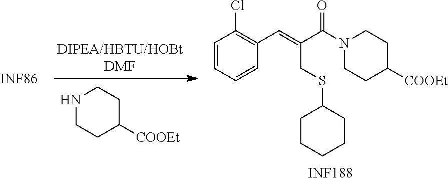

DIPEA (0.213 mL; 1.26 mmol), HOBt (8.48 mg; 0.06 mmol) and HBTU (0.357 g; 0.94 mmol) are added to a solution of INF86 (0.195 g; 0.63 mmol) in DMF (9.5 mL), and after 30 minutes under stirring at 20° C., ethyl isonipecotate (0.096 mL; 0.63 mmol) is added. The reaction mixture is left to react under magnetic stirring for 18 hours at 20° C. The mixture is diluted with diethyl ether (15 mL), and the organic phase is washed with 1N HCl (3×15 mL) and NaCl saturated solution (15 mL), and dried (Na₂SO₄). The solvent is evaporated under low pressure and the crude residue is purified by flash chromatography on silica gel column, eluting with PE/EtOAc 8.5/1.5); INF188 is thus obtained as a pale yellow oil (0.199 g; yield 70%). Purity (HPLC): 96%; eluent CH₃CN/H₂O+0.1% CF₃COOH, 70/30; flow rate 1.0 mL/min; t_{R}=17.767. Rf=0.5 (PE/EtOAc/MeOH 8/1.5/0.5); MS (ESI) m/z: 450-452 [M+H]⁺; ¹H-NMR (CDCl₃): δ, 7.42-7.38 (m, 3H, Ar—H); 7.38-7.34 (m, 3H, Ar—H); 7.29 (dd, J=7.4; 1.1 Hz, 1H, Ar—H); 7.19 (td, J=7.7; 1.6 Hz, 1H, Ar—H); 6; 56 (s, 2H, C═CH); 5.56-5.06 (m, 3H, CH₂); 4.16 (q, J=7.1 Hz, 5H, CH₂); 3.58 (s, 4H, S—CH₂); 3.20 (m, 2H₄, CHₚᵢₚ); 2.59 (m, 4H, CH₂); 2.50-2.44 (m, 4H, CH₂); 2.00 (d, J=11.6 Hz, 4H, CH₂); 1.91 (in, 6H, CH₃); 1.67 (s, 4H, CH₂); 1.54 (m, 4H, CH₂); 1.25 (m, 14H, CH₂). Cytotoxicity (MTT assay): IC₅₀ 58.6±5.3 μM.

### Example 38—Synthesis of ethyl 1-(2-(2-chlorobenzyl)-3-(cyclohexylthio)propanoyl)piperidine-4-carboxylate (INF193)

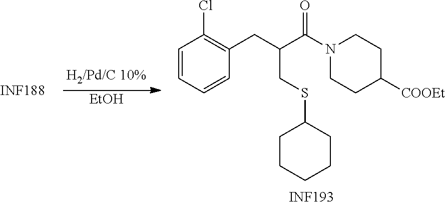

10% Pd/C (2.37 mg) is added to a solution of INF188 (0.100 g; 0.22 mmol) in absolute EtOH (2.6 mL), and the mixture is placed under H₂ atmosphere at the pressure of 1 bar. The reaction mixture is left under stirring at 20° C. for 18 hours. The catalyst is filtered through celite, and the filtrate is evaporated under low pressure to obtain INF193 as a yellow oil (0.090 g; yield 90%).

Purity (HPLC): 91%; eluent CH₃CN/H₂O+0.1% CF₃COOH, 70/30; flow rate 1.0 mL/min; t_{R}=18.238. Rf=0.2 (PE/EtOAc 9/1); MS (ESI) m/z: 452-454 [M+H]⁺; ¹H-NMR (CDCl₃): δ, 7.34-7.29 (m, 2H, ArH); 7.22-7.17 (m, 1H, ArH); 7.17-7.14 (m, 21H, ArH); 7.14 (dd, J=3.0; 0.8 Hz, 2H, ArH); 7.12-7.10 (m, II, ArH); 4.76-4.35 (m, 2H, CH₂); 4.20-4.04 (m, 4H, CH₂); 4.01-3.59 (m, 2H, CH); 3.52-3.28 (m, 2H, CH); 3.20-2.99 (m, 2H, CH); 2.99-2.80 (m, 5H, CH₂); 2.73-2.57 (m, 5H, CH₂); 2.54-2.28 (m, 3H, CH₂); 2.15-1.83 (m, 5H, CH₂); 1.81-1.62 (m, 71H, CH₂); 1.61-1.37 (m, 71H, CH₂); 1.27 (dd, J=13.2; 6.1 Hz, 7H, CH₂); 1.25-1.21 (m, 8H, CH₂); 1.05-0.95 (in, 1H, CH₂). Cytotoxicity (MTT assay): IC₅₀ 77.5±7.3 μM.

### Example 39—Synthesis of ethyl (Z)-1-(3-(2-chlorophenyl)-2-((cyclohexylthio)methyl)acryloyl)piperidine-3-carboxylate (INF194)

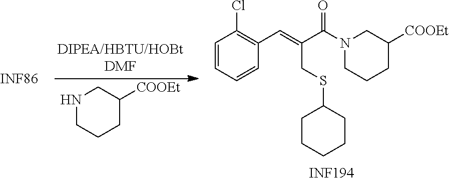

DIPEA (0.062 mL; 0.36 mmol), HOBt (2.43 mg; 0.02 mmol) and HBTU (0.102 g; 0.27 mmol) are added to a solution of INF86 (0.056 g; 0.18 mmol) in DMF (3 mL). After 30 minutes' stirring at 20° C., ethyl nipecotate (0.028 mL; 0.18 mmol) is added to the mixture, and the reaction mixture is left under stirring for 16 hours at 20° C. The mixture is diluted with diethyl ether (15 mL), and the organic phase is washed with 1N HCl (3×15 mL) and NaCl saturated solution (15 mL), and dried (Na₂SO₄). The solvent is evaporated under low pressure, and the crude residue is purified by flash chromatography on silica gel column, eluting with PE/EtOAc 8/2. INF194 is thus obtained as a rubbery solid (0.052 g; yield 64%). Purity (HPLC): 89%; eluent CH₃CN/H₂O+0.1% CF₃COOH, 80/20; flow rate 1.0 mL/min; t_{R}=11.690. Rf=0.5 (PE/EtOAc/MeOH 8/1.5/0.5); MS (ESI) m/z: 450-452 [M+H]⁺; ¹H-NMR (CDCl₃): δ, 7.41 (dd, J=7.6; 1.6 Hz, 1H, Ar—H); 7.31-7.25 (m, 3H, Ar—H); 6.59 (s, 1H, C═CH); 4.65 (s, 2H, CH₂); 4.19 (m, 3H, CH₂); 3.62 (t, J=14.2 Hz, 2H, CH₂); 3.19 (m, 2H, CH₂); 2.64 (m, 2H, CH₂); 2.45 (s, 1H, CH₂); 2.28-2.11 (m, 1H, S—CH), 1.88-1.82 (m, 1H, CH); 1.61 (d, J=5.5 Hz, 3H, CH₂); 1.52 (d, J=16.4 Hz, 2H, CH]₂); 1.26 (t, J=7.0 Hz, 3H, CH₃); 1.12 (m, 5H, CH₂). ¹³C-NMR (CDCl₃): δ, 173.0; 171.4; 134.9; 134.4; 134.0; 131.0; 129.8; 129.7; 127.8; 127.2; 61.1; 47.9; 45.6; 43.0; 41.6; 35.4; 33.5; 27.9; 26.4; 26.0; 22.2; 14.6. Cytotoxicity (MTT assay): IC₅₀ 78.3±10.2 μM.

### Example 40—Synthesis of (Z)-1-(3-(2-chlorophenyl)-2-((phenylthio)methyl)acryloyl)piperidine-3-carboxamide (INF219)

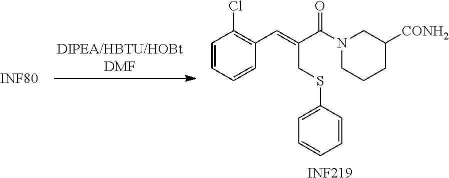

DIPEA (0.177 mL; 1.065 mmol), HOBt (0.005 g; 0.034 mmol) and HBTU (0.202 g; 0.532 mmol) are added to a solution of INF80 (0.108 g; 0.335 mmol) in DMF (3 mL). The mixture is placed under stirring at 20° C. for 30 minutes; piperidine-3-carboxamide (0.390 g; 0.390 mmol) is then added, and the mixture is left under stirring at 20° C. for 16 hours. The solvent is evaporated under low pressure, and the resulting residue is treated with a 10% w/v solution of NaHCO₃ (15 mL) and extracted with CH₂Cl₂ (3×10 mL). The combined organic phases are washed with a NaCl saturated solution (15 mL), dried (Na₂SO₄), and the solvent evaporated under low pressure. The crude compound is purified by flash chromatography on silica gel column, eluting with CH₂Cl₈/EtOAc 8/2 to provide INF219 (0.110 g; yield 75%) 5 as a white solid. Mp: 60.5-61.4° C.; MS (ESI) m/z: 415-417 [M+H]⁺; ¹H-NMR (CDCl₃) δ, 7.37 (s, 1H); 7.28-7.24 (m, 3H); 7.09-6.99 (m, 5H); 6.58 (s, 1H); 5.87 (s, 1H); 3.99-3.98 (m, 31H); 3.64-3.10 (m, 2H); 2.60-2.44 (m, 1H); 1.97-1.95 (m, 1H); 1.83-1.67 (m, 1H); 1.46 (d, 2H, J=48.5 Hz); ¹³C-NMR (CDCl₃) δ, 175.1; 170.1; 135.0; 134.1; 133.8; 133.1; 130.3; 129.5; 129.4; 129.0; 128.8; 128.1; 126.7; 125.9; 48.1; 44.1; 41.7; 0.31.4; 27.2; 24.6. Cytotoxicity (MTT assay): IC₅₀>100 μM.

### Example 41—Synthesis of ethyl (Z)-3-(2-chlorophenyl)-2-((phenylsulphonyl)methyl)acrylate (INF51)

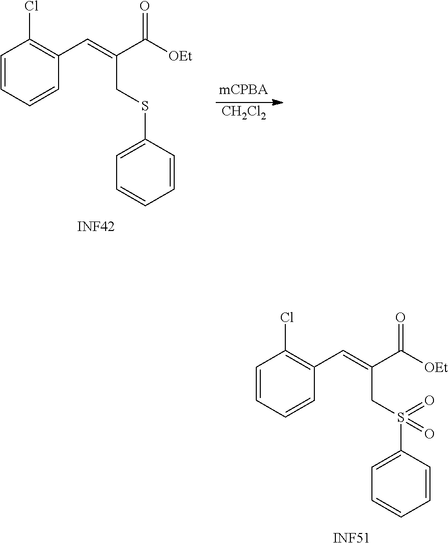 [SMILES: CCOC(=O)/C(=C/c1ccccc1Cl)CSc1ccccc1 ; "INF42" ; "CH2Cl2" ; CCOC(=O)/C(=C/c1ccccc1Cl)CS(=O)(=O)c1ccccc1 ; "INF51"]

75% mCPBA (0.301 g; 1.35 mmol) is added to a solution of INF42 (0.150 g; 0.449 mmol) in CH₂Cl₂ (10 mL), and the reaction mixture is left under magnetic stirring at 20° C. for 18 hours. The reaction mixture is extracted with a 10% w/v solution of NaOH (3×20 mL) and NaCl saturated solution (20 mL), dried (Na₂SO₄), and the solvent is removed under low pressure. The crude compound is purified by flash chromatography on silica gel column using a PE/EtOAc 8:2 mixture as eluent. Compound INF51 is obtained as a colourless oil (0.149 g; yield 91%). CI-MS (isobutane) m/z: 365-367 [M+1]⁺; ¹H-NMR (CDCl₃): δ, 8.01 (s, 1H, C═CH), 7.89-7.20 (m, 9H, ArH); 4.39 (s, 2H, CH₂SO₂); 4.09 (q, J=7.1 Hz, 2H, CH₂CH₃); 1.25 (t, J=7.1 Hz, 3H, CH₂CH₃). ¹³C-NMR (CDCl₃): δ, 166.3; 143.2; 139.7; 134.4; 134.2; 132.7; 131.0; 130.3; 130.1; 129.5; 128.8; 127.4; 123.8; 62.1; 55.2; 14.4. Cytotoxicity (MTT assay): IC₅₀ 20.1±14.3 μM.

### Example 42—Synthesis of (Z)-3-(2-chlorophenyl)-2-((phenylthio)methyl)-N-(4-sulphamoylphenethyl)acrylamide (INF220)

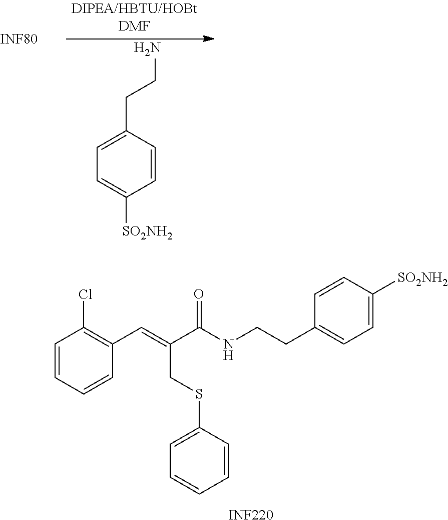

DIPEA (0.111 mL; 0.656 mmol), HOBt (0.0044 g; 0.033 mmol) and HBTU (0.187 g; 0.492 mmol) are added to a solution of TNF80 (0.100 g; 0.328 mmol) in DMF (5 mL). The mixture is placed under stirring at 20° C. for 30 minutes; 4-(2-aminoethyl)benzenesulphonamide (0.066 g; 0.328 mmol) is then added, and the mixture is left under stirring at 20° C. for 5 hours. The solvent is evaporated under low pressure, and the resulting residue is diluted with diethyl ether (20 mL) and washed with 1N HCl (2×15 mL), then with H₂O (2×15 mL). The organic phase is washed with a NaCl saturated solution (15 mL) and dried (Na₂SO₄), and the solvent is evaporated under low pressure. The crude compound is purified by flash chromatography on silica gel column, eluting with PE/EtOAc/MeOH 7/2/1 to provide INF220 (0.080 g; yield 50%) as a pale yellow semisolid. MS (ESI) m/z: 487-489 [M+H]⁺; ¹H-NMR (DMSO-D6) δ, 7.74 (d, 2H, J=8.3 Hz, ArH); 7.51 (d, 1H, J=7.9 Hz, ArH); 7.43 (d, 2H, J=8.3 Hz, ArH); 7.37 (m, 1H, ArH); 7.29-7.19 (m, 9H, ArH e CH═C); 3.89 (s, 2H, CH₂S); 3.43 (t, 2H, J=7.1 Hz, NCH₂CH₂); 2.88 (t, 2H, J=7.1 Hz, NCH₂CH₂); ¹³C-NMR (CDCl₃), 168.1; 144.4; 140.5; 134.8; 134.2; 133.9; 133.5; 133.2; 130.5; 130.1; 129.9; 129.7 (2 overlapping peaks); 129.2; 127.2; 126.8; 40.8; 35.4; 32.3. Cytotoxicity (MTT assay): IC₅₀>62.5±4.2 μM.

### Example 43—Synthesis of ethyl (Z)-1-(3-(3-chlorophenyl)-2-((phenylthio)methyl)acryloyl)piperidine-3-carboxylate (INF230)

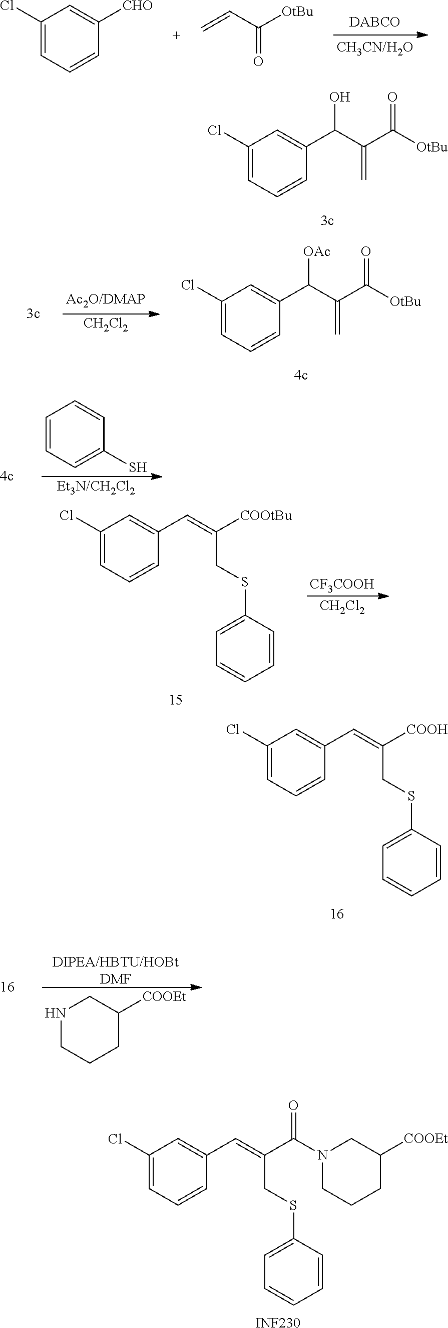

t-Butyl acrylate (15 mL; 103.3 mmol) and water (5 mL) are added to a solution of 3-chlorobenzaldehyde (4.84 g; 34.4 mmol) in CH₃CN (45 mL). DABCO (3.7 g; 34.4 mmol) is then added to the mixture, and the reaction is left under stirring for 7 days at 20° C. The mixture is diluted with CH₂Cl₂ (30 mL) and extracted with 1N HCl (3×30 mL) and NaCl saturated solution (30 mL), then dried (Na₂SO₄), and the solvent evaporated under low pressure. The residue is purified by flash chromatography on silica gel column, eluting with a PE/acetone 8/2 mixture. t-butyl 2-((3-chlorophenyl)(hydroxy)methyl)acrylate (3c) is obtained as a colourless oil (4.23 g; yield 46%).

Acetic anhydride (2.09 g; 20.41 mmol) dissolved in CH₂Cl₂ (20 mL) is added slowly over a period of 1 hour to a solution of 3c (4.23 g; 15.7 mmol) and DMAP (380 mg, 3.14 mmol) in CH₂Cl₂ (20 mL), maintaining the mixture under stirring at 20° C. The reaction mixture is extracted with water (30 mL) and 10% w/v NaHCO₃ (3×30 mL), then with a NaCl saturated solution (30 mL). The organic phase is dried (Na₂SO₄), and the solvent is evaporated under low pressure. The residue is purified by flash chromatography on silica gel using a PE/EtOAc 95/5 mixture as eluent. t-butyl 2-(acetoxy(3-chlorophenyl)methyl)acrylate (4c) is obtained as a colourless oil (3.5 g; yield: 72%).

Thiophenol (1.15 mL; 11.3 mmol) and triethylamine (1.88 mL; 13.5 mmol) are added to a solution of 4c (3.5 g; 11.3 mmol) in CH₂Cl₂ (50 mL), maintained in an inert atmosphere (N₂). The reaction is left under vigorous stirring for 30 minutes at 20° C. The reaction mixture is then diluted with H₂O (20 mL) and extracted with 1N HCl (3×40 mL) and NaCl saturated solution (30 mL). The organic phase is dried (Na₂SO₄), and the solvent is removed under low pressure. The residue is purified by flash chromatography on silica gel column, eluting with PE/DCM 8/2 and then increasing the percentage of DCM in the mixture to PE/EtOAc 6/4; t-butyl (Z)-3-(3-chlorophenyl)-2-((phenylthio)methyl)acrylate (15) is obtained; 2.9 g; yield 71%.

Compound 15 (0.828 g; 2.49 mmol) is dissolved in a mixture of TFA in 10% DCM (25 mL). The reaction mixture is placed under stirring at 20° C. for 16 hours, and then diluted with H₂O (30 mL) and extracted with DCM (3×40 mL). The organic phases are washed with a NaCl saturated solution and dried (Na₂SO₄), and the crude solid is recrystallised from acetonitrile to obtain (Z)-3-(3-chlorophenyl)-2-((phenylthio)methyl)acrylic acid (16) as a white solid (1.6 g; yield: 65%).

DIPEA (0.559 mL; 3.28 mmol), HOBt (22 mg; 0.164 mmol) and HBTU (0.933 g; 2.46 mmol) are added to a solution of 16 (0.500 g; 1.64 mmol) in DMF (10 mL), and ethyl nipecotate (0.255 mL; 1.64 mmol) is added after 30 minutes. The reaction mixture is left to react under magnetic stirring for 18 hours at 20° C. The mixture is diluted with 15 mL of diethyl ether, and the organic phase is washed with 1N HCl (3×15 mL) and NaCl saturated solution (15 mL), and dried (Na₂SO₄). The solvent is evaporated under low pressure, and the resulting residue is purified by flash chromatography on silica gel column, eluting with PE/EtOAc 8/2. Ethyl (Z)-1-(3-(3-chlorophenyl)-2-((phenylthio)methyl)acryloyl)piperidine-3-carboxylate (INF230) is obtained as a pale yellow oil (0.301 g; yield: 41%). MS (ESI) m/z: 466-468 [M+Na]⁺; ¹H-NMR (CDCl₃): δ, 7.41-7.26 (m, 2H, Ar—H); 7.25-7.22 (m, 1H, Ar—H); 7.21-7.12 (in, 5H, Ar—H); 6.49 (s, 1H, C═CH); 4.61-4.47 (m, 1H, CH-pip); 4.04 (q, J=7.1 Hz; 2H, O—CH₂); 3.93-3.83 (m, 2H, S—CH₂); 3.08-2.94 (m, 2H, N—CH₂ₚᵢₚ); 2.44 (m, 2H, CH₂ₚᵢₚ); 2.09-1.95 (m, 1H, CH₂ₚᵢₚ); 1.74-1.57 (m, 2H, CH₂ₚᵢₚ); 1.41-1.39 (m, 1H, CH₂ₚᵢₚ); 1.24 (t, J=7.1 Hz; 3H, CH₃).

### Example 44—Synthesis of ethyl (Z)-1-(3-(2-nitrophenyl)-2-((phenylthio)methyl)acryloyl)piperidine-3-carboxylate (INF231)

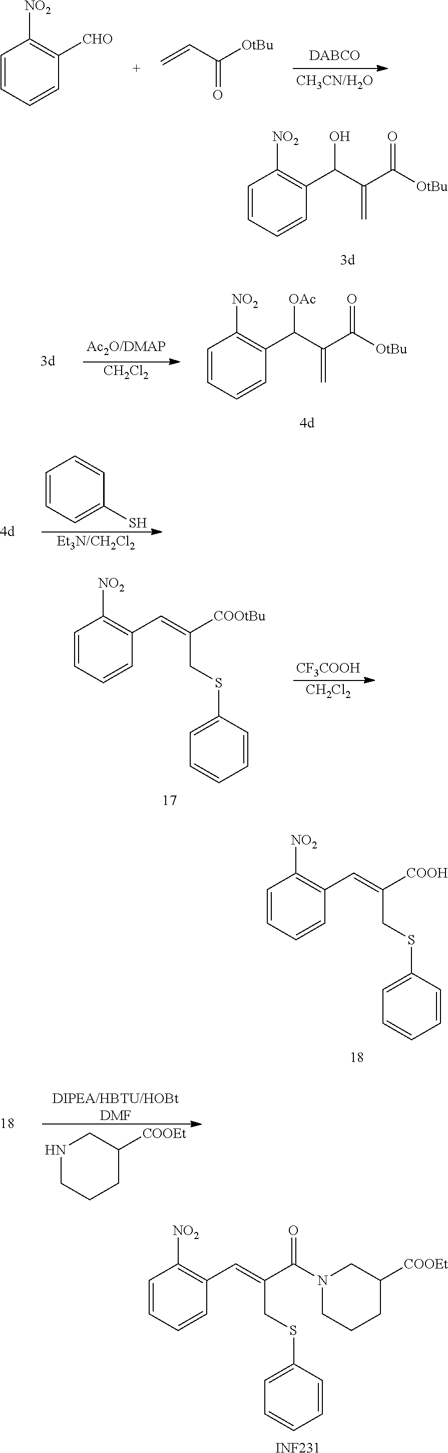

t-Butyl acrylate (9.3 mL; 84.89 mmol) and water (5 mL) are added to a solution of 2-nitrobenzaldehyde (5.20 g; 34.4 mmol) in CH₃CN (45 mL). DABCO (3.86 g; 34.4 mmol) is then added to the mixture, and the reaction is left under stirring for 4 days at 20° C. The mixture is diluted with CH₂Cl₂ (30 mL) and extracted with 1N HCl (3×40 mL) and NaCl saturated solution (30 mL), then dried (Na₂SO₄), and the solvent evaporated under low pressure. The residue is purified by flash chromatography on silica gel column, eluting with a DCM/EtOAc 9/1 mixture. t-butyl 2-((2-nitrophenyl)(hydroxy)methyl)acrylate (3d) is obtained as a colourless oil (5.76 g; yield 60%).

Acetic anhydride (2.73 g; 26.53 mmol) dissolved in CH₂Cl₂ (20 mL) is added slowly over a period of 1 hour to a solution of 3d (5.70 g; 20.41 mmol) and DMAP (0.50 mg, 4.08 mmol) in CH₂Cl₂ (70 mL) at 0° C., maintaining the mixture under stirring at 20° C. The reaction mixture is extracted with water (30 mL) and 10% w/v NaHCO₃ (3×30 mL), then with a NaCl saturated solution (30 mL). The organic phase is dried (Na₂SO₄), and the solvent is evaporated under low pressure. The residue is purified by flash chromatography on silica gel using a PE/EtOAc 9/1 mixture as eluent. t-butyl 2-(acetoxy(2-nitrophenyl)methyl)acrylate (4d) is obtained as a colourless oil (2.35 g; yield 36%).

Thiophenol (0.750 mL; 7.31 mmol) and triethylamine (1.26 mL; 8.77 mmol) are added to a solution of 4d (2.35 g; 7.31 mmol) in CH₂Cl₂ (50 mL), maintained in an inert atmosphere (N₂). The reaction is left under vigorous stirring for 30 minutes at 20° C. The reaction mixture is then diluted with H₂O (30 mL) and extracted with 1N HCl (3×40 mL) and NaCl saturated solution (20 mL). The organic phase is dried (Na₂SO₄), and the solvent is removed under low pressure. The residue is purified by flash chromatography on silica gel column, eluting with a PE/EtOAc 9/1 mixture to provide t-butyl (Z)-3-(2-nitrophenyl)-2-((phenylthio)methyl)acrylate (17) as a pale yellow oil (1.4 g; yield: 52%).

Compound 17 (0.828 g; 2.49 mmol) is dissolved in a mixture of TFA in 10% DCM (25 mL). The reaction mixture is placed under stirring at 20° C. for 16 hours, and then diluted with H₂O (30 mL) and extracted with dichloromethane (3×40 mL). The organic phases are washed with a NaCl saturated solution, dried (Na₂SO₄), and evaporated under low pressure to obtain (Z)-3-(2-nitrophenyl)-2-((phenylthio)methyl)acrylic acid (18) as a cream-coloured solid (0.944 g; yield 79%).

DIPEA (0.510 mL; 1.98 mmol), HOBt (18 mg; 0.15 mmol) and HBTU (0.850 g; 2.24 mmol) are added to a solution of 18 (0.472 g; 1.49 mmol) in DMF (10 mL), and ethyl nipecotate (0.231 mL; 01.49 mmol) is added after 30 minutes. The reaction mixture is left to react under magnetic stirring for 18 hours at 20° C. The mixture is diluted with 15 mL of diethyl ether, and the organic phase is washed with 1N HCl (3×15 mL) and NaCl saturated solution (15 mL), and dried (Na₂SO₄). The solvent is evaporated under low pressure, and the resulting residue is purified by flash chromatography on silica gel column, eluting with PE/EtOAc 6/4 followed by PE/EtOAc 1/1. Ethyl (Z)-1-(3-(2-nitrophenyl)-2-((phenylthio)methyl)acryloyl)piperidine-3-carboxylate (INF231) is obtained as a pale yellow oil (0.316 g; yield: 47%). MS (ESI) m/z: 477 [M+Na]⁺; ¹H-NMR (CDCl₃): δ, 8.17-8.12 (m, 1H, Ar—H); 7.65-7.41 (m, 3H, Ar—H); 7.08-7.01 (m, 5H, Ar—H); 6.82 (s, 1H, C═CH); 4.59-4.42 (m, 1H, CH-pip); 4.18-4.05 (m; 2H, O—CH₂); 3.91-3.86 (m, 2H, S—CH₂); 3.21-2.93 (m, 2H, N—CH₂ₚᵢₚ); 2.53-2.44 (m, 2H, CH₂ₚᵢₚ); 1.98-1.92 (in, 1H, CH₂ₚᵢₚ); 1.67-1.55 (m, 2H, CH₂ₚᵢₚ); 1.43-1.37 (m, 1H, CH₂ₚᵢₚ); 1.25 (t, J=7.2 Hz; 3H, CH₃).

### Example 45—Stability

The compound INF177 was incubated at the concentration of 100 μM in PBS pH 7.4 (0.1% DMSO) at 37° C., in the absence and in the presence of excess glutathione or cysteamine (10×). The reaction mixture underwent repeated HPLC analyses over 24 hours.

The HPLC analysis was conducted with an HP 1200 chromatography system (Agilent Technologies, Palo Alto, CA, USA) consisting of an integrated quaternary pump (model G1311A), degasser (model G1322A), UV MWD detector (model G1365D) and fluorescence detector (model G1321A). The data were processed with the HP ChemStation system program (Agilent Technologies).

The analyses were conducted using the ZORBAX SB-Phenyl column (250×4.6 mm, 5 μm; Agilent) as stationary phase and CH₃CN/H₂O (+0.1% HCOOH), 70/30 (v/v), flow rate=1.0 mL/min, as mobile phase, injecting 20 μL of sample (Rheodyne, Cotati, CA); the chromatograms were acquired at the wavelengths of 234 and 250 nm.

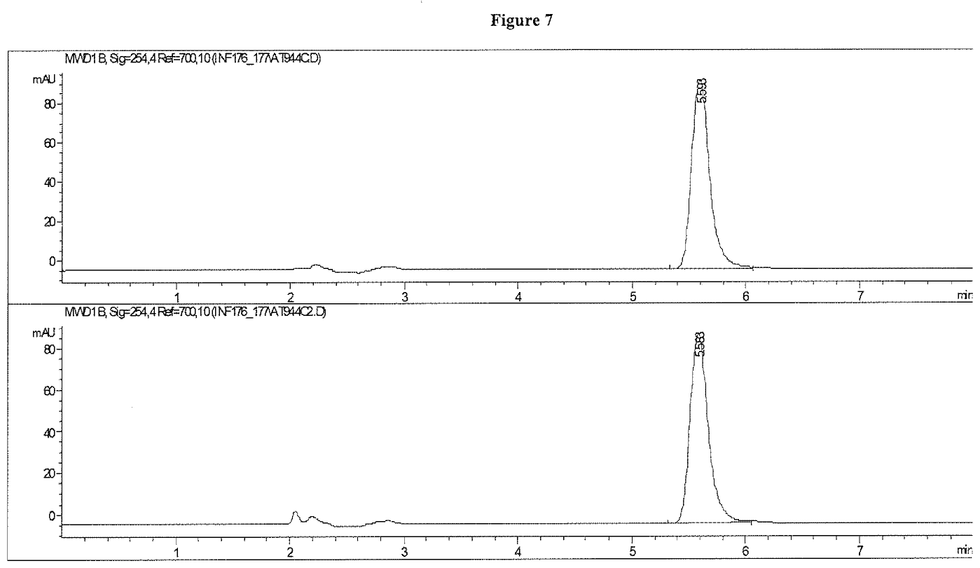

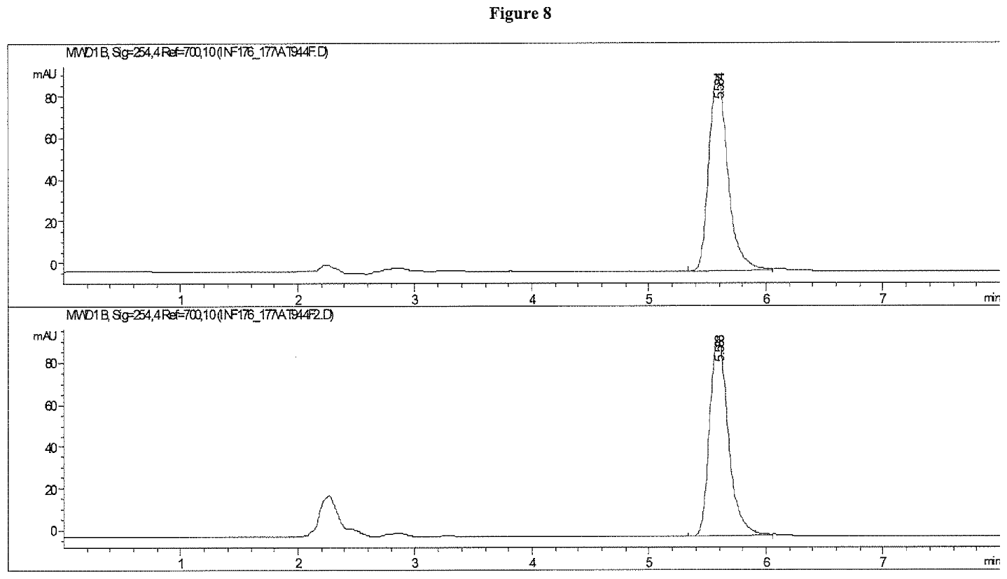

As demonstrated by the chromatograms shown in FIGS. 7 and 8, no appreciable reactivity was observed in either condition:
% compound >98% (after 4 hours with glutathione and after 2.5 hours with cysteamine),
the % compound does not fall below 96%, even when the time is increased to 24 hours.

### Bioassays

### Materials and Methods—Preparation and Treatment of Cells

THP-1 cells, a monocytic human cell line derived from the peripheral blood of a male patient suffering from acute monocytic leukaemia (www.atcc.org), were cultured in RPMI 1640 medium (Aurogene, Rome, Italy), with the addition of foetal bovine serum (10%, Aurogene), L-glutamine (2 mM, Aurogene), penicillin (100 IU/ml, Aurogene) and streptomycin (100 mg/ml, Aurogene). The medium was changed every 2-3 days, and the cells were maintained in an incubator at 37° C., with 5% CO₂, and with suitable humidity.

The cells were seeded in 48-well plates (90.000 cells/well), and differentiated with phorbol 12-myristate 13-acetate (PMA—50 nM, 24 hours; Sigma-Aldrich). On the next day, the differentiated THP-1s were washed twice with the balanced saline solution phosphate-buffered saline (PBS), and stimulated with lipopolysaccharide (LPS) (10 μg/mL, 4 hours; Sigma-Aldrich), prepared in serum-free medium. After 4 hours the cells were incubated with the compounds at the concentration of 10 μM for 1 hour, operating in triplicate; INF176 and INF177 were tested at three different concentrations: 1, 10 and 20 nM. Finally, the cells were stimulated with ATP 5 mm for 1.5 hours. The supernatants were then harvested for the subsequent analyses.

### Example 46—Lactate Dehydrogenase (LDH) Release

LDH release into the supernatant obtained as described above was quantified using the CytoTox 96 nonradioactive cytotoxicity assay (Promega Corporation, Madison, MI, USA), a colorimetric assay wherein LDH activity is measured with an NADH-dependent enzymatic reaction:

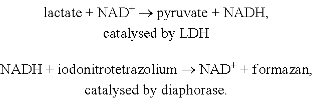

Briefly, an equal volume of CytoTox Reagent was added to the supernatant and, after 30 minutes' incubation, the reaction was stopped by adding the stop solution. The formazan concentration was determined by measuring absorbance, using a microplate reader (Victor X4—EnSight, PerkinElmer, Waltham, MA, USA), at λ=490 nm. Cell death was expressed according to the manufacturer's instructions.

### Example 47—IL-1β Release

IL-1β release into the supernatant of cells treated as described above was determined with the Human IL-1 beta Uncoated ELISA kit (Invitrogen, Waltham, MA, USA), according to the manufacturer's instructions.

Briefly, on the first day the 96-well plate (Nunc Immuno plate, Thermofisher) was coated with anti-IL-1β capture antibody, included in the kit, and left to incubate for 16 hours at 4° C., under stirring. On the next day, after washing the wells with PBS buffer+0.05% Tween-20 and incubating with a saturated solution for 1 hour at room temperature, the standard protein or samples were added to each well, and the plate was incubated overnight at 4° C., under stirring. The next day, after suitable washes with PBS+0.05% Tween-20, the anti-IL-1β biotinylate secondary antibody, included in the kit, was added; after one hour, the plate was washed, and avidin conjugated with the enzyme horseradish peroxidase (HRP) for 30 minutes at room temperature was added, followed by the substrate tetramethylbenzidine (TMB) for 15 minutes. The reaction was stopped with a 2N solution of H₂SO₄.

The interleukin levels were determined by measuring absorbance at X=450 nm, using a microplate reader (Victor X4—EnSight).

### Example 48—Cytotoxicity (MTT Assay)

The THP-1 cells were seeded in a 96-well plate (15.000 cells/well) and incubated with the test compounds at four different concentrations (0.1, 1, 10 and 100 μM), operating in triplicate. The cells were placed in an incubator at 37° C., with 5% CO₂, and cell viability was measured after 72 hours' incubation, using the MTT assay. This is a colorimetric assay, based on conversion of water-soluble 3-(4,5-dimethylthiazol-2-yl)-2,5-diphenyltetrazolium bromide (MTT; Sigma-Aldrich) to a violet-coloured insoluble formazan, by the mitochondrial dehydrogenase present in the live cells. The formazan is solubilised in acidified isopropanol, and the concentration is determined by measuring absorbance with a microplate reader (Victor X4—Ensight, PerkinElmer) at λ=570 nm. Viability was expressed as a percentage of the viability of the control cells, which were either untreated or treated with the carrier. The IC₅₀ was calculated with the Graph Pad Prism software.

### Example 49—Anti-Pyroptotic Activity and Inhibition of IL-1Beta Release

The compound INF176 was tested to measure its ability to inhibit NLRP3-dependent cell pyroptosis in human macrophages, using the experimental protocol previously published [Cocco et al. 2017].

The THP-1 cells were differentiated into macrophages by treating them with PMA 50 nM (24 hours) and then with LPS (10 μg/mL) for 4 hours. The cells were treated with the compound (10 μM) for 1 hour. Pyroptosis was induced by treatment with ATP 5 mm. After 90 minutes, cell death was evaluated by measuring the LDH level in the cell supernatant using the CytoTox 96 Non-Radioactive Cytotoxicity Assay (Promega Corporation, Madison, MI, USA).

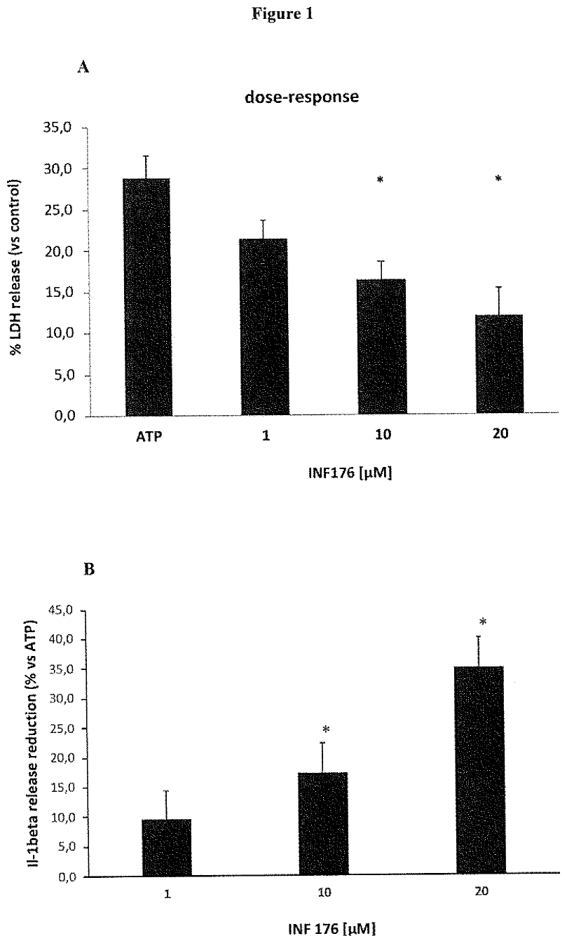

Compound INF176 is able to inhibit NLRP3-dependent cell pyroptosis induced by LPS/ATP in a dose-dependent manner, with inhibition of 25.7±5.9-58.7±7.6% in the range of concentrations tested (FIG. 1A). INF176 also inhibits IL-1beta release from human macrophages stimulated with pro-inflammatory substances such as LPS/ATP. Said effect is also dose-dependent, with inhibition of 35±1.2% at the maximum concentration tested (FIG. 1B).

### Example 50—Anti-Inflammatory Activity in Colitis

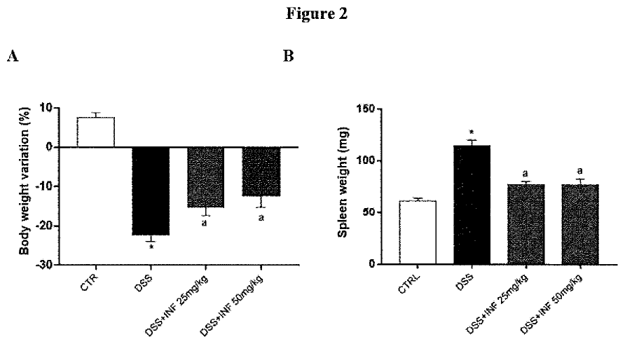

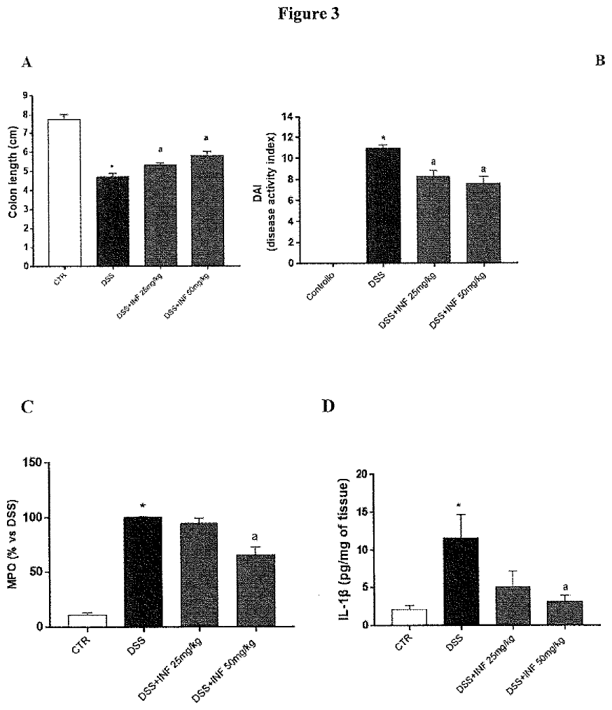

Studies conducted in vivo on an experimental murine model of colitis induced by dextran sodium sulphate (DSS) demonstrated a good level of efficacy of INF176 in counteracting intestinal inflammation. In particular, oral administration (p.o.) of INF176 at the doses of 25 mg/kg/day and 50 mg/kg/day gave rise to an improvement in the systemic and tissue parameters associated with colitis (FIGS. 2-3). In particular, INF176 counteracts weight loss and increased spleen weight in the animal, and significantly reduces the disease activity index (DAI), the levels of interleukin-1beta and myeloperoxidase (index of degree of infiltration of inflammatory cells) in colon tissues (FIGS. 2-3).

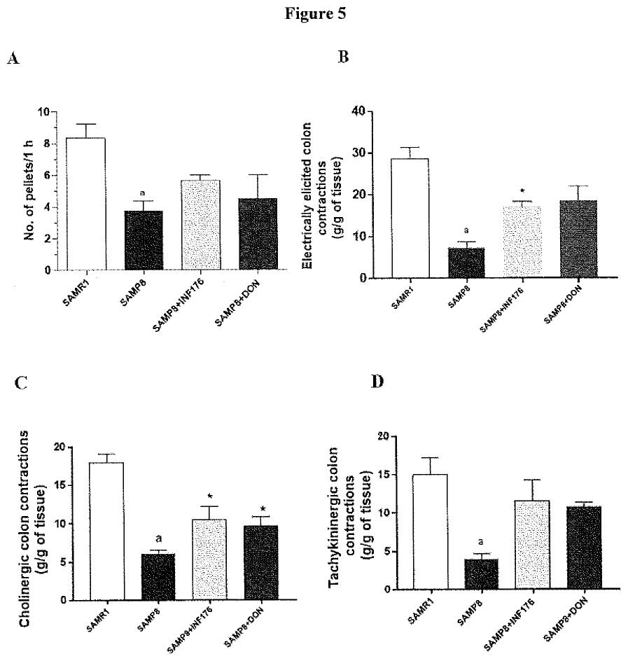

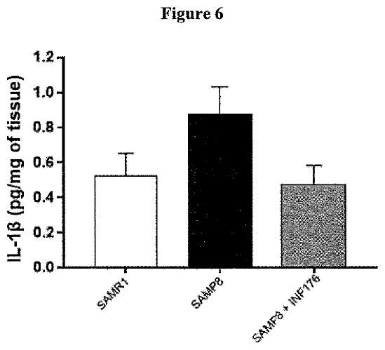

It should be noted that administration of INF176 improves slowing of colon transit in SAMP8 animals (FIG. 5A). Moreover, in vitro studies demonstrate that INF176 significantly improves the colon contractions elicited by electrical stimuli, significantly enhancing both cholinergic and tachykininergic colon contractions (FIGS. 5B, 5C and 5D). Treatment of SAMP8 animals with INF176 also reduces the increase in IL-1β levels in the colon (FIG. 6).

### Example 51—Activity in the Treatment of Neurodegenerative Disorders

Compound INF176 was tested in vivo in an animal model of mice with spontaneous accelerated senescence (SAMP8), used as a model of neurodegenerative disorders such as mild cognitive impairment (MCI), which in most cases evolve to Alzheimer's disease (AD).

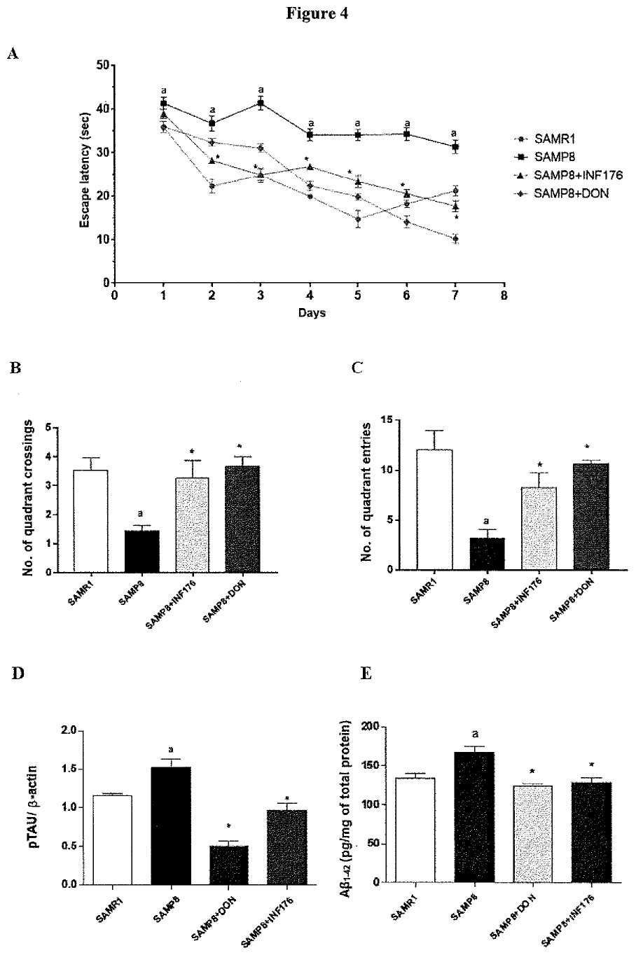

Chronic administration (2 months) of compound INF176 (50 mg/kg/day p.o.) to SAMP8 animals, starting in the earliest stages of the disease, before the appearance of the first symptoms, significantly counteracts cognitive decline (evaluated with the Morris test and expressed as escape latency, number of crossings in the quadrant, and number of entries into the quadrant) and significantly reduces expression of p-tau protein (considered to be a disease marker) in brain tissues, with results comparable to donepezil (DON), a medicament currently approved and used for the treatment of AD (FIGS. 4A, 4B and 4C).

### Example 52—Molecular Docking Studies

The induced-fit docking (IFD) [Sherman et al.] protocol included in the Maestro software suite (release 2022-1, Schröedinger Inc.) was used for the molecular docking studies. The structure of NLRP3 was obtained from the Protein Data Bank (PDB ID:7ALV), and processed in Maestro. The hydrogen atoms were added with PropKa 3.0, assuming a pH of 7.4. The residue of Ala228 was selected as centre of the docking cavity, and the radius of the cavity was set to 20 Å. In the first step of the IFD protocol, 10 initial poses were used with softened-potential docking, and subsequently refined with the Prime package, to accommodate the ligand by reorienting the side chains of the residues around the ligand. Ligands characterised within 30 Kcal/mol of the minimum energy structure were processed with a last round of docking and scoring with Glide. The ligands were then again subjected to docking in the binding site, and the score was allocated with the Extra Precision (XP) scoring function in Glide [Friesner et al.]. The results of the studies conducted are set out in Table 3:

TABLE 3

| Compound | DOCKING SCORE | XP GScore | glide gscore | glide emodel |
|---|---|---|---|---|
| 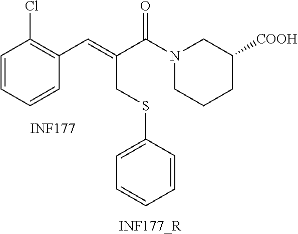 | −8.54 | −8.54 | −8.54 | −85.34 |
| Br1 | −8.83 | −8.83 | −8.83 | −54.61 |
| Br2S | −12.19 | −12.19 | −12.19 | −77.79 |
| Br2R | −9.30 | −9.30 | −9.30 | −79.98 |
| Br4R | −8.79 | −8.79 | −8.79 | −67.23 |
| Br7 | −8.76 | −8.76 | −8.76 | −56.36 |
| Br8R | −7.42 | −7.42 | −7.42 | −73.78 |
| Br9R | −10.11 | −10.11 | −10.11 | −83.40 |
| Br10R | −11.45 | −11.45 | −11.45 | −74.10 |
| Br10S | −11.19 | −11.19 | −11.19 | −65.00 |
| Br11R | −8.79 | −8.79 | −8.79 | −82.68 |
| Br12 | −8.63 | −8.63 | −8.63 | −67.34 |
| INF80 | −9.21 | −9.21 | −9.21 | −55.21 |
| Br13 | −8.13 | −8.13 | −8.13 | −65.27 |
| Br14R | −8.48 | −8.48 | −8.48 | −76.94 |
| Br14S | −8.78 | −8.78 | −8.78 | −72.19 |
| Br15 | −7.17 | −7.17 | −7.17 | −51.13 |
| Br16R | −9.73 | −9.73 | −9.73 | −77.59 |
| Br17 | −9.24 | −9.24 | −9.24 | −63.13 |
| Br18R | −9.86 | −9.86 | −9.86 | −86.47 |
| Br4S | −10.70 | −10.70 | −10.70 | −66.78 |
| Br8S | −8.89 | −8.89 | −8.89 | −79.95 |
| Br11S | −9.95 | −9.95 | −9.95 | −92.30 |

The studies demonstrate that INF177, metabolite of INF176, which proved active in the tests conducted according to examples 49, 50 and 51 reported above, is characterised by a docking score of −8.54, representative of the bonding capacity of the NLRP3 protein in the NACHT domain (PDB ID:7ALV). The compounds exemplified in the table have docking scores equivalent to those of INF177, which are therefore predictive of the efficacy of said compounds.

### REFERENCES

Basiorka, A. A. et al. (2016). The NLRP3 inflammasome functions as a driver of the myelodysplastic syndrome phenotype. Blood Vol 128, 2960-2975.
Bennett, J. M. et al. (2018). Inflammation-nature's way to efficiently respond to all types of challenges: implications for understanding and managing “the epidemic” of chronic diseases. Front. Med. 5, 316.
Bertinaria M. et al. (2019). Development of covalent NLRP3 inflammasome inhibitors: Chemistry and biological activity. Arch. Biochem. Biophys. Vol 670, 116-139.
Boaru S. G. et al. (2012) Expression analysis of inflammasomes in experimental models of inflammatory and fibrotic liver disease. J. Inflamm. (Lond.) Vol 9, 49.
Booshehri M L, and Hoffman H M. (2019). CAPS and NLRP3. J Clin. Immunol. Vol 39, 277-286.
Freeman T. L. and Swartz, T. H. (2020). Targeting the NLRP3 Inflammasome in severe COVID-19. Front. Immunol. Vol 11, Art. 1518.
Chauhan D et al. (2020). Therapeutic modulation of inflammasome pathways. Immunol. Rev. pp. 1-16.
Cocco M et al. (2017). Development of an acrylate derivative targeting the NLRP3 inflammasome for the treatment of Inflammatory Bowel Disease. J Med. Chem. Vol 60, 3656-3671.
Cornelius D. C. et al. (2020). NLRP3 inflammasome inhibition attenuates sepsis-induced platelet activation and prevents multi-organ injury in cecal-ligation puncture. PLOSone Vol 15, e0234039.
Furman D. et al. (2019) Chronic inflammation in the etiology of disease. Nat. Med. Vol 25, 1822-1832.
Fusco R et al. (2020). Focus on the role of NLRP3 inflammasome in Diseases. Int. J. Mol. Sci. Vol 21, 4223.
GBD 2017 Causes of Death Collaborators. (2018) Global, regional, and national age-sex-specific mortality for 282 causes of death in 195 countries and territories, 1980-2017: a systematic analysis for the Global Burden of Disease Study 2017. Lancet Vol 392, 1736-1788.
Groslambert M and Py B F (2018). Spotlight on the NLRP3 inflammasome pathway. J. Inflammation Res. Vol. 11, 359-374.
Guo C. et al. (2018). NLRP3 inflammasome activation contributes to the pathogenesis of rheumatoid arthritis. Clin. Exp. Immunol. Vol 194, 231-243.
He Y. et al. (2016). Mechanism and Regulation of NLRP3 Inflammasome Activation. Trends Biochem. Sci. Vol 41, 1012-1021.
Heneka M. T. et al. (2018). Inflammasome signalling in brain function and neurodegenerative disease. Nat. Rev. Neurosci. Vol 19, 610-621.
Jing Y and Fu J (2019) Novel Insights Into the NLRP3 Inflammasome in Atherosclerosis. J. Am. Heart Ass. Vol 8, e012219.
Kluck V et al. (2020). Dapansutrile, an oral selective NLRP3 inflammasome inhibitor, for treatment of gout flares: an open-label, dose-adaptive, proof-of-concept, phase 2a trial. Lancet Rheumatology. Vol 2, e270-e280.
Lee, H M et al. (2013) Upregulated NLRP3 inflammasome activation in patients with type 2 diabetes. Diabetes Vol 62, 194-204.
Li Z. et al (2020). Role of NLRP3 inflammasome in autoimmune diseases. Biomedicine & Pharmacotherapy Vol 130, 110542
Liu L et al. (2017). The Pathogenic Role of NLRP3 Inflammasome Activation in Inflammatory Bowel Diseases of Both Mice and Humans. J. Crohn's Colitis Vol 11, 737-750.
Malhotra S. et al. (2020). NLRP3 inflammasome as prognostic factor and therapeutic target in primary progressive multiple sclerosis patients. Brain Vol 143. 1414-1430.
Mangan et al. (2018). Targeting the NLRP3 inflammasome in inflammatory diseases. Nat. Rev. Drug Dis. Vol. 17, 588-606.
Martinon F. et al. (2006). Gout-associated uric acid crystals activate the NALP3 inflammasome, Nature Vol 440, 237-241.
Mridha A. R. et al (2017). NLRP3 inflammasome blockade reduces liver inflammation and fibrosis in experimental NASH in mice. J. Hepatol. Vol 66, 1037-1046.
Mortimer L et al. (2016). NLRP3 inflammasome inhibition is disrupted in a group of auto-inflammatory disease CAPS mutations. Nat. Immunol. Vol 17, 1176-1188.
Netea, M. G. et al. (2017) A guiding map for inflammation. Nat. Immunol. 18, 826-831.
Primiano M. J. et al. (2016). Efficacy and pharmacology of the NLRP3 inflammasome inhibitor CP-456,773 (CRID3) in murine models of dermal and pulmonary inflammation. J. Immunol. Vol 197, 2421-2433.
Ratajczak M. Z. et al. (2020). The Nlrp3 inflammasome as a “rising star” in studies of normal and malignant hematopoiesis. Leukemia vol 34, 1512-1523.
Ren, H. et al. (2018). Selective NLRP3 (pyrin domain-containing protein 3) inflammasome inhibitor reduces brain injury after intracerebral hemorrhage. Stroke Vol 49, 184-192.
Shimizu H. et al. (2019). Pro-inflammatory role of NLRP3 inflammasome in experimental sterile corneal inflammation. Sci. Rep. Vol 9, 9596.
Slavich, G. M. (2015) Understanding inflammation, its regulation, and relevance for health: a top scientific and public priority. Brain Behav. Immun. 45, 13-14.
Straub R H and Schradin C. (2016) Chronic inflammatory systemic diseases: An evolutionary trade-off between acutely beneficial but chronically harmful programs. Evolution, Medicine, and Public Health pp. 37-51.
Szekanecz Z. et al. (2019). The NLRP3 inflammasome—interleukin 1 pathway as a therapeutic target in gout. Arch. Biochem Biophys. Vol 670, 82-93.
Theofani E. et al. 2019. Targeting NLRP3 Inflammasome Activation in Severe Asthma. J. Clin. Med. Vol. 8, 1615.
Toldo, S and Abbate, A. (2018). The NLRP3 inflammasome in acute myocardial infarction. Nat. Rev. Cardiol. Vol 15, 203-214.
Vandanmagsar B et al. (2011) The NLRP3 inflammasome instigates obesity-induced inflammation and insulin resistance. Nat. Med. Vol 17, 179-188.
Vande Walle L et al. (2014). Negative regulation of the NLRP3 inflammasome by A20 protects against arthritis. Nature Vol 512, 69-73.
Wang L et al. (2014) NLRP3 and downstream cytokine expression elevated in the monocytes of patients with coronary artery disease. Arch. Med. Sci. Vol 10, 791-800.
Wu X et al. (2017). Relevance of the NLRP3 inflammasome in the Pathogenesis of Chronic Liver Disease. Front. Immunol. Vol 8, Art 1728.
Yin Z et al. (2014). Transcriptome analysis of human adipocytes implicates the NOD-like receptor pathway in obesity-induced adipose inflammation. Mol. Cell. Endocrinol. Vol 394, 80-87.
Youm Y-H et al. (2013). Canonical Nlrp3 inflammasome links systemic low-grade inflammation to functional decline in aging. Cell. Metab. VOL 18, 519-532.
Zahid A et al. (2019). Pharmacological Inhibitors of the NLRP3 Inflammasome. Front. Pharmacol. Vol 10, Art. 2538.
Zhen Y and Zhang H (2019). NLRP3 inflammasome and Inflammatory Bowel Disease. Front. Immunol. Vol 10, Art 276.
Sherman, Woody, Hege S. Beard, and Ramy Farid. “Use of an induced fit receptor structure in virtual screening.” Chemical biology & drug design 67.1 (2006): 83-84.
Friesner, Richard A., et al. “Glide: a new approach for rapid, accurate docking and scoring. 1. Method and assessment of docking accuracy.” Journal of medicinal chemistry 47.7 (2004): 1739-1749.

1. Method of preventing and/or treating diseases and/or disorders mediated by the NLRP3 inflammasome in an individual need thereof with compounds of general formula (I): 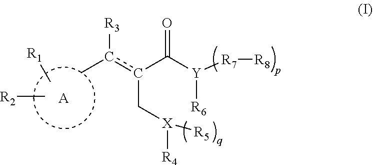
wherein
A is a C₃-C₁₀-cycloalkyl, preferably monocyclic or bicyclic C₅-C₁₀-cycloalkyl; 5- to 10-membered, monocyclic or bicyclic, saturated or partly saturated heterocycle; monocyclic or bicyclic C₆-C₁₀-aryl; 5- to 10-membered monocyclic or bicyclic heteroaryl; A is preferably a 5 or 6-membered, saturated or partly saturated, monocyclic heterocycle, or a 9 or 10-membered, saturated or partly saturated, bicyclic heterocycle; or a monocyclic C₅-C₆-aryl, or a bicyclic C₉-C₁₀-aryl; or a 5 or 6-membered monocyclic heteroaryl or a 9 or 10-membered bicyclic heteroaryl; wherein the heteroatom is preferably N or O; more preferably A is phenyl, naphthyl, furanyl or indolyl, and most preferably A is phenyl;
R₁ and R₂, which are the same or different, can occupy any position on A, and can be hydrogen; halogen selected from F, Cl, Br or I; linear or branched, substituted or unsubstituted, saturated or unsaturated C₁-C₄-alkyl; linear or branched, substituted or unsubstituted, saturated or unsaturated C₁-C₄-alkoxy; a nitro group; nitrile; a substituted or unsubstituted amido group; a substituted or unsubstituted amino group; a substituted or unsubstituted ester group; a trifluoromethyl group; R₁ and R₂ are preferably hydrogen, halogen such as F, Cl, Br or I, linear or branched C₁-C₄-alkyl, linear or branched C₁-C₄-alkoxy, a nitro group; R₁ and R₂ are more preferably hydrogen, chloro, bromo, methyl, methoxy or a nitro group; most preferably R₁ is hydrogen and R₂ is chloro; R₁ or R₂ is preferably in the 2 position when A is phenyl; [symbol]
is a single bond or a double bond;
R₃ is selected from —H, —OH, —OR₉ and —O(CO)R₉, wherein R₉ can be hydrogen, a linear or branched, substituted or unsubstituted, saturated or unsaturated C₁-C₄-alkyl; R₃ is preferably hydrogen or —OH; R₃ is more preferably hydrogen;
in the 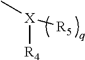
group X is selected from N, O, S, S(O) and SO₂;
R₄ can be a linear or branched, substituted or unsubstituted, saturated or unsaturated C₁₋₄ alkyl group; monocyclic or bicyclic C₃-C₁₀-cycloalkyl; substituted or unsubstituted, preferably a C₃-C₆-cycloalkyl; monocyclic or bicyclic C₆-C₁₄-aryl, substituted or unsubstituted, preferably a C₆-C₁₀-aryl, more preferably a C₅-C₆-aryl; 5- to 10-membered heterocycle, saturated or partly saturated, monocyclic or bicyclic, substituted or unsubstituted, preferably a C₅-C₆-heterocycle; monocyclic or polycyclic 5- to 14-membered heteroaryl, preferably monocyclic or bicyclic, substituted or unsubstituted, preferably a C₅-C₆-heteroaryl; R₄ is preferably monocyclic or bicyclic C₆-C₁₀-aryl, substituted or unsubstituted, or C₃-C₆-cycloalkyl, substituted or unsubstituted; more preferably, R₄ is cyclohexyl or phenyl;
q is 0 (zero) or 1; when q is equal to 1, X is N and R₅ is hydrogen; a linear or branched, substituted or unsubstituted, saturated or unsaturated C₁-C₄-alkyl group; monocyclic or bicyclic C₃-C₁₀-cycloalkyl;
alternatively, the 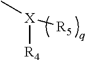
group can be an amino-acid residue wherein:
X is an N, S or O atom of the side chain of an amino acid, preferably natural, selected from serine; tyrosine; threonine; lysine; cysteine; q is zero and R₄ is the remainder of the amino acid optionally protected on the NH₂ and/or COOH terminal groups; the terminal NH₂ group is preferably acetylated; the amino-acid residue is preferably N-acetylcysteine or N-Boc cysteine methyl ester; or
X is the N atom of the terminal amino group bonded to the stereogenic carbon atom in alpha of a preferably natural, optionally protected amino acid, selected from alanine, arginine, asparagine, aspartic acid, cysteine, glycine, glutamic acid, glutamine, histidine, isoleucine, leucine, lysine, methionine, phenylalanine, proline, serine, threonine, tryptophan, tyrosine and valine; q is equal to 1, R₅ is hydrogen; and R₄ represents the remainder of the amino-acid structure, optionally protected, for example acetylated on the N atom of the side chain or esterified with a linear or branched C₁-C₄-alkyl group, preferably methyl, on the terminal carboxyl group;
alternatively when, in the 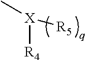
group, X is N, R₄ and R₅ are joined to form a saturated, partly saturated or unsaturated, monocyclic or bicyclic C₃-C₁₀-heterocyclic ring with the N atom; R₄ and R₅ preferably form a monocyclic C₃-C₆-heterocyclic ring with the N atom; more-preferably, R₄ and R₅ form a piperidine or pyrrolidine ring with the N atom; most preferably, R₄ and R₅ form a pyrrolidine ring with the N atom;
Y is selected from O, N and S; is preferably O or N; and is more preferably N;
when Y is an oxygen or sulfur atom, in the —(R₇-R₈)p group p is equal to zero and R₆ is selected from hydrogen, a linear or branched, substituted or unsubstituted, saturated or unsaturated C₁-C₈-alkyl group; a monocyclic or bicyclic C₃-C₁₀-cycloalkyl; a substituted or unsubstituted arylalkyl; a 6- to 14-membered monocyclic or bicyclic heteroaryl; R₆ is preferably hydrogen or a linear or branched, substituted or unsubstituted, saturated or unsaturated C₁-C₄-alkyl group; R₆ is more preferably a linear or branched, saturated, unsubstituted C₁-C₄-alkyl group; R₆ is most preferably ethyl;
when Y is a nitrogen atom, p is equal to 1, R₆ and R₇, which are the same or different, are selected from hydrogen, a linear or branched, saturated or unsaturated, substituted or unsubstituted C₁-C₄-alkyl group; a substituted or unsubstituted aryl, arylalkyl or heteroaryl group; and are preferably a substituted phenylalkyl group; more preferably —(CH₂)₂-phenyl-SO₂NH₂; most preferably R₆ is hydrogen and R₇ is —(CH₂)₂-phenyl-SO₂NH₂;
R₈ is selected from H, COOH, COOR₉, C(O)R₉, CN, CONH(R₉), S(O)NHR₉ and S(O)₂NHR₉, wherein R₉ is as defined above;
alternatively, R₆ and R₇ are joined to form a 3- to 8-membered heterocyclic ring;
R₈ is as defined above;
and their enantiomers, diastereomers, rotamers or mixtures thereof;
and the pharmaceutically acceptable salts or solvates thereof,
said method comprising administering a therapeutically effective amount of said compound to said individual.

2. The method according to claim 1, having general formula (Ia): 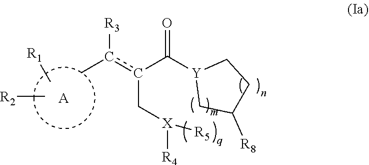
wherein
A, R₁, R₂, R₃, R₄, R₅, R₈, q, X and Y are as defined in claim 1,
n and m, which are the same or different, are 0 (zero) or an integer between 1 and 3; preferably m is 2 and n is 1; more preferably n is 2 and m is 1 or 2; most preferably, n is 2 and m is 1;
preferably, when Y is N, R₆ and R₇ are joined to form a 3- to 6-membered monocyclic substituted heterocyclic ring with the N atom; more preferably, R₆ and R₇ form a substituted piperidine or pyrrolidine ring with the N atom; most preferably, R₆ and R₇ form, with the N atom, a piperidine ring substituted in the 3 or 4 position;
and their enantiomers, diastereomers, rotamers or mixtures thereof;
and the pharmaceutically acceptable salts or solvates thereof.

3. The method according to claim 1, having general formula (Ib): 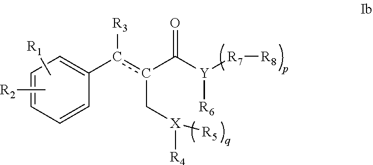
wherein
R₁, R₂, R₃, R₄, R₅, R₆, R₇, R₈, q, p, X and Y are as defined in claim 1,
and their enantiomers, diastereomers, rotamers or mixtures thereof;
and the pharmaceutically acceptable salts or solvates thereof.

4. The method according to claim 1, having general formula (Ic): 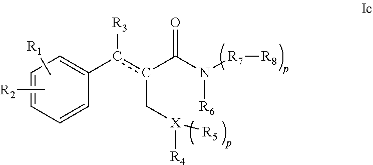
wherein
R₁, R₂, R₃, R₄, R₅, R₆, R₇, R₈, q, p and X are as defined in claim 1,
and their enantiomers, diastereomers, rotamers or mixtures thereof;
and the pharmaceutically acceptable salts or solvates thereof.

5. The method according to claim 1, wherein the compound is selected from:

| Compound | Structure |
|---|---|
| INF38s | 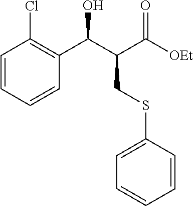 [SMILES: CCOC(=O)[C@@H](CSc1ccccc1)[C@H](O)c1ccccc1Cl] |
| | 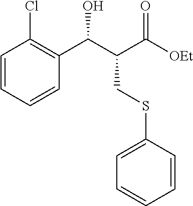 [SMILES: CCOC(=O)[C@H](CSc1ccccc1)[C@@H](O)c1ccccc1Cl] |
| INF38a | 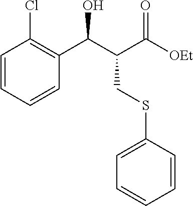 [SMILES: CCOC(=O)[C@H](CSc1ccccc1)[C@H](O)c1ccccc1Cl] |
| | 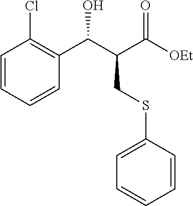 [SMILES: CCOC(=O)[C@@H](CSc1ccccc1)[C@@H](O)c1ccccc1Cl] |
| INF44 | 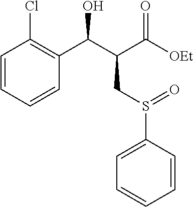 [SMILES: CCOC(=O)[C@@H](CS(=O)c1ccccc1)[C@H](O)c1ccccc1Cl] |
| | 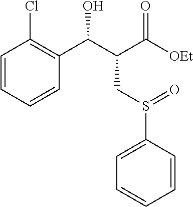 [SMILES: CCOC(=O)[C@H](CS(=O)c1ccccc1)[C@@H](O)c1ccccc1Cl] |
| INF45 | 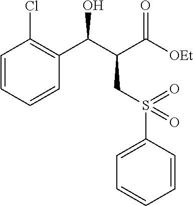 [SMILES: CCOC(=O)[C@@H](CS(=O)(=O)c1ccccc1)[C@H](O)c1ccccc1Cl] |
| | 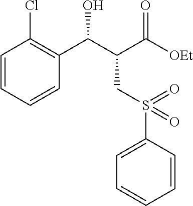 [SMILES: CCOC(=O)[C@H](CS(=O)(=O)c1ccccc1)[C@@H](O)c1ccccc1Cl] |
| INF42 | 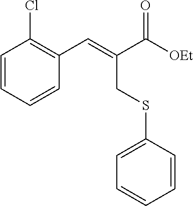 [SMILES: CCOC(=O)/C(=C/c1ccccc1Cl)CSc1ccccc1] |
| INF50 | 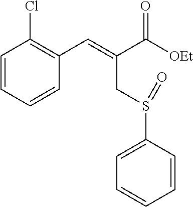 [SMILES: CCOC(=O)/C(=C/c1ccccc1Cl)CS(=O)c1ccccc1] |
| INF56 | 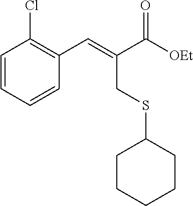 [SMILES: CCOC(=O)/C(=C/c1ccccc1Cl)CSC1CCCCC1] |
| INF57 | 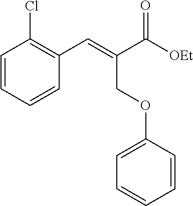 [SMILES: CCOC(=O)/C(=C/c1ccccc1Cl)COc1ccccc1] |
| INF43 | 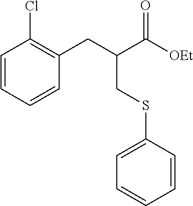 [SMILES: CCOC(=O)C(CSc1ccccc1)Cc1ccccc1Cl] |
| INF48 | 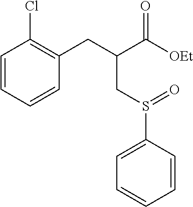 [SMILES: CCOC(=O)C(Cc1ccccc1Cl)CS(=O)c1ccccc1] |
| INF49 | 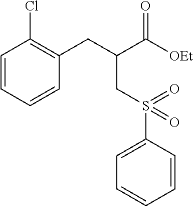 [SMILES: CCOC(=O)C(Cc1ccccc1Cl)CS(=O)(=O)c1ccccc1] |
| INF55 | 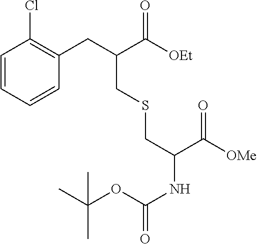 [SMILES: CCOC(=O)C(CSCC(NC(=O)OC(C)(C)C)C(=O)OC)Cc1ccccc1Cl] |
| INF110 | 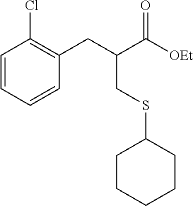 [SMILES: CCOC(=O)C(CSC1CCCCC1)Cc1ccccc1Cl] |
| INF85 | 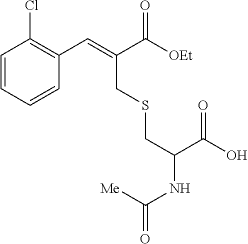 [SMILES: CCOC(=O)/C(=C/c1ccccc1Cl)CSCC(NC(C)=O)C(=O)O] |
| INF82 | 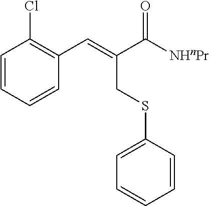 |
| INF80 | 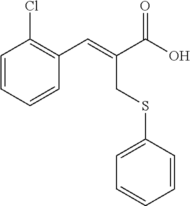 [SMILES: O=C(O)/C(=C/c1ccccc1Cl)CSc1ccccc1] |
| INF86 | 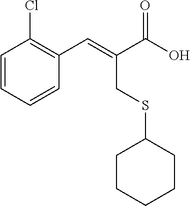 [SMILES: O=C(O)/C(=C/c1ccccc1Cl)CSC1CCCCC1] |
| INF111 | 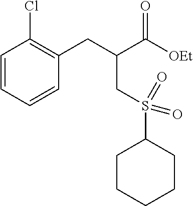 [SMILES: CCOC(=O)C(Cc1ccccc1Cl)CS(=O)(=O)C1CCCCC1] |
| INF176 | 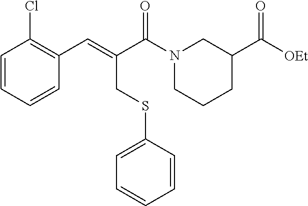 [SMILES: CCOC(=O)C1CCCN(C(=O)/C(=C/c2ccccc2Cl)CSc2ccccc2)C1] |
| INF202 | 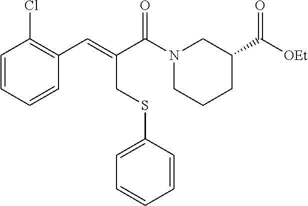 [SMILES: CCOC(=O)[C@@H]1CCCN(C(=O)/C(=C/c2ccccc2Cl)CSc2ccccc2)C1] |
| INF203 | 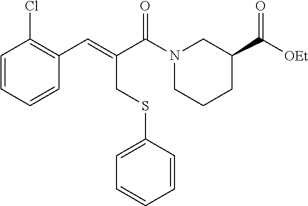 [SMILES: CCOC(=O)[C@H]1CCCN(C(=O)/C(=C/c2ccccc2Cl)CSc2ccccc2)C1] |
| INF177 | 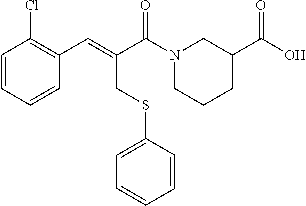 [SMILES: O=C(O)C1CCCN(C(=O)/C(=C/c2ccccc2Cl)CSc2ccccc2)C1] |
| INF180 | 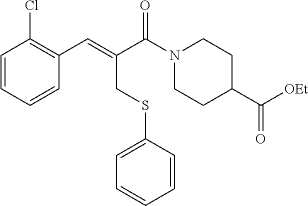 [SMILES: CCOC(=O)C1CCN(C(=O)/C(=C/c2ccccc2Cl)CSc2ccccc2)CC1] |
| INF184 | 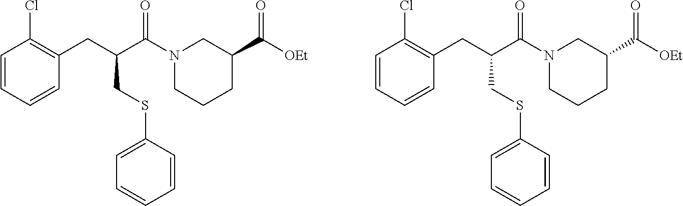 [SMILES: CCOC(=O)[C@H]1CCCN(C(=O)[C@@H](CSc2ccccc2)Cc2ccccc2Cl)C1 ; CCOC(=O)[C@@H]1CCCN(C(=O)[C@H](CSc2ccccc2)Cc2ccccc2Cl)C1] |
| INF185 | 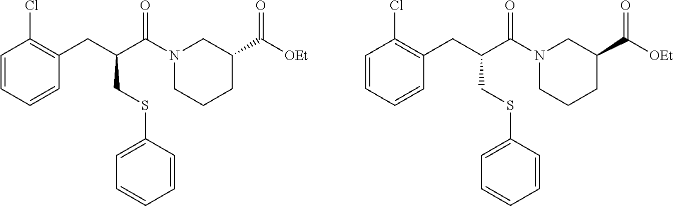 [SMILES: CCOC(=O)[C@@H]1CCCN(C(=O)[C@@H](CSc2ccccc2)Cc2ccccc2Cl)C1 ; CCOC(=O)[C@H]1CCCN(C(=O)[C@H](CSc2ccccc2)Cc2ccccc2Cl)C1] |
| INF186 | 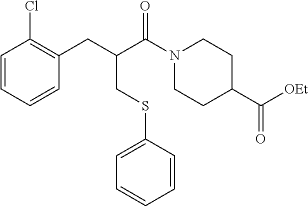 [SMILES: CCOC(=O)C1CCN(C(=O)C(CSc2ccccc2)Cc2ccccc2Cl)CC1] |
| INF187 | 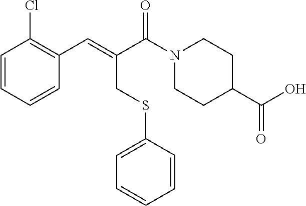 [SMILES: O=C(O)C1CCN(C(=O)/C(=C/c2ccccc2Cl)CSc2ccccc2)CC1] |
| INF188 | 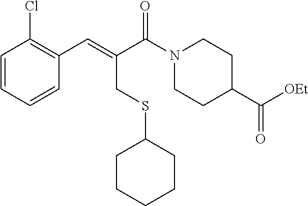 [SMILES: CCOC(=O)C1CCN(C(=O)/C(=C/c2ccccc2Cl)CSC2CCCCC2)CC1] |
| INF192 | 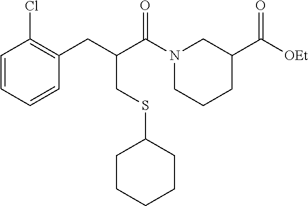 [SMILES: CCOC(=O)C1CCCN(C(=O)C(CSC2CCCCC2)Cc2ccccc2Cl)C1] |
| INF193 | 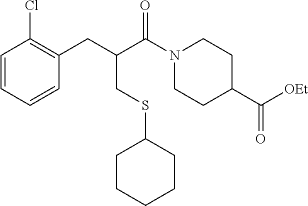 [SMILES: CCOC(=O)C1CCN(C(=O)C(CSC2CCCCC2)Cc2ccccc2Cl)CC1] |
| INF194 | 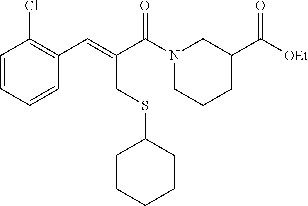 [SMILES: CCOC(=O)C1CCCN(C(=O)/C(=C/c2ccccc2Cl)CSC2CCCCC2)C1] |
| INF61 | 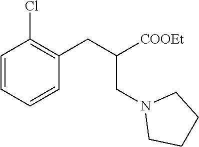 [SMILES: CCOC(=O)C(Cc1ccccc1Cl)CN1CCCC1] |
| INF37 syn | 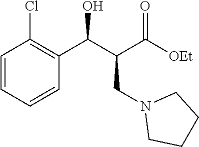 [SMILES: CCOC(=O)[C@@H](CN1CCCC1)[C@H](O)c1ccccc1Cl] |
| | 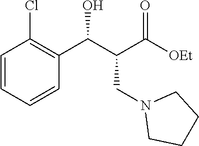 [SMILES: CCOC(=O)[C@H](CN1CCCC1)[C@@H](O)c1ccccc1Cl] |
| INF37 anti | 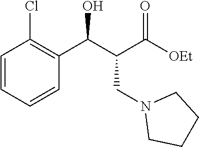 [SMILES: CCOC(=O)[C@H](CN1CCCC1)[C@H](O)c1ccccc1Cl] |
| | 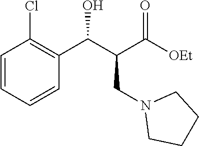 [SMILES: CCOC(=O)[C@@H](CN1CCCC1)[C@@H](O)c1ccccc1Cl] |
| INF219 | 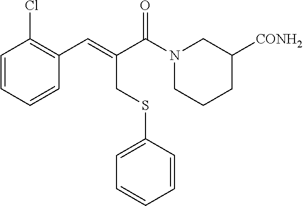 [SMILES: NC(=O)C1CCCN(C(=O)/C(=C/c2ccccc2Cl)CSc2ccccc2)C1] |
| INF220 | 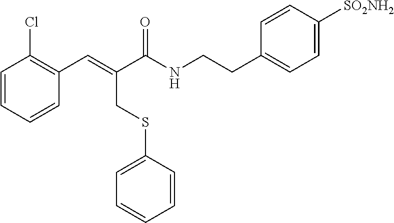 [SMILES: NS(=O)(=O)c1ccc(CCNC(=O)/C(=C/c2ccccc2Cl)CSc2ccccc2)cc1] |
| INF51 | 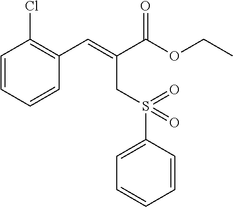 [SMILES: CCOC(=O)/C(=C/c1ccccc1Cl)CS(=O)(=O)c1ccccc1] |
| INF230 | 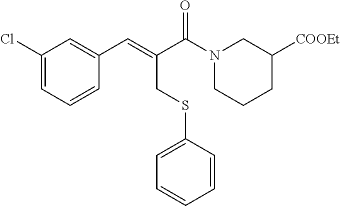 [SMILES: CCOC(=O)C1CCCN(C(=O)/C(=C/c2cccc(Cl)c2)CSc2ccccc2)C1] |
| INF231 | 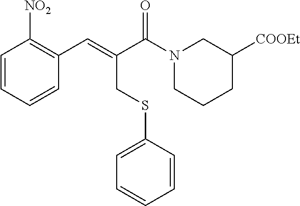 [SMILES: CCOC(=O)C1CCCN(C(=O)/C(=C/c2ccccc2[N+](=O)[O-])CSc2ccccc2)C1] |

6. The method according to claim 1, wherein the compound is selected from:

| Compound | Structure |
|---|---|
| Br1 | 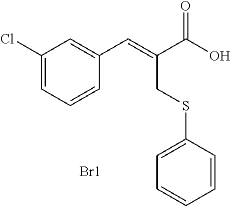 [SMILES: O=C(O)/C(=C/c1cccc(Cl)c1)CSc1ccccc1 ; "Br1"] |
| Br2S | 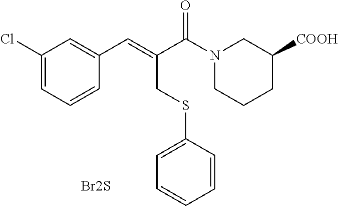 [SMILES: O=C(O)[C@H]1CCCN(C(=O)/C(=C/c2cccc(Cl)c2)CSc2ccccc2)C1 ; "Br2S"] |
| Br2R | 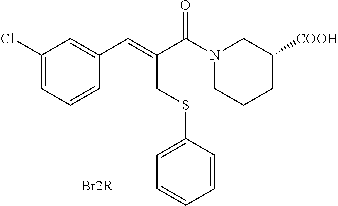 [SMILES: O=C(O)[C@@H]1CCCN(C(=O)/C(=C/c2cccc(Cl)c2)CSc2ccccc2)C1 ; "Br2R"] |
| Br4R | 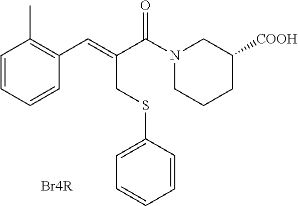 [SMILES: Cc1ccccc1/C=C(\CSc1ccccc1)C(=O)N1CCC[C@@H](C(=O)O)C1 ; "Br4R"] |
| Br7 | 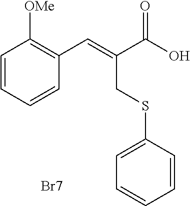 [SMILES: "Br7" ; COc1ccccc1/C=C(\CSc1ccccc1)C(=O)O] |
| Br8R | 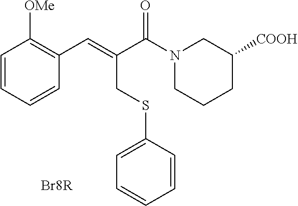 [SMILES: "Br8R" ; COc1ccccc1/C=C(\CSc1ccccc1)C(=O)N1CCC[C@@H](C(=O)O)C1] |
| Br9R | 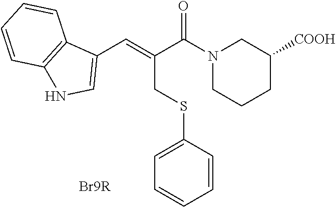 [SMILES: O=C(O)[C@@H]1CCCN(C(=O)/C(=C/c2c[nH]c3ccccc23)CSc2ccccc2)C1 ; "Br9R"] |
| Br10R | 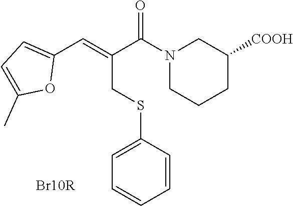 [SMILES: "Br10R" ; Cc1ccc(/C=C(\CSc2ccccc2)C(=O)N2CCC[C@@H](C(=O)O)C2)o1] |
| Br10S | 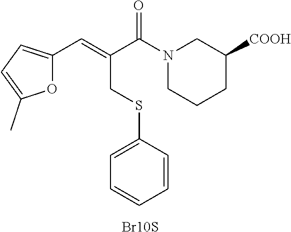 [SMILES: Cc1ccc(/C=C(\CSc2ccccc2)C(=O)N2CCC[C@H](C(=O)O)C2)o1 ; "Br10S"] |
| Br11R | 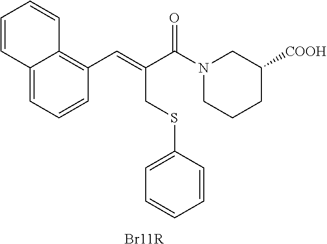 [SMILES: O=C(O)[C@@H]1CCCN(C(=O)/C(=C/c2cccc3ccccc23)CSc2ccccc2)C1 ; "Br11R"] |
| Br12 | 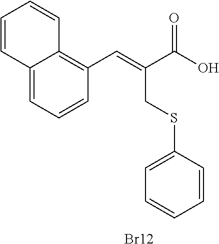 [SMILES: O=C(O)/C(=C/c1cccc2ccccc12)CSc1ccccc1 ; "Br12"] |
| Br13 | 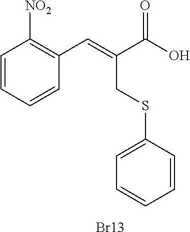 [SMILES: "Br13" ; O=C(O)/C(=C/c1ccccc1[N+](=O)[O-])CSc1ccccc1] |
| Br14R | 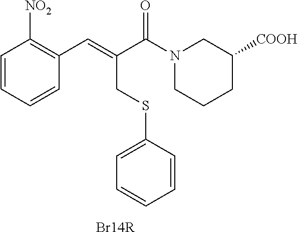 [SMILES: "Br14R" ; O=C(O)[C@@H]1CCCN(C(=O)/C(=C/c2ccccc2[N+](=O)[O-])CSc2ccccc2)C1] |
| Br14S | 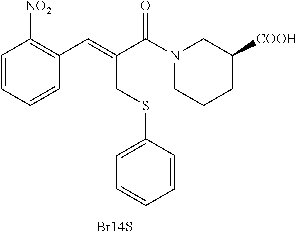 [SMILES: "Br14S" ; O=C(O)[C@H]1CCCN(C(=O)/C(=C/c2ccccc2[N+](=O)[O-])CSc2ccccc2)C1] |
| Br15 | 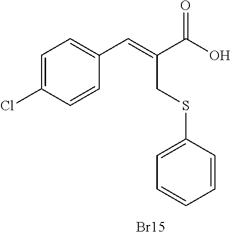 [SMILES: O=C(O)/C(=C/c1ccc(Cl)cc1)CSc1ccccc1 ; "Br15"] |
| Br16R | 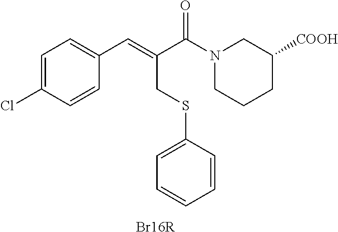 [SMILES: "Br16R" ; O=C(O)[C@@H]1CCCN(C(=O)/C(=C/c2ccc(Cl)cc2)CSc2ccccc2)C1] |
| Br17 | 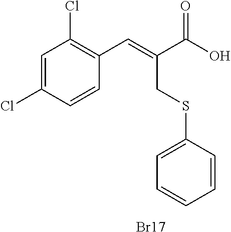 [SMILES: "Br17" ; O=C(O)/C(=C/c1ccc(Cl)cc1Cl)CSc1ccccc1] |
| Br18R | 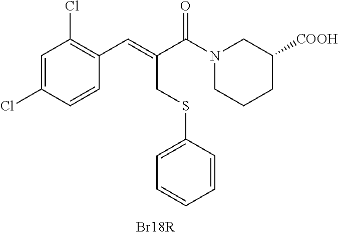 [SMILES: "Br18R" ; O=C(O)[C@@H]1CCCN(C(=O)/C(=C/c2ccc(Cl)cc2Cl)CSc2ccccc2)C1] |
| Br4S | 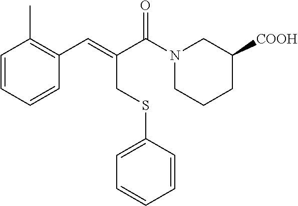 [SMILES: Cc1ccccc1/C=C(\CSc1ccccc1)C(=O)N1CCC[C@H](C(=O)O)C1] |
| Br8S | 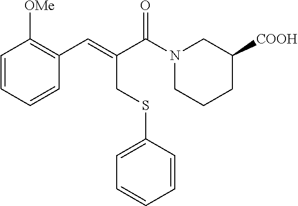 [SMILES: COc1ccccc1/C=C(\CSc1ccccc1)C(=O)N1CCC[C@H](C(=O)O)C1] |
| Br11S | 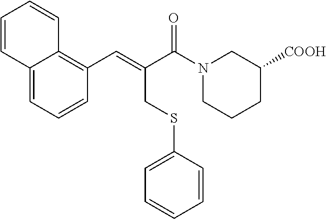 [SMILES: O=C(O)[C@@H]1CCCN(C(=O)/C(=C/c2cccc3ccccc23)CSc2ccccc2)C1] |

7. The method according to claim 1, wherein the compound is, an NLRP3 inflammasome-inhibiting medicament.

8. The method according to claim 1, wherein said diseases and/or disorders are inflammatory, autoimmune, neurodegenerative, cardiovascular, metabolic and neoplastic diseases and/or disorders.

9. The method according to claim 8, wherein the diseases and/or disorders are selected from:
cryopyrin-associated periodic syndromes (CAPS) which comprise familial cold autoinflammatory syndrome (FCAS), Muckle-Wells syndrome (MWS) and chronic infantile neurological cutaneous and articular syndrome (CINCA), also known as neonatal-onset multisystem inflammatory disease (NOMID);
asthma, chronic or acute inflammatory arthritis, osteoarthritis, rheumatoid arthritis, acute or chronic joint disease, psoriasis, sterile corneal inflammation, systemic sclerosis, ankylosing spondylitis, sepsis, chronic inflammatory bowel diseases, irritable bowel syndrome, inflammation induced by viral infections (such as those caused by the SARS-CoV-2 (COVID-19) virus);
Alzheimer's disease, multiple sclerosis, Parkinson's disease (PD), amyotrophic lateral sclerosis (ALS) and correlated symptoms (such as gastrointestinal disorders);
cardiovascular diseases (such as hypertension, myocardial infarction, diabetic cardiomyopathy, atherosclerosis, pericarditis and ischaemia);
non-alcoholic steatohepatitis (NASH), liver disease and correlated disorders comprising hepatic fibrosis;
obesity, type I diabetes, type II diabetes, kidney disease and correlated disorders comprising gastrointestinal disorders;
tumours comprising stomach cancer, head/neck cancer, lung cancer, melanoma and myelodysplastic syndromes.

10. Compounds of general formula (I): 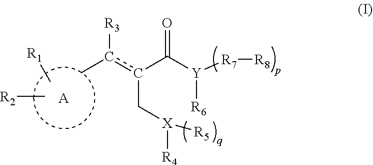
wherein
A is a C₃-C₁₀-cycloalkyl, preferably monocyclic or bicyclic C₅-C₁₀-cycloalkyl; 5- to 10-membered, saturated or partly saturated, monocyclic or bicyclic heterocycle; monocyclic or bicyclic C₆-C₁₀-aryl; 5- to 10-membered monocyclic or bicyclic heteroaryl; A is preferably a 5 or 6-membered, saturated or partly saturated monocyclic heterocycle, or a 9 or 10-membered, saturated or partly saturated bicyclic heterocycle; or a monocyclic C₅-C₆-aryl, or a bicyclic C₉-C₁₀-aryl; or a 5 or 6-membered monocyclic heteroaryl or a 9 or 10-membered bicyclic heteroaryl; wherein the heteroatom is preferably N or O; more preferably A is phenyl, naphthyl, furanyl or indolyl, and most preferably A is phenyl;
R₁ and R₂, which are the same or different, can occupy any position on A, and can be hydrogen; halogen such as F, Cl, Br or I; linear or branched, substituted or unsubstituted, saturated or unsaturated C₁-C₄-alkyl; linear or branched, substituted or unsubstituted, saturated or unsaturated C₁-C₄-alkoxy; a nitro group; nitrile; a substituted or unsubstituted amido group; a substituted or unsubstituted amino group; a substituted or unsubstituted ester group; a trifluoromethyl group; R₁ and R₂ are preferably hydrogen, halogen such as F, Cl, Br or I, linear or branched C₁-C₄-alkyl, linear or branched C₁-C₄-alkoxy, a nitro group; R₁ and R₂ are more preferably hydrogen, chloro, bromo, methyl, methoxy or a nitro group; most preferably R₁ is hydrogen and R₂ is chloro;
wherein at least one of R₁ and R₂ is other than hydrogen when A is phenyl,
wherein at least one of R₁ and R₂ is other than hydrogen when X is SO₂,
wherein at least one of R₁ and R₂ is preferably other than H and is in the 2 position when A is phenyl, and R₆ and R₇ do not form a ring, and the other R₁ or R₂ occupy any other position on A,
R₁ or R₂ is preferably a halogen such as F, Cl, Br or I, is in the 2 position when A is phenyl, and is more preferably Cl; [symbol]
is a single bond or a double bond;
R₃ is selected from —H, —OH, —OR₉ and —O(CO)R₉, wherein R₉ is hydrogen or a linear or branched, substituted or unsubstituted, saturated or unsaturated C₁-C₄-alkyl; R₃ is preferably hydrogen or —OH; R₃ is more preferably hydrogen;
in the 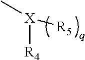
group, X is selected from N, O, S, S(O) and SO₂, or can be O, S, S(O) or SO₂ when Y is O;
R₄ can be a linear or branched, substituted or unsubstituted, saturated or unsaturated C₁₋₄ alkyl group; monocyclic or bicyclic C₃-C₁₀-cycloalkyl; substituted or unsubstituted, preferably a C₃-C₆-cycloalkyl; monocyclic or bicyclic C₆-C₁₄-aryl, substituted or unsubstituted, preferably a C₆-C₁₀-aryl, more preferably a C₅-C₆-aryl; 5- to 10-membered heterocycle, saturated or partly saturated, monocyclic or bicyclic, substituted or unsubstituted, preferably a C₅-C₆-heterocycle; monocyclic or polycyclic 5- to 14-membered heteroaryl, preferably monocyclic or bicyclic, substituted or unsubstituted, preferably a C₅-C₆-heteroaryl; R₄ is preferably monocyclic or bicyclic C₆-C₁₀-aryl, substituted or unsubstituted, or C₃-C₆-cycloalkyl, substituted or unsubstituted; more preferably, R₄ is cyclohexyl or phenyl;
q is 0 (zero) or 1; when q is equal to 1, X is N and R₅ is hydrogen; a linear or branched, substituted or unsubstituted, saturated or unsaturated C₁-C₄-alkyl group; monocyclic or bicyclic C₃-C₁₀-cycloalkyl;
alternatively, the 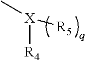
group is an amino-acid residue wherein:
X is an N, S or O atom, or an S or O atom when Y is O, of the side chain of an amino acid, preferably natural, selected from serine; tyrosine; threonine; lysine; cysteine; q is zero and R₄ is the remainder of the amino acid optionally protected on the terminal NH₂ and/or COOH groups; the terminal NH₂ group is preferably acetylated; the amino-acid residue is preferably N-acetylcysteine or N-Boc cysteine methyl ester; or
X is the N atom of the terminal amino group bonded to the stereogenic carbon atom in alpha of a preferably natural, optionally protected amino acid, selected from alanine, arginine, asparagine, aspartic acid, cysteine, glycine, glutamic acid, glutamine, histidine, isoleucine, leucine, lysine, methionine, phenylalanine, proline, serine, threonine, tryptophan, tyrosine and valine; q is equal to 1, R₅ is hydrogen; and R₄ represents the remainder of the amino-acid structure, optionally protected, for example acetylated on the N atom of the side chain or esterified with a linear or branched C₁-C₄-alkyl group, preferably methyl, on the terminal carboxyl group;
alternatively when, in the 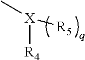
group, X is N, Y is other than O, R₄ and R₅ are joined to form a monocyclic or bicyclic, saturated, partly saturated or unsaturated C₃-C₁₀-heterocyclic ring with the N atom; R₄ and R₅ preferably form a monocyclic C₃-C₆-heterocyclic ring with the N atom; more preferably, R₄ and R₅ form a piperidine or pyrrolidine ring with the N atom; most preferably, R₄ and R₅ form a pyrrolidine ring with the N atom;
Y is selected from O, N and S; is preferably O or N; and is more preferably N;
when Y is an oxygen or sulfur atom, in the —(R₇-R₈)p group p is equal to zero and R₆ is selected from hydrogen, a linear or branched, substituted or unsubstituted, saturated or unsaturated C₁-C₈-alkyl group; a monocyclic or bicyclic C₃-C₁₀-cycloalkyl; a substituted or unsubstituted arylalkyl; a 6- to 14-membered monocyclic or bicyclic heteroaryl; R₆ is preferably hydrogen or a linear or branched, substituted or unsubstituted, saturated or unsaturated C₁-C₄-alkyl group; R₆ is more preferably a linear or branched, saturated, unsubstituted C₁-C₄-alkyl group; R₆ is most preferably ethyl;
when Y is a nitrogen atom, p is equal to 1, R₆ and R₇, which are the same or different, are selected from hydrogen, a linear or branched, saturated or unsaturated, substituted or unsubstituted C₁-C₄-alkyl group; a substituted or unsubstituted aryl, arylalkyl or heteroaryl group; are preferably a substituted phenylalkyl group; more preferably —(CH₂)₂-phenyl-SO₂NH₂; most preferably R₆ is hydrogen and R₇ is —(CH₂)₂-phenyl-SO₂NH₂;
R₈ is selected from H, COOH, COOR₉, C(O)R₉, CN, CONH(R₉), S(O)NHR₉ and S(O)₂NHR₉, wherein R₉ is as defined above;
alternatively, R₆ and R₇ are joined to form a 3- to 8-membered heterocyclic ring;
R₈ is as defined above;
and their enantiomers, diastereomers, rotamers or mixtures thereof;
and the pharmaceutically acceptable salts or solvates thereof;
wherein
in compounds of formula (I), when R₆ and R₇ do not form a ring:
when A is phenyl, R₁ and R₂ are as defined above, [symbol] is a double bond, R₃ is H or OH, Y is O, X is S, q and p are zero, R₄ is methyl, R₆ is hydrogen, a linear or branched, substituted or unsubstituted, saturated or unsaturated C₃-C₈-alkyl group; a monocyclic or bicyclic C₃-C₁₀-cycloalkyl; a substituted or unsubstituted arylalkyl; a 6- to 14-membered monocyclic or bicyclic heteroaryl; R₆ is preferably hydrogen or a linear or branched, substituted or unsubstituted, saturated or unsaturated C₃-C₆-alkyl group; and/or
when A is phenyl, naphthyl or thiophene, R₁ and R₂ are as defined above, R₃ is H, Y is O, X is SO₂, q and p are zero, R₄ is phenyl, ethyl or methyl, 4-chlorophenyl, 4-toluene, R₆ is methyl or ethyl, [symbol] is a single bond; and/or
when A is phenyl or naphthyl, R₁ and R₂ are as defined above, [symbol] is a double bond, R₃ is H, Y is O, X is O, q and p are zero, R₄ is methyl, R₆ is hydrogen, methyl, a linear or branched, substituted or unsubstituted, saturated or unsaturated C₃-C₈-alkyl group; a monocyclic or bicyclic C₃-C₁₀-cycloalkyl; a substituted or unsubstituted arylalkyl; a 6- to 14-membered monocyclic or bicyclic heteroaryl; R₆ is preferably hydrogen or a linear or branched, substituted or unsubstituted, saturated or unsaturated C₃-C₆-alkyl group; and/or
when A is phenyl, R₁ and R₂ are as defined above, [symbol] is a double bond, R₃ is H, Y is O, X is O, q and p are zero, R₄ is phenyl, R₆ is methyl, ethyl, a linear or branched, substituted or unsubstituted, saturated or unsaturated C₃-C₈-alkyl group; a monocyclic or bicyclic C₃-C₁₀-cycloalkyl; a substituted or unsubstituted arylalkyl; a 6- to 14-membered monocyclic or bicyclic heteroaryl; R₆ is preferably hydrogen or a linear or branched, substituted or unsubstituted, saturated or unsaturated C₃-C₆-alkyl group; and/or
when A is phenyl, R₁ and R₂ are as defined above, Y is O and X is N, p is zero, R₆ is methyl, q is one, [symbol] is preferably a double bond, and is more preferably a single bond or a double bond; R₄ and R₅ are joined to form a monocyclic or bicyclic, saturated, partly saturated or unsaturated C₃-C₁₀-heterocyclic ring with the N atom; R₄ and R₅ preferably form a monocyclic C₃-C₆-heterocyclic ring with the N atom; more preferably, R₄ and R₅ form a piperidine or pyrrolidine ring with the N atom; most preferably R₄ and R₅ form a pyrrolidine ring with the N atom.

11. Compounds according to claim 10 having general formula (Ia): 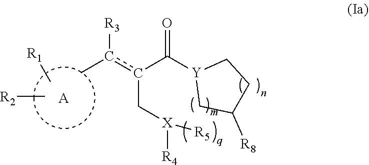
wherein
A, R₁, R₂, R₃, R₄, R₅, R₈, q, X and Y are as defined in claim 10,
n and m, which are the same or different, are 0 (zero) or an integer between 1 and 3; preferably m is 2 and n is 1; more preferably n is 2 and m is 1 or 2; most preferably, n is 2 and m is 1;
preferably, when Y is N, R₆ and R₇ are joined to form a 3- to 6-membered monocyclic substituted heterocyclic ring with the N atom; more preferably, R₆ and R₇ form a substituted piperidine or pyrrolidine ring with the N atom; most preferably, R₆ and R₇ form, with the N atom, a piperidine ring substituted in the 3 or 4 position;
and their enantiomers, diastereomers, rotamers or mixtures thereof;
and the pharmaceutically acceptable salts or solvates thereof.

12. Compounds according to claim 10 having general formula (Ib): 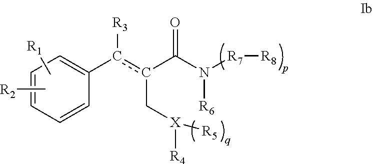
wherein
R₁, R₂, R₃, R₄, R₅, R₆, R₇, R₈, q, p, X and Y are as defined in claim 10,
and their enantiomers, diastereomers, rotamers or mixtures thereof;
and the pharmaceutically acceptable salts or solvates thereof.

13. Compounds according to claim 10 having general formula (Ic): 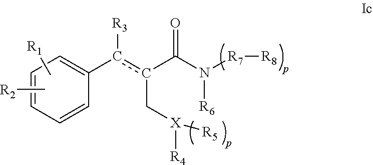
wherein
R₁, R₂, R₃, R₄, R₅, R₆, R₇, R₈, q, p and X are as defined in claim 10,
and their enantiomers, diastereomers, rotamers or mixtures thereof;
and the pharmaceutically acceptable salts or solvates thereof.

14. Compounds according to claim 10 selected from:

| Compound | Structure |
|---|---|
| INF38s | 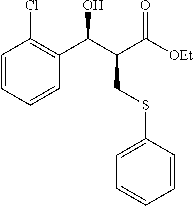 [SMILES: CCOC(=O)[C@@H](CSc1ccccc1)[C@H](O)c1ccccc1Cl] |
| | 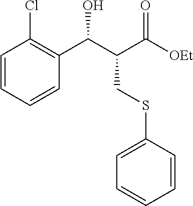 [SMILES: CCOC(=O)[C@H](CSc1ccccc1)[C@@H](O)c1ccccc1Cl] |
| INF38a | 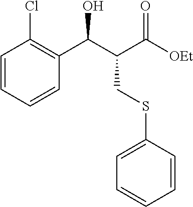 [SMILES: CCOC(=O)[C@H](CSc1ccccc1)[C@H](O)c1ccccc1Cl] |
| | 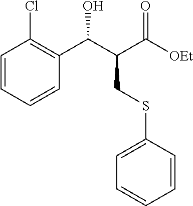 [SMILES: CCOC(=O)[C@@H](CSc1ccccc1)[C@@H](O)c1ccccc1Cl] |
| INF44 | 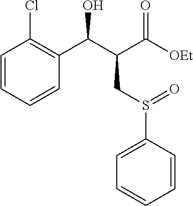 [SMILES: CCOC(=O)[C@@H](CS(=O)c1ccccc1)[C@H](O)c1ccccc1Cl] |
| | 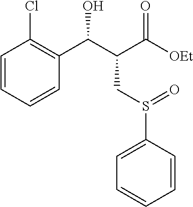 [SMILES: CCOC(=O)[C@H](CS(=O)c1ccccc1)[C@@H](O)c1ccccc1Cl] |
| INF45 | 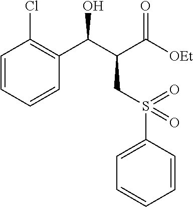 [SMILES: CCOC(=O)[C@@H](CS(=O)(=O)c1ccccc1)[C@H](O)c1ccccc1Cl] |
| | 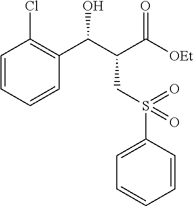 [SMILES: CCOC(=O)[C@H](CS(=O)(=O)c1ccccc1)[C@@H](O)c1ccccc1Cl] |
| INF42 | 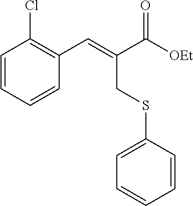 [SMILES: CCOC(=O)/C(=C/c1ccccc1Cl)CSc1ccccc1] |
| INF50 | 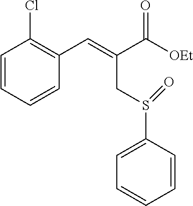 [SMILES: CCOC(=O)/C(=C/c1ccccc1Cl)CS(=O)c1ccccc1] |
| INF56 | 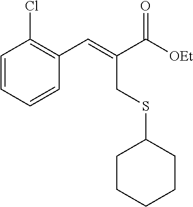 [SMILES: CCOC(=O)/C(=C/c1ccccc1Cl)CSC1CCCCC1] |
| INF57 | 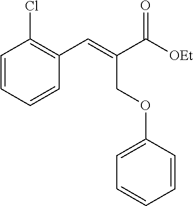 [SMILES: CCOC(=O)/C(=C/c1ccccc1Cl)COc1ccccc1] |
| INF43 | 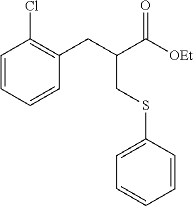 [SMILES: CCOC(=O)C(CSc1ccccc1)Cc1ccccc1Cl] |
| INF48 | 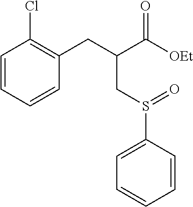 [SMILES: CCOC(=O)C(Cc1ccccc1Cl)CS(=O)c1ccccc1] |
| INF49 | 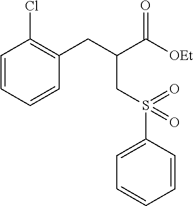 [SMILES: CCOC(=O)C(Cc1ccccc1Cl)CS(=O)(=O)c1ccccc1] |
| INF55 | 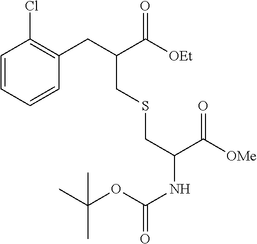 [SMILES: CCOC(=O)C(CSCC(NC(=O)OC(C)(C)C)C(=O)OC)Cc1ccccc1Cl] |
| INF110 | 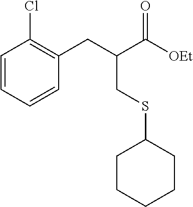 [SMILES: CCOC(=O)C(CSC1CCCCC1)Cc1ccccc1Cl] |
| INF85 | 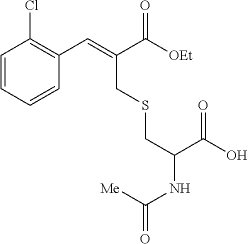 [SMILES: CCOC(=O)/C(=C/c1ccccc1Cl)CSCC(NC(C)=O)C(=O)O] |
| INF82 | 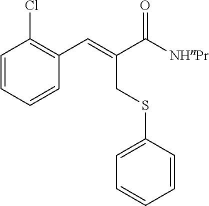 |
| INF80 | 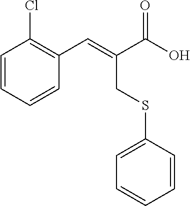 [SMILES: O=C(O)/C(=C/c1ccccc1Cl)CSc1ccccc1] |
| INF86 | 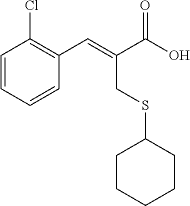 [SMILES: O=C(O)/C(=C/c1ccccc1Cl)CSC1CCCCC1] |
| INF111 | 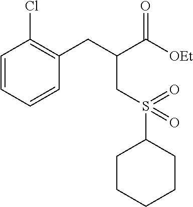 [SMILES: CCOC(=O)C(Cc1ccccc1Cl)CS(=O)(=O)C1CCCCC1] |
| INF176 | 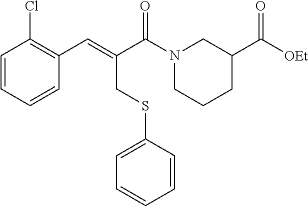 [SMILES: CCOC(=O)C1CCCN(C(=O)/C(=C/c2ccccc2Cl)CSc2ccccc2)C1] |
| INF202 | 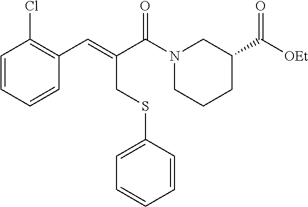 [SMILES: CCOC(=O)[C@@H]1CCCN(C(=O)/C(=C/c2ccccc2Cl)CSc2ccccc2)C1] |
| INF203 | 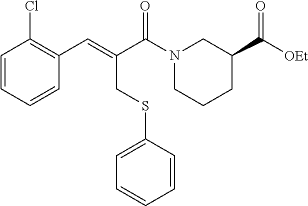 [SMILES: CCOC(=O)[C@H]1CCCN(C(=O)/C(=C/c2ccccc2Cl)CSc2ccccc2)C1] |
| INF177 | 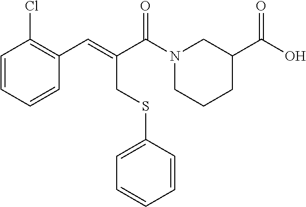 [SMILES: O=C(O)C1CCCN(C(=O)/C(=C/c2ccccc2Cl)CSc2ccccc2)C1] |
| INF180 | 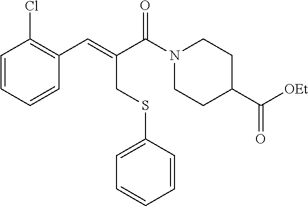 [SMILES: CCOC(=O)C1CCN(C(=O)/C(=C/c2ccccc2Cl)CSc2ccccc2)CC1] |
| INF184 | 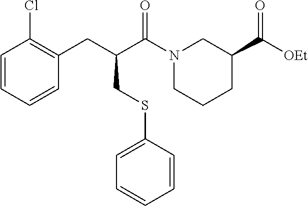 [SMILES: CCOC(=O)[C@H]1CCCN(C(=O)[C@@H](CSc2ccccc2)Cc2ccccc2Cl)C1] |
| | 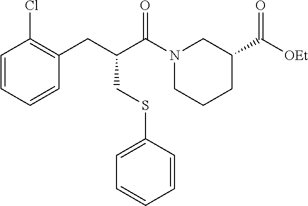 [SMILES: CCOC(=O)[C@@H]1CCCN(C(=O)[C@H](CSc2ccccc2)Cc2ccccc2Cl)C1] |
| INF185 | 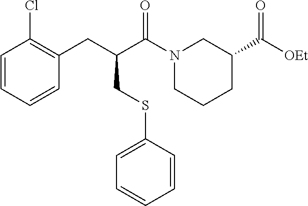 [SMILES: CCOC(=O)[C@@H]1CCCN(C(=O)[C@@H](CSc2ccccc2)Cc2ccccc2Cl)C1] |
| | 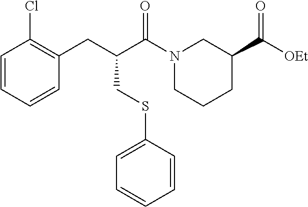 [SMILES: CCOC(=O)[C@H]1CCCN(C(=O)[C@H](CSc2ccccc2)Cc2ccccc2Cl)C1] |
| INF186 | 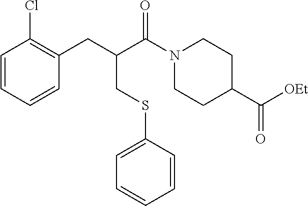 [SMILES: CCOC(=O)C1CCN(C(=O)C(CSc2ccccc2)Cc2ccccc2Cl)CC1] |
| INF187 | 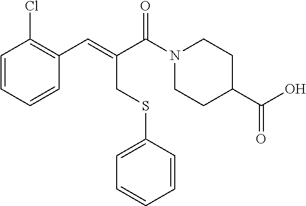 [SMILES: O=C(O)C1CCN(C(=O)/C(=C/c2ccccc2Cl)CSc2ccccc2)CC1] |
| INF188 | 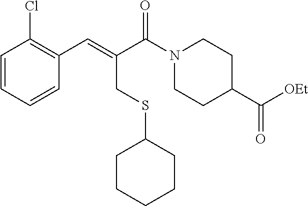 [SMILES: CCOC(=O)C1CCN(C(=O)/C(=C/c2ccccc2Cl)CSC2CCCCC2)CC1] |
| INF192 | 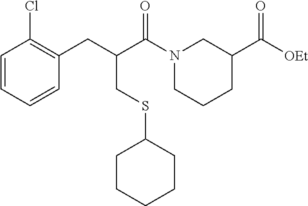 [SMILES: CCOC(=O)C1CCCN(C(=O)C(CSC2CCCCC2)Cc2ccccc2Cl)C1] |
| INF193 | 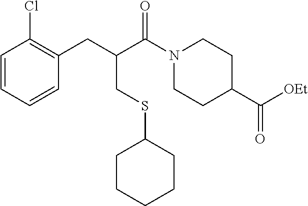 [SMILES: CCOC(=O)C1CCN(C(=O)C(CSC2CCCCC2)Cc2ccccc2Cl)CC1] |
| INF194 | 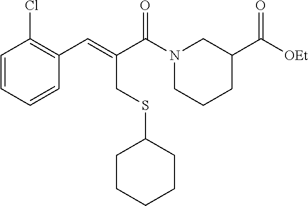 [SMILES: CCOC(=O)C1CCCN(C(=O)/C(=C/c2ccccc2Cl)CSC2CCCCC2)C1] |
| INF219 | 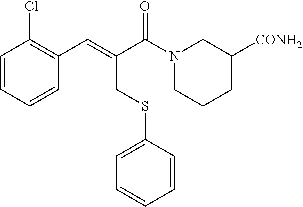 [SMILES: NC(=O)C1CCCN(C(=O)/C(=C/c2ccccc2Cl)CSc2ccccc2)C1] |
| INF220 | 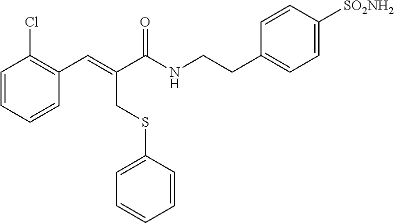 [SMILES: NS(=O)(=O)c1ccc(CCNC(=O)/C(=C/c2ccccc2Cl)CSc2ccccc2)cc1] |
| INF230 | 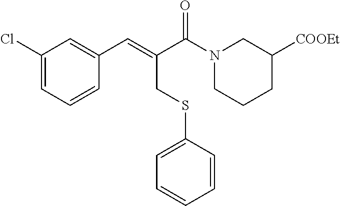 [SMILES: CCOC(=O)C1CCCN(C(=O)/C(=C/c2cccc(Cl)c2)CSc2ccccc2)C1] |
| INF231 | 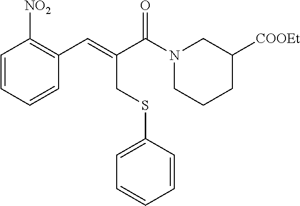 [SMILES: CCOC(=O)C1CCCN(C(=O)/C(=C/c2ccccc2[N+](=O)[O-])CSc2ccccc2)C1] |

15. Compounds according to claim 10, selected from:

| Compound | Structure |
|---|---|
| Br1 | 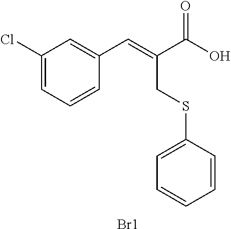 [SMILES: O=C(O)/C(=C/c1cccc(Cl)c1)CSc1ccccc1 ; "Br1"] |
| Br2S | 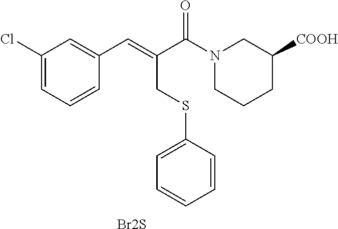 [SMILES: O=C(O)[C@H]1CCCN(C(=O)/C(=C/c2cccc(Cl)c2)CSc2ccccc2)C1 ; "Br2S"] |
| Br2R | 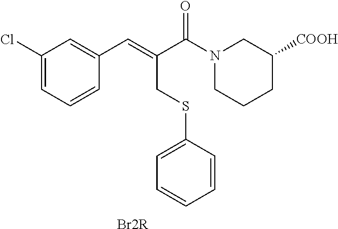 [SMILES: O=C(O)[C@@H]1CCCN(C(=O)/C(=C/c2cccc(Cl)c2)CSc2ccccc2)C1 ; "Br2R"] |
| Br4R | 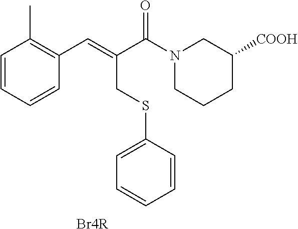 [SMILES: Cc1ccccc1/C=C(\CSc1ccccc1)C(=O)N1CCC[C@@H](C(=O)O)C1 ; "Br4R"] |
| Br7 | 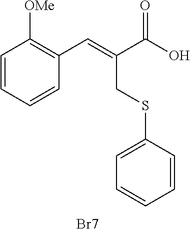 [SMILES: COc1ccccc1/C=C(\CSc1ccccc1)C(=O)O ; "Br7"] |
| Br8R | 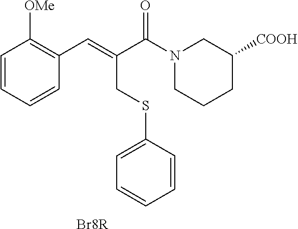 [SMILES: COc1ccccc1/C=C(\CSc1ccccc1)C(=O)N1CCC[C@@H](C(=O)O)C1 ; "Br8R"] |
| Br9R | 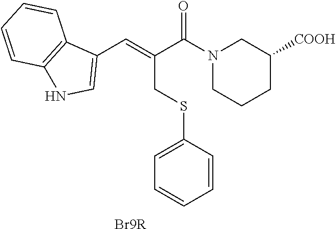 [SMILES: O=C(O)[C@@H]1CCCN(C(=O)/C(=C/c2c[nH]c3ccccc23)CSc2ccccc2)C1 ; "Br9R"] |
| Br10R | 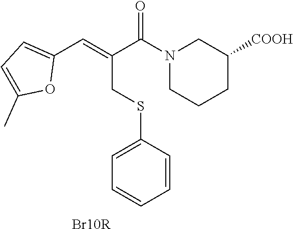 [SMILES: "Br10R" ; Cc1ccc(/C=C(\CSc2ccccc2)C(=O)N2CCC[C@@H](C(=O)O)C2)o1] |
| Br10S | 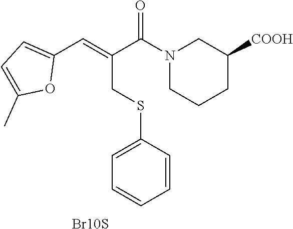 [SMILES: "Br10S" ; Cc1ccc(/C=C(\CSc2ccccc2)C(=O)N2CCC[C@H](C(=O)O)C2)o1] |
| Br11R | 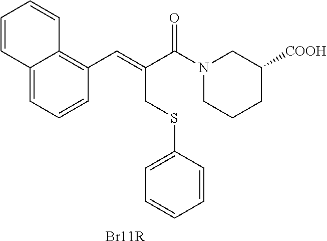 [SMILES: O=C(O)[C@@H]1CCCN(C(=O)/C(=C/c2cccc3ccccc23)CSc2ccccc2)C1 ; "Br11R"] |
| Br12 | 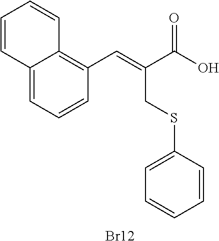 [SMILES: O=C(O)/C(=C/c1cccc2ccccc12)CSc1ccccc1 ; "Br12"] |
| Br13 | 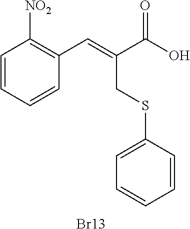 [SMILES: "Br13" ; O=C(O)/C(=C/c1ccccc1[N+](=O)[O-])CSc1ccccc1] |
| Br14R | 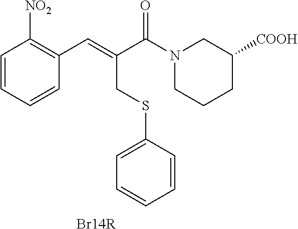 [SMILES: "Br14R" ; O=C(O)[C@@H]1CCCN(C(=O)/C(=C/c2ccccc2[N+](=O)[O-])CSc2ccccc2)C1] |
| Br14S | 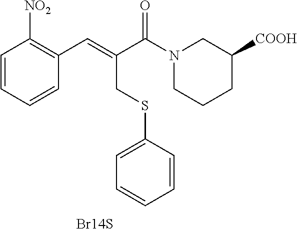 [SMILES: "Br14S" ; O=C(O)[C@H]1CCCN(C(=O)/C(=C/c2ccccc2[N+](=O)[O-])CSc2ccccc2)C1] |
| Br15 | 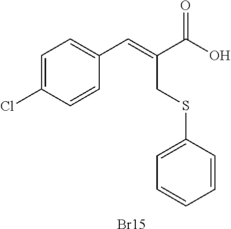 [SMILES: "Br15" ; O=C(O)/C(=C/c1ccc(Cl)cc1)CSc1ccccc1] |
| Br16R | 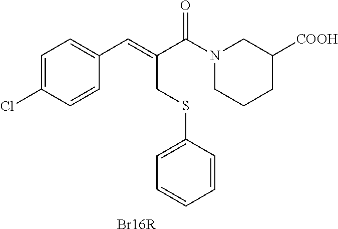 [SMILES: "Br16R" ; O=C(O)C1CCCN(C(=O)/C(=C/c2ccc(Cl)cc2)CSc2ccccc2)C1] |
| Br17 | 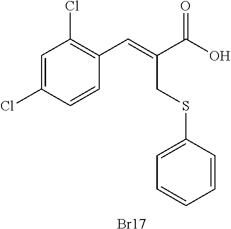 [SMILES: "Br17" ; O=C(O)/C(=C/c1ccc(Cl)cc1Cl)CSc1ccccc1] |
| Br18R | 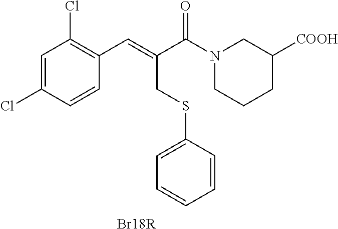 [SMILES: "Br18R" ; O=C(O)C1CCCN(C(=O)/C(=C/c2ccc(Cl)cc2Cl)CSc2ccccc2)C1] |
| Br4S | 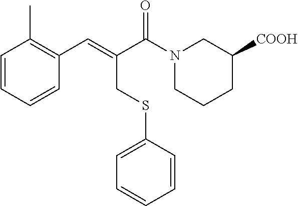 [SMILES: Cc1ccccc1/C=C(\CSc1ccccc1)C(=O)N1CCC[C@H](C(=O)O)C1] |
| Br8S | 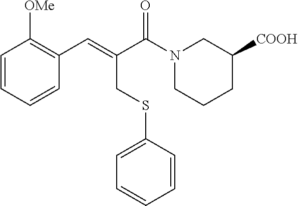 [SMILES: COc1ccccc1/C=C(\CSc1ccccc1)C(=O)N1CCC[C@H](C(=O)O)C1] |
| Br11S | 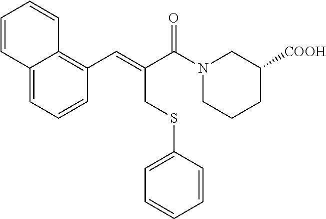 [SMILES: O=C(O)[C@@H]1CCCN(C(=O)/C(=C/c2cccc3ccccc23)CSc2ccccc2)C1] |

16. Medicament comprising the compounds
according to claim 10.

17. Pharmaceutical composition comprising at least one compound according to claim 10, and at least one pharmaceutically acceptable excipient.
